SEQUENCE LISTING (64 sequences)

SEQ ID NO: 1 - protein, 5 aa, synthetic construct
GYNMN

SEQ ID NO: 2 - protein, 17 aa, synthetic construct
NIDPYYGGTSYNQKFKG

SEQ ID NO: 3 - protein, 9 aa, synthetic construct
MYHGNAFDY

SEQ ID NO: 4 - protein, 17 aa, synthetic construct
KSSQSLLNSGNLKNYLT

SEQ ID NO: 5 - protein, 7 aa, synthetic construct
WASTRKS

SEQ ID NO: 6 - protein, 9 aa, synthetic construct
QNDYSYPLT

SEQ ID NO: 7 - protein, 118 aa, synthetic construct
EFQLQQSGPELEKPGASVRISCKTSGYSFTGYNMNWVKQSNGESLEWIGNIDPYYGGTSYNQKFKGKATLTVDKSSSTAYMQLKSLTSEDSAVYYCARMYHGNAFDYWGQGTTLTVSS

SEQ ID NO: 8 - protein, 113 aa, synthetic construct
DIVMTQSPSSLTVTAGEKVTMSCKSSQSLLNSGNLKNYLTWYQQKPGQPPKLLIYWASTRKSGVPDRFTGSGSGTDFTLTLSSVQAEDLAVYYCQNDYSYPLTFGAGTKLELK

SEQ ID NO: 9 - protein, 330 aa, synthetic construct [sequence omitted]

SEQ ID NO: 10 - protein, 107 aa, synthetic construct [sequence omitted]

SEQ ID NO: 11 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELVGGPSVFLLPPKPKDTLMISRTPEVTCVVVDVSHEDPEVKFNWYVDGVEVHNAKTKPPEEQYNSTLRVVSVLTVLHQDWLNGKEYKCKVSNKALPAPIEKTISKAKGQPREPQVYTLPPSRDELTKNQVSLTCLVKGFYPSDIAVEWESNGQPENNYKTTPLVLDSDGSFFLYSKLTVDKSRWQQGNVFSCSVMHEALHNHYTQKSLSLSPGK

SEQ ID NO: 12 - protein, 118 aa, synthetic construct
QVQLVQSGAEVKKPGASVKVSCKASGYTFTGYNMNWVRQAPGQGLEWMGNIDPYYGGTSYNQKFKGRVTMTIDKSTSTVYMELSSLRSEDTAVYYCARMYHGNAFDYWGQGTTVTVSS

SEQ ID NO: 13 - protein, 118 aa, synthetic construct
QVQLVQSGAEVKKPGASVKVSCKASGYSFTGYNMNWVRQAPGQGLEWMGNIDPYYGGTSYNQKFKGRVTLTIDKSTSTVYMELSSLRSEDTAVYYCARMYHGNAFDYWGQGTTVTVSS

SEQ ID NO: 14 - protein, 118 aa, synthetic construct
QVQLVQSGAEVKKPGASVKVSCKASGYSFTGYNMNWVKQAPGQGLEWIGNIDPYYGGTSYNQKFKGRVTLTIDKSTSTVYMELSSLRSEDTAVYYCARMYHGNAFDYWGQGTTVTVSS

SEQ ID NO: 15 - protein, 113 aa, synthetic construct
DIVMTQSPDSLAVSLGERATINCKSSQSLLNSGNLKNYLTWYQQKPGQPPKLLIYWASTRKSGVPDRFSGSGSGTDFTLTISSLQAEDVAVYYCQNDYSYPLTFGGGTKVEIK

SEQ ID NO: 16 - protein, 113 aa, synthetic construct
DIVMTQSPDSLAVSLGERATMNCKSSQSLLNSGNLKNYLTWYQQKPGQPPKLLIYWASTRKSGVPDRFTGSGSGTDFTLTISSLQAEDVAVYYCQNDYSYPLTFGGGTKVEIK

SEQ ID NO: 17 - protein, 117 aa, synthetic construct
QVQLVQSGAEVKKPGASVKVSCKASGYVFTDYYMNWVRQAPGQSLEWMGDINPNNAETLYNHKFKGRVTVTVDKSISTAYMELSRLRSDDTAVYYCVKWGDGPFAYWGQGTLVTVSS

SEQ ID NO: 18 - protein, 106 aa, synthetic construct
DIQMTQSPSSLSASVGDRVTITCKASQNVGAAVAWYQQKPGKAPKLLIYSVSDRYTGVPSRFSGSGSGTDFTLTISSLQPEDIATYYCQQYSNYPTFGQGTKLEIK

SEQ ID NO: 19 - protein, 447 aa, synthetic construct
QVQLVQSGAEVKKPGASVKVSCKASGYVFTDYYMNWVRQAPGQSLEWMGDINPNNAETLYNHKFKGRVTVTVDKSISTAYMELSRLRSDDTAVYYCVKWGDGPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGA

SEQ ID NO: 20 - protein, 447 aa, synthetic construct
QVQLVQSGAEVKKPGASVKVSCKASGYVFTDYYMNWVRQAPGQSLEWMGDINPNNAETLYNHKFKGRVTVTVDKSISTAYMELSRLRSDDTAVYYCVKWGDGPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 213 aa, synthetic construct
DIQMTQSPSSLSASVGDRVTITCKASQNVGAAVAWYQQKPGKAPKLLIYSVSDRYTGVPSRFSGSGSGTDFTLTISSLQPEDIATYYCQQYSNYPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 5 aa, synthetic construct
DYYMN

SEQ ID NO: 23 - protein, 17 aa, synthetic construct
DINPNNAETLYNHKFKG

SEQ ID NO: 24 - protein, 8 aa, synthetic construct
WGDGPFAY

SEQ ID NO: 25 - protein, 11 aa, synthetic construct
KASQNVGAAVA

SEQ ID NO: 26 - protein, 7 aa, synthetic construct
SVSDRYT

SEQ ID NO: 27 - protein, 8 aa, synthetic construct
QQYSNYPT

SEQ ID NO: 28 - protein, 119 aa, synthetic construct
QVQLVQSGAEVKKPGASVKLSCKASGYIFTTYWMHWVKQRPGQGLEWIGMIQPNSGGTKYNEKFKKKATLTVDKSISTAYMELSRLTSDDTAVYYCARGAGTVDYFDYWGQGSTLTVSS

SEQ ID NO: 29 - protein, 111 aa, synthetic construct
DIVLTQSPASLAVSVGQRATITCRASESVDIYGNSFMHWYQQKPGQPPKLLIYRASNLESGIPARFSGSGSRTDFTLTINPVEAQDTATYYCQQSTEDPYTFGGGTKLEIK

SEQ ID NO: 30 - protein, 449 aa, synthetic construct
QVQLVQSGAEVKKPGASVKLSCKASGYIFTTYWMHWVKQRPGQGLEWIGMIQPNSGGTKYNEKFKKKATLTVDKSISTAYMELSRLTSDDTAVYYCARGAGTVDYFDYWGQGSTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 218 aa, synthetic construct
DIVLTQSPASLAVSVGQRATITCRASESVDIYGNSFMHWYQQKPGQPPKLLIYRASNLESGIPARFSGSGSRTDFTLTINPVEAQDTATYYCQQSTEDPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 5 aa, synthetic construct
TYWMH

SEQ ID NO: 33 - protein, 17 aa, synthetic construct
MIQPNSGGTKYNEKFKK

SEQ ID NO: 34 - protein, 10 aa, synthetic construct
GAGTVDYFDY

SEQ ID NO: 35 - protein, 15 aa, synthetic construct
RASESVDIYGNSFMH

SEQ ID NO: 36 - protein, 7 aa, synthetic construct
RASNLES

SEQ ID NO: 37 - protein, 9 aa, synthetic construct
QQSTEDPYT

SEQ ID NO: 38 - protein, 261 aa, synthetic construct
MAVTACQGLGFVVSLIGIAGIIAATCMDQWSTQDLYNNPVTAVFNYQGLWRSCVRESSGFTECRGYFTLLGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV

SEQ ID NO: 39 - protein, 448 aa, synthetic construct
QVQLVQSGAEVKKPGASVKVSCKASGYTFTGYNMNWVRQAPGQGLEWMGNIDPYYGGTSYNQKFKGRVTMTIDKSTSTVYMELSSLRSEDTAVYYCARMYHGNAFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 220 aa, synthetic construct
DIVMTQSPDSLAVSLGERATINCKSSQSLLNSGNLKNYLTWYQQKPGQPPKLLIYWASTRKSGVPDRFSGSGSGTDFTLTISSLQAEDVAVYYCQNDYSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 441 aa, synthetic construct
QVQLQQSGPELVRPGASVKISCKASGYRFTRNYFHWVKQRPGQGLEWIGWIYPGGFDIEYSEKFKGKATLTTDTSSSTAYMLLTSLTSEDSAVYYCAINYGSTFGYWGQGTLVTVSVAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGK

SEQ ID NO: 42 - protein, 219 aa, synthetic construct
DVLMTQTPLSLPVSLGDQASISCRSSQNIVHSNGNTYLEWYLQRPGQSPKLLIYKVSNRFSGVPDRFSGSGSGTDFTLKINRVEAEDLGVYYCFQGSHVPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 43 - protein, 117 aa, synthetic construct
QVQLQQSGPELVRPGASVKISCKASGYRFTRNYFHWVKQRPGQGLEWIGWIYPGGFDIEYSEKFKGKATLTTDTSSSTAYMLLTSLTSEDSAVYYCAINYGSTFGYWGQGTLVTVSV

SEQ ID NO: 44 - protein, 112 aa, synthetic construct
DVLMTQTPLSLPVSLGDQASISCRSSQNIVHSNGNTYLEWYLQRPGQSPKLLIYKVSNRFSGVPDRFSGSGSGTDFTLKINRVEAEDLGVYYCFQGSHVPFTFGSGTKLEIK

SEQ ID NO: 45 - protein, 5 aa, synthetic construct
DSWIH

SEQ ID NO: 46 - protein, 17 aa, synthetic construct
WISPYGGSTYYADSVKG

SEQ ID NO: 47 - protein, 9 aa, synthetic construct
RHWPGGFDY

SEQ ID NO: 48 - protein, 11 aa, synthetic construct
RASQDVSTAVA

SEQ ID NO: 49 - protein, 7 aa, synthetic construct
SASFLYS

SEQ ID NO: 50 - protein, 9 aa, synthetic construct
QQYLYHPAT

SEQ ID NO: 51 - protein, 118 aa, synthetic construct [sequence omitted]

SEQ ID NO: 52 - protein, 107 aa, synthetic construct [sequence omitted]

SEQ ID NO: 53 - protein, 448 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 214 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 5 aa, synthetic construct
NSGMH

SEQ ID NO: 56 - protein, 17 aa, synthetic construct
VIWYDGSKRYYADSVKG

SEQ ID NO: 57 - protein, 4 aa, synthetic construct
NDDY

SEQ ID NO: 58 - protein, 11 aa, synthetic construct
RASQSVSSYLA

SEQ ID NO: 59 - protein, 7 aa, synthetic construct
DASNRAT

SEQ ID NO: 60 - protein, 9 aa, synthetic construct
QQSSNWPRT

SEQ ID NO: 61 - protein, 113 aa, synthetic construct [sequence omitted]

SEQ ID NO: 62 - protein, 107 aa, synthetic construct [sequence omitted]

SEQ ID NO: 63 - protein, 440 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 64 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0326834 A1
(43) Pub. Date: Oct. 23, 2025

(54) COMBINATION THERAPY OF CLAUDIN 18.2 ANTAGONIST AND PD-1/PD-L1 AXIS INHIBITOR

(71) Applicants: SUZHOU TRANSCENTA THERAPEUTICS CO., LTD., Suzhou, Jiangsu (CN); TRANSCENTA HOLDING LIMITED, Grand Cayman (KY); TRANSCENTA THERAPEUTICS, INC., Princeton, NJ (US)

(72) Inventors: Xueming QIAN, Suzhou, Jiangsu (CN); Xinlai YAO, Suzhou, Jiangsu (CN); Huanhuan GUO, Suzhou, Jiangsu (CN); Fei TENG, Suzhou, Jiangsu (CN); Wei YI, Suzhou, Jiangsu (CN)

(21) Appl. No.: 18/710,628

(22) PCT Filed: Nov. 15, 2022
(86) PCT No.: PCT/CN2022/131820

§ 371 Date: May 16, 2024

(30) Foreign Application Priority Data
Nov. 16, 2021 (WO) PCT/CN2021/130995
Oct. 13, 2022 (WO) PCT/CN2022/125150

(51) Int. Cl. C07K 16/28; A61K 39/00; A61K 40/31; A61K 40/32; A61K 40/42; A61K 45/06; G01N 33/68

(52) U.S. Cl. CPC C07K 16/28; A61K 40/31; A61K 40/32; A61K 40/421; A61K 45/06; C07K 16/2818; C07K 16/2827; G01N 33/68; A61K 2039/507; C07K 2317/31; C07K 2317/52; C07K 2317/565; C07K 2317/76; G01N 2333/705; G01N 2800/00

(57) ABSTRACT

Provided are combination therapies of a CLDN 18.2 antagonist and a PD-1/PD-L1 axis inhibitor for subjects having expression of CLDN 18.2 and having low or no expression of PD-L1 in a disease tissue (e.g., tumor tissue) obtained from the subject.

### SEQUENCE LISTING

The instant application contains a Sequence Listing which has been submitted electronically in XML format and is hereby incorporated by reference in its entirety. Said XML copy, created on Dec. 24, 2024, is named 063694-8011US01-seq1 and is 63,686 bytes in size.

### FIELD OF THE INVENTION

The present disclosure generally relates to combination therapy involving a Claudin 18.2 (CLDN18.2) antagonist and a PD-1/PD-L1 axis inhibitor for CLDN18.2-associated diseases.

### BACKGROUND

The Claudin-18 (CLDN18) molecule splice variant 2 (CLDN18A2 or CLDN18.2): NM_001002026, NP_001002026) is an integral transmembrane protein with a molecular weight of approximately 27.72 kD. For healthy cells, CLDN18.2 is only expressed on gastric cells. Most importantly, CLDN18.2 expression is restricted to the differentiated short-lived cells of stomach epithelium, but devoid from the gastric stem cell region. Using sensitive RT-PCR, CLDN18.2 is not detectable in any other normal human organ. CLDN18.2 is highly expressed in several cancer types including stomach, esophageal, pancreatic and lung tumors as well as human cancer cell lines (see Matsuda Y, Semba S, Ueda J, et al. Gastric and intestinal claudin expression at the invasive front of gastric carcinoma[J]. Cancer science, 2007, 98(7): 1014-1019.).

CLDN18.2-expressing cancers may be treated by combination therapy of antibodies targeting CLDN18.2 and immune checkpoint inhibitors, such as PD-1/PD-L1 axis inhibitors, as disclosed, for example, in WO 2021/025177, disclosure of which has been incorporated by reference in its entirety. However, such combination therapy still faces challenges in clinical trials in achieving satisfying therapeutic efficacy.

Therefore, significant need exists for improved combination therapies for CLDN18.2-expressing cancers to meet clinical needs.

### BRIEF SUMMARY OF THE INVENTION

Throughout the present disclosure, the articles “a,” “an,” and “the” are used herein to refer to one or to more than one (i.e., to at least one) of the grammatical object of the article. By way of example, “an antibody” means one antibody or more than one antibody.

The present disclosure provides, among others, a method of treating a CLDN18.2-associated disease or condition in a subject in need thereof, comprising:
administering to the subject a therapeutically effective amount of a CLDN18.2 antagonist in combination with a therapeutically effective amount of PD-1/PD-L1 axis inhibitor,
wherein the subject is determined to have CLDN18.2 expression in a diseased tissue.

In certain embodiments, the method further comprises administering to the subject a therapeutically effective amount of a chemotherapeutic agent.

In certain embodiments, the CLDN18.2 expression in the diseased tissue is higher than or comparable to expression in healthy or noncancerous stomach cells or stomach tissue.

In certain embodiments, the CLDN18.2 expression in the diseased tissue is lower than expression in healthy or noncancerous stomach cells or stomach tissue but higher than expression in a healthy or noncancerous tissue or organ other than stomach.

In certain embodiments, the CLDN18.2 expression in the diseased tissue is comparable to expression in a healthy or noncancerous tissue or organ other than stomach, and is detectable by an anti-CLDN18.2 diagnostic antibody.

In certain embodiments, the expression of CLDN18.2 is on cell surface or is membrane-bound.

In certain embodiments, the subject is determined to have PD-L1 expression in the diseased tissue.

In certain embodiments, the subject is determined to have low or no PD-L1 expression in the diseased tissue.

In certain embodiments, the PD-L1 expression in the diseased tissue is lower than a reference level.

In certain embodiments, no more than 20% of the cells of the diseased tissue are positive for PD-L1 expression.

In certain embodiments, the cells of a diseased tissue comprise disease cells and immune cells in the diseased tissue.

In certain embodiments, the PD-L1 expression in the diseased tissue is comparable to that in a healthy or noncancerous tissue.

In certain embodiments, the PD-L1 expression in the diseased tissue is low or non-detectable by an anti-PD-L1 diagnostic antibody.

In another aspect, the present disclosure also provides a method of sensitizing a disease or condition to a treatment with PD-1/PD-L1 axis inhibitor in a subject in need thereof, wherein the subject is determined to have low or no expression of PD-L1 in a diseased tissue, the method comprising:
a) determining presence or expression level of CLDN18.2 in the diseased tissue obtained from the subject; and
b) when the presence of CLDN18.2 is determined in step a) or when the expression level of CLDN18.2 reaches a threshold level in step a), administering to the subject a therapeutically effective amount of a CLDN18.2 antagonist, optionally in combination with a therapeutically effective amount of a PD-1/PD-L1 axis inhibitor,
thereby sensitizing the disease or condition to a treatment with PD-1/PD-L1 axis inhibitor.

In certain embodiments, the step b) further comprises administering to the subject a chemotherapeutic agent.

In certain embodiments, the expression of CLDN18.2 is on cell surface or is membrane-bound.

In certain embodiments, the PD-L1 expression in the diseased tissue is lower than a reference level.

In certain embodiments, no more than 20% of the cells of the diseased tissue are positive for PD-L1 expression.

In certain embodiments, the cells of a diseased tissue comprise disease cells and immune cells in the diseased tissue.

In certain embodiments, the PD-L1 expression in the diseased tissue is comparable to that in a healthy or noncancerous tissue.

In certain embodiments, the PD-L1 expression in the diseased tissue is low or non-detectable by an anti-PD-L1 diagnostic antibody.

In another aspect, the present disclosure also provides a method for increasing responsiveness of a tumor to a treatment with PD-1/PD-L1 axis inhibitor in a subject, wherein the subject is determined to have a tumor resistant or refractory to the treatment with a PD-1/PD-L1 axis inhibitor, comprising:
a) determining presence or expression level of CLDN18.2 in a tumor sample obtained from the subject; and
b) when the presence of CLDN18.2 is determined in step a) or when the expression level of CLDN18.2 reaches a threshold level in step a), administering to the subject a therapeutically effective amount of a CLDN18.2 antagonist, and optionally in combination with a therapeutically effective amount of a PD-1/PD-L1 axis inhibitor,
thereby increasing responsiveness of the tumor to the treatment with PD-1/PD-L1 axis inhibitor in the subject.

In certain embodiments, the step b) further comprises administering to the subject a chemotherapeutic agent.

In certain embodiments, the expression of CLDN18.2 is on cell surface or is membrane-bound.

In certain embodiments, the PD-L1 expression in a tumor tissue is lower than a reference level.

In certain embodiments, no more than 20% of the cells of a tumor tissue are positive for PD-L1 expression.

In certain embodiments, the cells of the tumor tissue comprise cancer cells and immune cells in the tumor tissue.

In certain embodiments, the PD-L1 expression in a tumor tissue is comparable to that in a healthy or noncancerous tissue.

In certain embodiments, the PD-L1 expression in a tumor tissue is low or non-detectable by an anti-PD-L1 diagnostic antibody.

In another aspect, the present disclosure also provides a method for determining the eligibility for or likelihood of responsiveness to treatment with a CLDN18.2 antagonist, optionally in combination with a PD-1/PD-L1 axis inhibitor, of a subject having low or no expression level of PD-L1, the method comprising:
a) contacting a sample obtained from the subject with a CLDN18.2 diagnostic agent under conditions that allow detection of expression of the CLDN18.2;
b) determining presence or expression level of CLDN18.2 in the sample;
wherein the subject is determined as eligible for or likely to respond to treatment with a CLDN18.2 antagonist, optionally in combination with a PD-1/PD-L1 axis inhibitor, when the sample has positive expression of CLDN18.2 in the sample, or
wherein the subject is determined as not eligible for or likely to respond to treatment with a CLDN18.2 antagonist, optionally in combination with a PD-1/PD-L1 axis inhibitor, when the expression of CLDN18.2 on the sample is not detected.

In certain embodiments, the CLDN18.2 diagnostic agent is an anti-CLDN18.2 diagnostic antibody.

In certain embodiments, the CLDN18.2 antagonist comprises an anti-CLDN18.2 antibody, such as a monoclonal anti-CLDN18.2 antibody, a bi-specific antibody targeting CLDN18.2 and a second antigen (e.g., CD3, 4-1BB, TGFβ, SIRPα, and IL15), or immune cells expressing chimeric antigen receptors (CARs) or genetically modified TCRs comprising an anti-CLDN18.2 antigen binding domain. In certain embodiments, the anti-CLDN18.2 antibody comprises heavy chain HCDR1, HCDR2 and HCDR3 and/or light chain LCDR1, LCDR2 and LCDR3 sequences, wherein:
the HCDR1 sequence comprises GYNMN (SEQ ID NO: 1), or a homologue sequence of at least 80% sequence identity thereof;
the HCDR2 sequence comprises NIDPYYGGTSYNQKFKG (SEQ ID NO: 2), or a homologue sequence of at least 80% sequence identity thereof;
the HCDR3 sequence comprises MYHGNAFDY (SEQ ID NO: 3), or a homologue sequence of at least 80% sequence identity thereof,
the LCDR1 sequence comprises KSSQSLLNSGNLKNYLT (SEQ ID NO: 4) or a homologue sequence of at least 80% sequence identity thereof;
the LCDR2 sequence comprises WASTRKS (SEQ ID NO: 5) or a homologue sequence of at least 80% sequence identity thereof,
the LCDR3 sequence comprises QNDYSYPLT (SEQ ID NO: 6) or a homologue sequence of at least 80% sequence identity thereof.

In certain embodiments, the anti-CLDN18.2 antibody comprises a heavy chain variable region and a light chain variable region, wherein
the heavy chain variable region comprises an amino acid sequence of SEQ ID NO: 7, and the light chain variable region comprises an amino acid sequence of SEQ ID NO: 8.

In certain embodiments, the anti-CLDN18.2 antibody further comprises an immunoglobulin constant region, optionally a constant region of human Ig, or optionally a constant region of human IgG.

In certain embodiments, the anti-CLDN18.2 antibody further comprises a constant region of human IgG1, IgG2, IgG3, or IgG4.

In certain embodiments, the constant region of human IgG1 comprises SEQ ID NO: 9, or a homologous sequence having at least 80% sequence identity thereof.

In certain embodiments, the constant region comprises one or more amino acid residue substitutions or modifications conferring increased CDC or ADCC relative to wild-type constant region.

In certain embodiments, the constant region comprises one or more amino acid residue substitutions relative to SEQ ID NO: 9, selected from the group consisting of: L235V, F243L, R292P, Y300L, P396L, or any combination thereof.

In certain embodiments, the constant region comprises the sequence of SEQ ID NO: 11.

In certain embodiments, the constant region further comprises the sequence of SEQ ID NO: 10.

In certain embodiments, the anti-CLDN18.2 antibody is humanized.

In certain embodiments, the anti-CLDN18.2 antibody comprises a heavy chain variable region and a light chain variable region, wherein
the heavy chain variable region comprises an amino acid sequence selected from the group consisting of SEQ ID NO: 12, SEQ ID NO: 13 and SEQ ID NO: 14, and
the light chain variable region comprises an amino acid sequence selected from the group consisting of SEQ ID NO: 15 and SEQ ID NO: 16.

In certain embodiments, the anti-CLDN18.2 antibody comprises a heavy chain and a light chain, wherein
the heavy chain comprises an amino acid sequence of SEQ ID NO: 39, and
the light chain comprises an amino acid sequence of SEQ ID NO: 40.

In certain embodiments, the anti-CLDN18.2 antibody is capable of inducing the expression of PD-L1 in the diseased tissue of the subject.

In certain embodiments, the anti-CLDN18.2 antibody is linked to one or more conjugate moieties.

In certain embodiments, the conjugate moiety comprises a clearance-modifying agent, a chemotherapeutic agent, a toxin, a radioactive isotope, a lanthanide, a luminescent label, a fluorescent label, an enzyme-substrate label, a DNA-alkylators, a topoisomerase inhibitor, a tubulin-binders, a cytokine (e.g., IL-15, IL-2, IL-7), or other anticancer drugs.

In certain embodiments, the PD-1/PD-L1 axis inhibitor comprises PD-1 inhibitor selected from the group consisting of antibody, small molecule, and combination thereof.

In certain embodiments, the PD-1 inhibitor comprises an anti-PD-1 antibody selected from the group consisting of: Nivolumab (OPDIVO; BMS-936558), Dostarlimab (TSR-042), Pembrolizumab (KEYTRUDA; MK-3475), MEDI0680 (AMP-514), MEDI4736, BI 754091, Pidilizumab (CT-011), Cemiplimab (LIBTAYO, REGN2810), Spartalizumab (PDR001), Cetrelimab (JNJ 63723283), Toripalimab (JS001), PF-06801591, Tislelizumab (BGB-A317), AMP-224 (GSK-2661380), ABBV-181, Lambrolizumab, Camrelizuma (SHR-1210), Sintilimab (Tyvyt, IBI308), Penpulimab(AK105), Zimberelimab, Retifanlimab, Serplulimab, Balstilimab, Geptanolimab, Prolgolimab, Ezabenlimab, Sasanlimab, Pimivalimab, Budigalimab, Nofazinlimab, Sindelizumab, MGA404, Sym021, BAT1306, HX008.

In certain embodiments, the PD-1/PD-L1 axis inhibitor comprises PD-L1 inhibitor selected from the group consisting of antibody, small molecule, and combination thereof.

In certain embodiments, the PD-L1 inhibitor comprises an anti-PD-L1 antibody selected from the group consisting of: Atezolizumab (TECENTRIQ; R05541267; MPDL3280A; RG7446), BMS-936559, Avelumab (bavencio), lodapolimab (LY3300054), Durvalumab (MEDI4736), CX-072 (Proclaim-CX-072), FAZ053, Envafolimab(KN035), MDX-1105, STI-1040, CS1001, Adebrelimab (SHR-1316), SHR-1701, TOB2450, Bintrafusp, LP002, STI-3031, Cosibelimab, Pacmilimab, NM01, LDP, AMP-224, Garivulimab(BGB-A333), A167,SCD-135, Opucolimab, GR1405.

In certain embodiments, the PD-L1 inhibitor comprises a bispecific antibody targeting both PD-L1 and another checkpoint molecule selected from the group consisting of PD-1, PD-L1, PD-L2, CLTA-4, STRP, TIM-3, LAG3, A2AR, CD160, 2B4, TGFβ, VISTA, BTLA, TIGIT, LAIR1, OX40, CD2, CD27, CD28, CD30, CD40, CD122, ICAM-1, IDO, NKG2C, SLAMF7, SIGLEC7, NKp80, CD160, B7-H3, LFA-1, 1COS, 4-1BB, GITR, BAFFR, HVEM, CD7, LIGHT, IL-2, IL-15, CD3, CD16 or CD83.

In certain embodiments, the checkpoint molecule is TGFβ, 4-1BB, CTLA4, LAG3 or TIGIT.

In certain embodiments, the PD-L1 inhibitor comprises an anti-PD-L1 antibody comprising heavy chain HCDR1, HCDR2 and HCDR3 and/or light chain LCDR1, LCDR2 and LCDR3 sequences, wherein:
the HCDR1 sequence comprises DYYMN (SEQ ID NO: 22), or a homologue sequence of at least 80% sequence identity thereof;
the HCDR2 sequence comprises DINPNNAETLYNHKFKG (SEQ ID NO: 23), or a homologue sequence of at least 80% sequence identity thereof,
the HCDR3 sequence comprises WGDGPFAY (SEQ ID NO: 24), or a homologue sequence of at least 80% sequence identity thereof,
the LCDR1 sequence comprises KASQNVGAAVA (SEQ ID NO: 25) or a homologue sequence of at least 80% sequence identity thereof,
the LCDR2 sequence comprises SVSDRYT (SEQ ID NO: 26) or a homologue sequence of at least 80% sequence identity thereof,
the LCDR3 sequence comprises QQYSNYPT (SEQ ID NO: 27) or a homologue sequence of at least 80% sequence identity thereof.

In certain embodiments, the PD-L1 inhibitor comprises an anti-PD-L1 antibody comprising a heavy chain variable region and a light chain variable region, wherein
the heavy chain variable region comprises an amino acid sequence of SEQ ID NO: 17, and
the light chain variable region comprises an amino acid sequence of SEQ ID NO: 18.

In certain embodiments, the PD-L1 inhibitor comprises an anti-PD-L1 antibody comprising a heavy chain and a light chain, wherein
the heavy chain comprises an amino acid sequence of SEQ ID NO: 19 or SEQ ID NO: 20, and
the light chain comprises an amino acid sequence of SEQ ID NO: 21.

In certain embodiments, the PD-L1 inhibitor comprises an anti-PD-L1 antibody comprising heavy chain HCDR1, HCDR2 and HCDR3 and/or light chain LCDR1, LCDR2 and LCDR3 sequences, wherein:
the HCDR1 sequence comprises TYWMH (SEQ ID NO: 32), or a homologue sequence of at least 80% sequence identity thereof,
the HCDR2 sequence comprises MIQPNSGGTKYNEKFKK (SEQ ID NO: 33), or a homologue sequence of at least 80% sequence identity thereof;
the HCDR3 sequence comprises GAGTVDYFDY (SEQ ID NO: 34), or a homologue sequence of at least 80% sequence identity thereof,
the LCDR1 sequence comprises RASESVDIYGNSFMH (SEQ ID NO: 35) or a homologue sequence of at least 80% sequence identity thereof,
the LCDR2 sequence comprises RASNLES (SEQ ID NO: 36) or a homologue sequence of at least 80% sequence identity thereof,
the LCDR3 sequence comprises QQSTEDPYT (SEQ ID NO: 37) or a homologue sequence of at least 80% sequence identity thereof.

In certain embodiments, the PD-L1 inhibitor comprises an anti-PD-L1 antibody comprising a heavy chain variable region and a light chain variable region, wherein
the heavy chain variable region comprises an amino acid sequence of SEQ ID NO: 28, and
the light chain variable region comprises an amino acid sequence of SEQ ID NO: 29.

In certain embodiments, the PD-L1 inhibitor comprises an anti-PD-L1 antibody comprising a heavy chain and a light chain, wherein
the heavy chain comprises an amino acid sequence of SEQ ID NO. 30, and
the light chain comprises an amino acid sequence of SEQ ID NO. 31.

In certain embodiments, the subject is human.

In certain embodiments, the administration is via oral, nasal, intravenous, subcutaneous, sublingual, or intramuscular administration.

In certain embodiments, the administration of the composition comprising anti-CLDN18.2 antibody is prior to, simultaneously with, or after the administration of the composition comprising PD-1/PD-L1 axis inhibitor.

In certain embodiments, the disease or condition is cancer.

In certain embodiments, the diseased tissue comprises a cancer cell.

In certain embodiments, the cancer is selected from the group consisting of gastric cancer, lung cancer, bronchial cancer, bone cancer, liver and bile duct cancer, pancreatic cancer, breast cancer, liver cancer, ovarian cancer, testicle cancer, kidney cancer, bladder cancer, head and neck cancer, spine cancer, brain cancer, cervix cancer, uterine cancer, endometrial cancer, colon cancer, colorectal cancer, rectal cancer, anal cancer, esophageal cancer, gastrointestinal cancer, skin cancer, prostate cancer, pituitary cancer, stomach cancer, vagina cancer, thyroid cancer, glioblastoma, astrocytoma, melanoma, myelodysplastic syndrome, sarcoma, teratoma, and adenocarcinoma.

In certain embodiments, the cancer is gastric cancer, lung cancer, colon cancer, bile duct cancer, or combination thereof.

In certain embodiments, the disease or condition is resistant or refractory to a treatment with a PD-1/PD-L1 axis inhibitor.

In certain embodiments, the resistance is de novo or acquired.

In certain embodiments, the disease or condition is further resistant or refractory to a second therapy selected from the group consisting of chemotherapy, radiotherapy, immunotherapy, and combination thereof.

In certain embodiments, the disease or condition is resistant or refractory to a combinatory therapy with a PD-1/PD-L1 axis inhibitor and a chemotherapeutic agent.

In certain embodiments, the chemotherapeutic agent is selected from the group consisting of: antimetabolites such as methotrexate and 5-fluorouracil (5-FU), Oxaliplatin, alkylating agents (e.g., thiotepa and cyclophosphamide (Cytoxan™), alkyl sulfonates (e.g., busulfan, improsulfan and piposulfan), aziridines (e.g., benzodopa, carboquone, meturedopa, and uredopa), emylerumines and memylamelamines (e.g., altretamine, triemylenemelamine, triethylenephosphoramide, triethylenethiophosphoramide, and trimemylolomelamine), acetogenins, a camptothecin (e.g., synthetic analogue topotecan), bryostatin, callystatin, CC-1065 (including its adozelesin, carzelesin and bizelesin synthetic analogues), cryptophycins (articularly cryptophycin and cryptophycin), dolastatin, duocarmycin (including the synthetic analogues, KW-2 189 and CBI-TMI), eleutherobin, pancratistatin, a sarcodictyin, spongistatin, cisplatin, nitrogen mustards (e.g., chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard), nitrosoureas (e.g., carmustine, chlorozotocin, foremustine, lomustine, nimustine, ranimustine, folic acid analogues (e.g., demopterin, methotrexate, pteropterin, trimetrexate, purine analogs (e.g., fludarabine, 6-mercaptopurine, thiamiprine, thioguanine, pyrimidine analogues (e.g., ancitabine, azacitidine, 6-azauridine, carmofur, cytarabine, dideoxyuridine, doxifluridine, enocitabine, floxuridine), androgens (e.g., calusterone, dromostanolone propionate, epitiostanol, mepitiostane, testolactone), anti-adrenals (e.g., aminoglutethimide, mitotane, trilostane), folic acid replinisher (e.g., frolinic acid), aceglatone, aldophosphamide glycoside, aminolevulinic acid, eniluracil, amsacrine, hestrabucil, bi santrene, edatrexate, defofamine, demecolcine, diaziquone, elformthine, elliptinium acetate, an epothilone, etoglucid, gallium nitrate, hydroxyurea, lentinan, lonidamine, maytansinoids (e.g., maytansine and ansamitocins), mitoguazone, mitoxantrone, mopidamol, nitracrine, pentostatin, phenamet, pirarubicin, losoxantrone, podophyllinic acid, 2-ethylhydrazide, procarbazine, P SK™, razoxane, rhizoxin, sizofiran, spirogermanium, tenuazonic acid, triaziquone, 2,2,2″-tricUorotriemylamine, trichothecenes, urethane, vindesine, dacarbazine, mannomustine, mitobronitol, mitolactol.

In another aspect, the present disclosure also provides a kit useful in treating a disease or condition in a subject in need thereof, comprising a first container that comprises a CLDN18.2 antagonist and a second container that comprises a PD-1/PD-L1 axis inhibitor, and optionally instructions for use of the kit,
wherein the disease or condition is characterized in having: a) CLDN18.2 expression in a diseased tissue, and/or b) low or no expression of PD-L1 in the diseased tissue.

In another aspect, the present disclosure also provides a kit, comprising a CLDN18.2 antagonist and a package insert comprising instructions for using the CLDN18.2 antagonist in combination with a PD-1/PD-L1 axis inhibitor to treat a disease or condition in a subject in need thereof,
wherein the disease or condition is characterized in having: a) CLDN18.2 expression in a diseased tissue, and/or b) low or no expression of PD-L1 in the diseased tissue.

In another aspect, the present disclosure also provides a kit, comprising a PD-1/PD-L1 axis inhibitor and a package insert comprising instructions for using the PD-1/PD-L1 axis inhibitor in combination with a CLDN18.2 antagonist to treat a disease or condition in a subject in need thereof,
wherein the disease or condition is characterized in having: a) CLDN18.2 expression in a diseased tissue, and/or b) low or no expression of PD-L1 in the diseased tissue.

In another aspect, the present disclosure also provides a kit for predicting responsiveness of a subject to treatment with a CLDN 18.2 antagonist in combination with PD-1/PD-L1 axis inhibitor, comprising: one or more reagents for detecting presence of CLDN 18.2 and/or PD-L1 in a biological sample obtained from the subject; or one or more reagents for measuring expression level of CLDN 18.2 and/or PD-L1 in a biological sample obtained from the subject, optionally wherein the biological sample is a tumor tissue.

In another aspect, the present disclosure also provides use of a pharmaceutical composition, comprising a therapeutically effective amount of a) a CLDN18.2 antagonist, b) a PD-1/PD-L1 axis inhibitor, or c) both, and one or more pharmaceutically acceptable carriers, in the manufacture of a medicament for treating a disease or condition in a subject in need thereof,
wherein the disease or condition is characterized in having: a) CLDN18.2 expression in a diseased tissue, and/or b) low or no expression of PD-L1 in the diseased tissue.

### BRIEF DESCRIPTION OF FIGURES

FIG. 1A is bar graph showing PD-L1 levels of NUGC-4 when cocultured with different supernatant.

FIG. 1B is bar graph showing PD-L1 levels of KATOIII when cocultured with different supernatant.

FIG. 1C is bar graph showing PD-L1 levels of SNU620 when cocultured with different supernatant.

FIG. 2 shows tumor volumes of NCI-H460-CLDN18.2 xenograft models with different treatment.

FIG. 3 shows tumor volumes of CT26-CLDN18.2 syngeneic tumor model with different treatment.

FIG. 4 shows tumor volumes of MC38-CLDN18.2 syngeneic tumor model with different treatment.

FIGS. 5A and 5B show tumor volumes of CT26-CLDN18.2 syngeneic tumor model with different treatment.

FIG. 6A shows representative IHC image of CLDN18.2 and corresponding PD-L1 staining on various cancer tissues in clinical samples with different staining intensity.

FIG. 6B shows representative IHC image of CLDN18.2 and corresponding PD-L1 staining on various cancer tissues in Patient-derived Xenograft (PDX) Samples with different staining intensity.

FIG. 7 shows tumor volumes of NUGC4-hCLDN18.2 and human PBMC co-inoculation tumor model with different treatment.

FIG. 8 shows tumor volumes of MFC-hCLDN18.2 syngeneic tumor model with different treatment.

### DETAILED DESCRIPTION OF THE INVENTION

The following description of the disclosure is merely intended to illustrate various embodiments of the disclosure. As such, the specific modifications discussed are not to be construed as limitations on the scope of the disclosure. It will be apparent to one skilled in the art that various equivalents, changes, and modifications may be made without departing from the scope of the disclosure, and it is understood that such equivalent embodiments are to be included herein. All references cited herein, including publications, patents and patent applications are incorporated herein by reference in their entirety.

### Definitions

As used herein, the term “a,” “an,” “the” and similar terms used in the context of the present invention (especially in the context of the claims) are to be construed to cover both the singular and plural unless otherwise indicated herein or clearly contradicted by the context.

The term “CLDN18.2” refers to Claudin-18 splice variant 2 derived from mammals, such as primates (e.g. humans, monkeys) and rodents (e.g. mice). In certain embodiments, CLDN18.2 is human CLDN18.2. Exemplary sequence of human CLDN18.2 includes human CLDN18.2 protein (NCBI Ref Seq No. NP_001002026.1, or SEQ ID NO: 38). Exemplary sequence of CLDN18.2 includes Mus musculus (mouse) CLDN18.2 protein (NCBI Ref Seq No. NP_001181852.1), Macaca fascicularis (crab-eating macaque) CLDN18.2 protein (NCBI Ref Seq No. XP_015300615.1). CLDN18.2 is expressed in a cancer cell. In one embodiment said CLDN18.2 is expressed on the surface of a cancer cell.

As used herein, the term “antagonist” with respect to CLDN18.2 refers to any molecule that partially or completely inhibits, blocks, or neutralizes a biological activity of CLDN18.2. Suitable CLDN18.2 antagonists may include, without limitation, antibodies, antisense oligonucleotides, peptides, and small organic molecules. In certain embodiments, the CLDN18.2 antagonist is an anti-CLDN18.2 antibody.

“Anti-CLDN18.2 antibody” as used herein refers to an antibody that is capable of specific binding to CLDN18.2 (e.g. human or non-human CLDN18.2) with a sufficient affinity, for example, to provide for diagnostic and/or therapeutic use.

The term “antibody” as used herein includes any immunoglobulin, monoclonal antibody, polyclonal antibody, multivalent antibody, bivalent antibody, monovalent antibody, multispecific antibody, bispecific antibody, or antibody variant (e.g., affinity variant, glycosylation variant, cysteine-engineered variant, Fc variants, antigen-binding fragments, antibody drug conjugates) that binds to a specific antigen.

As used herein, a “bispecific” antibody refers to an artificial antibody which has fragments derived from two different monoclonal antibodies and is capable of binding to two different epitopes. The two epitopes may present on the same antigen, or they may present on two different antigens.

The term “antibody drug conjugate” as used herein refers to the linkage of an antibody or an antigen binding fragment thereof with another agent, such as a chemotherapeutic agent, a toxin, an immunotherapeutic agent, an imaging probe, and the like. The linkage can be covalent bonds, or non-covalent interactions such as through electrostatic forces. Various linkers, known in the art, can be employed to form the antibody drug conjugate. Additionally, the antibody drug conjugate can be provided in the form of a fusion protein that may be expressed from a polynucleotide encoding the conjugate.

As used herein, “fusion protein” refers to proteins created through the joining of two or more genes or gene fragments which originally coded for separate proteins (including peptides and polypeptides). Translation of the fusion gene results in a single protein with functional properties derived from each of the original proteins.

As used herein, the term “antigen-binding fragment” refers to an antibody fragment formed from a fragment of an antibody comprising one or more CDRs, or any other antibody portion that binds to an antigen but does not comprise an intact native antibody structure. Examples of antigen-binding fragment include, without limitation, a diabody, a Fab, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a multispecific antibody, a camelized single domain antibody, a nanobody, a domain antibody, and a bivalent domain antibody. An antigen-binding fragment is capable of binding to the same antigen to which the parent antibody binds. In certain embodiments, an antigen-binding fragment may comprise one or more CDRs from a particular human antibody.

“Fab” with regard to an antibody refers to a monovalent antigen-binding fragment of the antibody consisting of a single light chain (both variable and constant regions) bound to the variable region and first constant region of a single heavy chain by a disulfide bond. Fab can be obtained by papain digestion of an antibody at the residues proximal to the N-terminus of the disulfide bond between the heavy chains of the hinge region.

“Fab′” refers to a Fab fragment that includes a portion of the hinge region, which can be obtained by pepsin digestion of an antibody at the residues proximal to the C-terminus of the disulfide bond between the heavy chains of the hinge region and thus is different from Fab in a small number of residues (including one or more cysteines) in the hinge region.

“F(ab′)₂” refers to a dimer of Fab′ that comprises two light chains and part of two heavy chains.

“Fc” with regard to an antibody refers to that portion of the antibody consisting of the second and third constant regions of a first heavy chain bound to the second and third constant regions of a second heavy chain via disulfide bond. IgG and IgM Fc regions contain three heavy chain constant regions (second, third and fourth heavy chain constant regions in each chain). It can be obtained by papain digestion of an antibody. The Fc portion of the antibody is responsible for various effector functions such as ADCC, ADCP and CDC, but does not function in antigen binding.

“Fv” with regard to an antibody refers to the smallest fragment of the antibody to bear the complete antigen binding site. A Fv fragment consists of the variable region of a single light chain bound to the variable region of a single heavy chain. A “dsFv” refers to a disulfide-stabilized Fv fragment that the linkage between the variable region of a single light chain and the variable region of a single heavy chain is a disulfide bond.

“Single-chain Fv antibody” or “scFv” refers to an engineered antibody consisting of a light chain variable region and a heavy chain variable region connected to one another directly or via a peptide linker sequence (Huston J S et al. Proc Natl Acad Sci USA, 85:5879(1988)). A “scFv dimer” refers to a single chain comprising two heavy chain variable regions and two light chain variable regions with a linker. In certain embodiments, an “scFv dimer” is a bivalent diabody or bivalent ScFv (BsFv) comprising V_{H}-V_{L} (linked by a peptide linker) dimerized with another V_{H}-V_{L} moiety such that V_{H}'S of one moiety coordinate with the V_{L}'S of the other moiety and form two binding sites which can target the same antigens (or epitopes) or different antigens (or epitopes). In other embodiments, a “scFv dimer” is a bispecific diabody comprising V_{H1}-V_{L2} (linked by a peptide linker) associated with V_{L1}-V_{H2} (also linked by a peptide linker) such that V_{H1} and V_{L1} coordinate and V_{H2} and V_{L2} coordinate and each coordinated pair has a different antigen specificity.

“Single-chain Fv-Fc antibody” or “scFv-Fc” refers to an engineered antibody consisting of a scFv connected to the Fc region of an antibody.

“Camelized single domain antibody,” “heavy chain antibody,” “nanobody” or “HCAb” refers to an antibody that contains two V_{H} domains and no light chains (Riechmann L. and Muyldermans S., J Immunol Methods. December 10; 231(1-2):25-38 (1999); Muyldermans S., J Biotechnol. June; 74(4):277-302 (2001); WO94/04678; WO94/25591; U.S. Pat. No. 6,005,079). Heavy chain antibodies were originally obtained from Camelidae (camels, dromedaries, and llamas). Although devoid of light chains, camelized antibodies have an authentic antigen-binding repertoire (Hamers-Casterman C. et al., Nature. June 3; 363(6428):446-8 (1993); Nguyen V K. et al. “Heavy-chain antibodies in Camelidae; a case of evolutionary innovation,” Immunogenetics. April; 54(1):39-47 (2002); Nguyen V K. et al. Immunology. May; 109(1):93-101 (2003)). The variable domain of a heavy chain antibody (VHH domain) represents the smallest known antigen-binding unit generated by adaptive immune responses (Koch-Nolte F. et al., FASEB J. November; 21(13):3490-8. Epub 2007 Jun. 15 (2007)). “Diabodies” include small antibody fragments with two antigen-binding sites, wherein the fragments comprise a V_{H} domain connected to a V_{L} domain in a single polypeptide chain (V_{H}-V_{L} or V_{L}-V_{H}) (see, e.g., Holliger P. et al., Proc Natl Acad Sci USA. July 15; 90(14):6444-8 (1993); EP404097; WO93/11161). The two domains on the same chain cannot be paired, because the linker is too short, thus, the domains are forced to pair with the complementary domains of another chain, thereby creating two antigen-binding sites. The antigen-binding sites may target the same or different antigens (or epitopes).

A “domain antibody” refers to an antibody fragment containing only the variable region of a heavy chain or the variable region of a light chain. In certain embodiments, two or more V_{H} domains are covalently joined with a peptide linker to form a bivalent or multivalent domain antibody. The two V_{H} domains of a bivalent domain antibody may target the same or different antigens.

In certain embodiments, a “(dsFv)₂” comprises three peptide chains: two V_{H} moieties linked by a peptide linker and bound by disulfide bridges to two V_{L} moieties.

In certain embodiments, a “bispecific ds diabody” comprises V_{H1}-V_{L2} (linked by a peptide linker) bound to V_{L1}-V_{H2} (also linked by a peptide linker) via a disulfide bridge between V_{H1} and V_{L1}.

In certain embodiments, a “bispecific dsFv” or “dsFv-dsFv′” comprises three peptide chains: a V_{H1}-V_{H2} moiety wherein the heavy chains are bound by a peptide linker (e.g., a long flexible linker) and paired via disulfide bridges to V_{L1} and V_{L2} moieties, respectively. Each disulfide paired heavy and light chain has a different antigen specificity.

The term “humanized” as used herein means that the antibody or antigen-binding fragment comprises CDRs derived from non-human animals, FR regions derived from human, and when applicable, constant regions derived from human. In certain embodiments, the amino acid residues of the variable region framework of the humanized CLDN18.2 antibody are substituted for sequence optimization. In certain embodiments, the variable region framework sequences of the humanized CLDN18.2 antibody chain are at least 65%, 70%, 75%, 80%, 85%, 90%, 95% or 100% identical to the corresponding human variable region framework sequences.

The term “chimeric” as used herein refers to an antibody or antigen-binding fragment that has a portion of heavy and/or light chain derived from one species, and the rest of the heavy and/or light chain derived from a different species. In an illustrative example, a chimeric antibody may comprise a constant region derived from human and a variable region derived from a non-human species, such as from mouse.

“Percent (%) sequence identity” with respect to amino acid sequence (or nucleic acid sequence) is defined as the percentage of amino acid (or nucleic acid) residues in a candidate sequence that are identical to the amino acid (or nucleic acid) residues in a reference sequence, after aligning the sequences and, if necessary, introducing gaps, to achieve the maximum correspondence. Alignment for purposes of determining percent amino acid (or nucleic acid) sequence identity can be achieved, for example, using publicly available tools such as BLASTN, BLASTp (available on the website of U.S. National Center for Biotechnology Information (NCBI), see also, Altschul S. F. et al, J. Mol. Biol., 215:403-410 (1990); Stephen F. et al, Nucleic Acids Res., 25:3389-3402 (1997)), ClustalW2 (available on the website of European Bioinformatics Institute, see also, Higgins D. G. et al, Methods in Enzymology, 266:383-402 (1996); Larkin M. A. et al, Bioinformatics (Oxford, England), 23(21): 2947-8 (2007)), and ALIGN or Megalign (DNASTAR) software. Those skilled in the art may use the default parameters provided by the tool, or may customize the parameters as appropriate for the alignment, such as for example, by selecting a suitable algorithm. In certain embodiments, the non-identical residue positions may differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well known to those of skill in the art. See, e.g., Pearson (1994) Methods Mol. Biol. 24: 307-331, which is herein incorporated by reference.

As used herein, a “homologue sequence” and “homologous sequence” are used interchangeable and refer to polynucleotide sequences (or its complementary strand) or amino acid sequences that have sequences identity of at least 80% (e.g. at least 85%, 88%, 90%, 91%, 92%, 93%, 94%, 95%, 96%, 97%, 98%, 99%) to another sequences when optionally aligned.

An “isolated” substance has been altered by the hand of man from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide is “isolated” if it has been sufficiently separated from the coexisting materials of its natural state so as to exist in a substantially pure state. An isolated “nucleic acid” or “polynucleotide” are used interchangeably and refer to the sequence of an isolated nucleic acid molecule. In certain embodiments, an “isolated antibody or antigen-binding fragment thereof” refers to the antibody or antigen-binding fragments having a purity of at least 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99% as determined by electrophoretic methods (such as SDS-PAGE, isoelectric focusing, capillary electrophoresis), or chromatographic methods (such as ion exchange chromatography or reverse phase HPLC).

The term “subject” includes human and non-human animals. Non-human animals include all vertebrates, e.g., mammals and non-mammals, such as non-human primates, mouse, rat, cat, rabbit, sheep, dog, cow, chickens, amphibians, and reptiles. Except when noted, the terms “patient” or “subject” are used herein interchangeably.

The term “diseased tissue” as used herein broadly encompasses diseased cell (such as cancer cell) and tissue (such as tissue section).

The term “anti-tumor activity” means a reduction in tumor cell proliferation, viability, or metastatic activity. For example, anti-tumor activity can be shown by a decline in growth rate of abnormal cells that arises during therapy or tumor size stability or reduction, or longer survival due to therapy as compared to control without therapy. Such activity can be assessed using accepted in vitro or in vivo tumor models, including but not limited to xenograft models, allograft models, mouse mammary tumor virus (MMTV) models, and other known models known in the art to investigate anti-tumor activity.

“Effector functions” or “antibody effector functions” as used herein refer to biological activities attributable to the binding of Fc region of an antibody to its effectors such as C1 complex and Fc receptor. Exemplary effector functions include: complement dependent cytotoxicity (CDC) induced by interaction of antibodies and C1q on the C1 complex; antibody-dependent cell-mediated cytotoxicity (ADCC) induced by binding of Fc region of an antibody to Fc receptor on an effector cell; and antibody dependent cell mediated phagocytosis (ADCP), where nonspecific cytotoxic cells that express FcTRs recognize bound antibody on a target cell and subsequently cause phagocytosis of the target cell. Effector functions include both those that operate after the binding of an antigen and those that operate independent of antigen binding.

“Treating” or “treatment” of a condition as used herein includes preventing or alleviating a condition, slowing the onset or rate of development of a condition, reducing the risk of developing a condition, preventing or delaying the development of symptoms associated with a condition, reducing or ending symptoms associated with a condition, generating a complete or partial regression of a condition, curing a condition, or some combination thereof.

A “CLDN18.2-associated” disease or condition as used herein refers to any disease or condition caused by, exacerbated by, or otherwise linked to increased or decreased expression or activities of CLDN18.2. In some embodiments, the CLDN18.2-associated disease or condition is, for example, cancer.

“Cancer” as used herein refers to any medical condition characterized by malignant cell growth or neoplasm, abnormal proliferation, infiltration, or metastasis, and includes both solid tumors and non-solid cancers (e.g., hematologic malignancies) such as leukemia. As used herein “solid tumor” refers to a solid mass of neoplastic and/or malignant cells.

The term “pharmaceutically acceptable” indicates that the designated carrier, vehicle, diluent, excipient(s), and/or salt is generally chemically and/or physically compatible with the other ingredients comprising the formulation, and physiologically compatible with the recipient thereof.

“Targeted therapy” is a type of therapy that acts on specific molecules associated with cancer, such as specific proteins that are present in cancer cells but not normal cells or that are more abundant in cancer cells, or the target molecules in the cancer microenvironment that contributes to cancer growth and survival. Targeted therapy targets a therapeutic agent to a tumor, thereby sparing of normal tissue from the effects of the therapeutic agent.

Reference to “about” a value or parameter herein includes (and describes) embodiments that are directed to that value or parameter per se. For example, description referring to “about X” includes description of “X.” Numeric ranges are inclusive of the numbers defining the range. Generally speaking, the term “about” refers to the indicated value of the variable and to all values of the variable that are within the experimental error of the indicated value (e.g. within the 95% confidence interval for the mean) or within 10 percent of the indicated value, whichever is greater. Where the term “about” is used within the context of a time period (years, months, weeks, days etc.), the term “about” means that period of time plus or minus one amount of the next subordinate time period (e.g. about 1 year means 11-13 months; about 6 months means 6 months plus or minus 1 week; about 1 week means 6-8 days; etc.), or within 10 percent of the indicated value, whichever is greater.

### I. Methods of Use

In one aspect, the present disclosure provides a method of treating a CLDN18.2-associated disease or condition in a subject in need thereof. The method may comprise administering to the subject a therapeutically effective amount of a CLDN18.2 antagonist in combination with a therapeutically effective amount of PD-1/PD-L1 axis inhibitor.

In certain embodiments, the subject is determined to have CLDN18.2 expression in a diseased tissue. In certain embodiments, the subject is further determined to have PD-L1 expression in the diseased tissue. In certain embodiments, the subject has been determined to have both CLDN18.2 expression and PD-L1 expression in the diseased tissue.

In certain embodiments, the subject is further determined to have low or no PD-L1 expression in the diseased tissue. In certain embodiments, the subject has been determined to have CLDN18.2 expression and low or no PD-L1 expression in the diseased tissue.

PD-1/PD-L1 axis inhibitors (e.g., PD-1 inhibitor, PD-L1 inhibitor) are a group of immune checkpoint inhibitors that lead to activation, proliferation and/or increase in signaling of T cells and are used as front-line treatment of multiple types of cancers with prominent curative effects. However, it has been found that immune checkpoint inhibitors are beneficial only in a small fraction of patients and have low activity in several cancers, especially for cancers with low PD-L1 expression (see, Darvin et al., 2018, Immune checkpoint inhibitors: recent progress and potential biomarkers. Exp Mol Med 50(12):165). For example, only approximately 20% of the eligible patients can obtain any durable benefit, and many of these patients would ultimately relapse with drug-resistant disease and subsequently experience disease progression (see D. E. Meyers et al., Targeting the PD-1 PD-L1 axis for the treatment of non-small-cell lung cancer. Curr Oncol. 2018 August; 25(4): e324-e334.). Studies have investigated the correlation between tumor PD-L1 expression and therapeutic efficacy and have shown that PD-L1 overexpression is associated with significantly higher objective response rates (ORRs) (Gettinger et al., Overall survival and long-term safety of nivolumab (anti-programmed death 1 antibody, BMS-936558, ONO-4538) in patients with previously treated advanced non-small-cell lung cancer. J Clin Oncol. 2015; 33:2004-12.; Garon et al., Pembrolizumab for the treatment of non-small-cell lung cancer. N Engl J Med. 2015; 372:2018-28. doi: 10.10561NEJMoa1501824.). Publications and reports at the American Society of Clinical Oncology (ASCO) annual meeting in 2021 have also shown that patients having tumors with low PD-L1 expression can less benefit from treatment with PD-1/PD-L1 axis inhibitor.

The present disclosure surprisingly discovered that in tumor tissues expressing CLDN18.2 and optionally having low or no expression of PD-L1, that binding of a CLDN18.2 antagonist (e.g., an anti-CLDN18.2 antibody) to CLDN18.2 expressed on surface of the cells of the tumor tissue could significantly upregulate the PD-L1 expression on the tumor cell.

Based, at least in part, on the discoveries as mentioned above, the present disclosure provides methods of treating a CLDN18.2-associated disease or condition in a subject in need thereof, wherein the subject is, or has been determined to have: a) CLDN18.2 expression in a diseased tissue (e.g., cell of a cancerous tissue or tumor tissue), and b) low or no expression of PD-L1 in the diseased tissue.

The present disclosure further provides methods of sensitizing PD-1/PD-L1 axis inhibitor in a subject determined to have low or no expression of PD-L1 in a diseased tissue, by administering a therapeutically effective amount of a CLDN18.2 antagonist, optionally in combination with a therapeutically effective amount of PD-1/PD-L1 axis inhibitor.

The present disclosure further provides methods of increasing responsiveness of a tumor to a treatment with PD-1/PD-L1 axis inhibitor in a subject having a tumor resistant or refractory to the treatment with a PD-1/PD-L1 axis inhibitor, by administering a therapeutically effective amount of a CLDN18.2 antagonist, optionally in combination with a therapeutically effective amount of PD-1/PD-L1 axis inhibitor.

### A) Methods of Combination Therapy

In one aspect, the present disclosure provides methods of treating a CLDN18.2-associated disease or condition in a subject in need thereof. In certain embodiments, the methods provided herein comprise administering to the subject a therapeutically effective amount of a CLDN18.2 antagonist in combination with a therapeutically effective amount of PD-1/PD-L1 axis inhibitor.

In certain embodiments, the subject is determined to have CLDN18.2 expression in a diseased tissue. In certain embodiments, the subject is determined to have both CLDN18.2 expression and PD-1 expression in the diseased tissue. In certain embodiments, the subject is determined to have no or low PD-L1 expression in the diseased tissue.

In certain embodiments, the methods provided herein further include a step of selecting a subgroup of patients who have expression of CLDN18.2 but are low or absent in PD-L1 expression in the disease tissue. In certain embodiments, the selected subgroup of patients would be eligible for the combination therapy of CLDN18.2 and PD-1/PD-L1 axis inhibitor for treating the disease or condition. In certain embodiments, the disease or condition is cancer, the patients are cancer patients, and the disease tissues are cancerous tissue.

### B) Methods of Sensitizing a Disease to PD-1/PD-L1 Axis Inhibitor

In another aspect, the present disclosure further provides a method of sensitizing a disease or condition (e.g., cancer) to a treatment with PD-1/PD-L1 axis inhibitor in a subject in need thereof, wherein the disease or condition is characterized in having low or no expression of PD-L1 in a diseased tissue (e.g., cancerous tissue or tumor tissue) obtained from the subject.

In certain embodiments, the method includes determining presence or expression level of CLDN18.2 in the diseased tissue obtained from the subject. In certain embodiments, the subject is determined to have CLDN18.2 expression in a diseased tissue.

In certain embodiments, when the presence of CLDN18.2 is determined or when the expression level of CLDN18.2 reaches a threshold level in the previous determination step, such subject is then administered with a therapeutically effective amount of a CLDN18.2 antagonist, thereby sensitizing the disease or condition in such a subject to a treatment with PD-1/PD-L1 axis inhibitor. In certain embodiments, the method further includes administering a therapeutically effective amount of a PD-1/PD-L1 axis inhibitor after the subject has been sensitized to a treatment with PD-1/PD-L1 axis inhibitor, to achieve significantly improved therapeutic efficacy. The PD-1/PD-L1 axis inhibitor can also be administered simultaneously with, prior to, or after the CLDN18.2 antagonist (e.g., anti-CLDN18.2 antibody).

As used herein, the term “threshold level” in connection with CLDN18.2 expression refers to the minimal expression level of CLDN18.2 that is detectable using conventional techniques, such as immunohistochemistry (IHC) and other suitable methods.

### C) Methods of Reducing or Overcoming Resistance of a Disease to PD-1/PD-L1 Axis Inhibitor

In another aspect, the present disclosure also provides a method for increasing responsiveness of a tumor to a treatment with PD-1/PD-L1 axis inhibitor in a subject, wherein the subject is or has been determined to have a tumor resistant or refractory to the treatment with a PD-1/PD-L1 axis inhibitor.

In certain embodiments, the method includes determining presence or expression level of CLDN18.2 in a tumor sample obtained from the subject.

In certain embodiments, when the presence of CLDN18.2 is determined or when the expression level of CLDN18.2 reaches a threshold level in the previous determination step for the subject, such subject is administered with a therapeutically effective amount of a CLDN18.2 antagonist, optionally in combination with a therapeutically effective amount of a PD-1/PD-L1 axis inhibitor, thereby increasing responsiveness of the tumor to the treatment with PD-1/PD-L1 axis inhibitor in the subject.

The resistance to the PD-1/PD-L1 axis inhibitor can be de novo or acquired. As used herein, the term “de novo” with respect to resistance to treatment with a PD-1/PD-L1 axis inhibitor refers to resistance occurred at initial treatment with a PD-1/PD-L1 axis inhibitor, that is, a subject with de novo resistance is not responsive at the first time of receiving PD-1/PD-L1 axis inhibitor treatment. The term “acquired” used with respect to resistance to treatment with a PD-1/PD-L1 axis inhibitor refers to resistance that is absent at initial treatment with a PD-1/PD-L1 axis inhibitor and occurs during the treatment with a PD-1/PD-L1 axis inhibitor, that is, a subject with acquired resistance can respond to treatment with a PD-1/PD-L1 axis inhibitor and become resistant or irresponsive later to treatment with a PD-1/PD-L1 axis inhibitor.

Resistance to treatment with a PD-1/PD-L1 axis inhibitor can be detected by various methods know in the art, for example, by measuring the reduction in tumor volume after treatment with a PD-1/PD-L1 axis inhibitor.

Selection of appropriate patient subgroups in targeted therapies can significantly maximize efficacy, lower costs, and avoid missed treatment opportunities. For example, as mentioned above, cancer patients having low or no PD-L1 expression are often not eligible for treatment with PD-1/PD-L1 axis inhibitors. However, with the step of detecting CLDN18.2 expression in such patients and selecting those patients with CLDN18.2 expression, the selected patients who are otherwise not responsive to treatment with PD-1/PD-L1 axis inhibitors, become eligible for and responsive to treatment with PD-1/PD-L1 axis inhibitors, when in combination with a CLDN18.2 antagonist. On the other hand, with the CLDN18.2 detecting and selecting step as mentioned above, the combination therapy will not be wasted on the patients who have no or undetectable CLDN18.2 expression, such that these patients would have sufficient time to search for more suitable therapies and avoid missed optimal treatment opportunities.

In certain embodiments, the disease or condition is further resistant or refractory to a second therapy selected from the group consisting of chemotherapy, radiotherapy, immunotherapy, and combination thereof.

In certain embodiments, the disease or condition is resistant or refractory to a combinatory therapy with a PD-1/PD-L1 axis inhibitor (e.g., stand of care anti-PD-1 antibodies or anti-PD-L1 antibodies) and chemotherapy (e.g., stand of care chemotherapy agents, such as Oxaliplatin and Fluorouracil (5FU)).

The term “chemotherapy”, as used herein, refers to the treatment of cancer (cancerous or tumor cells) with one or more cytotoxic anti-neoplastic drugs that can also be called as “chemotherapeutic agents” or “chemotherapeutic drugs” as part of a standardized regimen. Exemplary chemotherapeutic agent includes, without limitation, methotrexate and 5-fluorouracil (5-FU), Oxaliplatin, alkylating agents (e.g., thiotepa and cyclophosphamide (Cytoxan™), alkyl sulfonates (e.g., busulfan, improsulfan and piposulfan), aziridines (e.g., benzodopa, carboquone, meturedopa, and uredopa), emylerumines and memylamelamines (e.g., altretamine, triemylenemelamine, triethylenephosphoramide, triethylenethiophosphoramide, and trimemylolomelamine), acetogenins, a camptothecin (e.g., synthetic analogue topotecan), bryostatin, callystatin, CC-1065 (including its adozelesin, carzelesin and bizelesin synthetic analogues), cryptophycins (articularly cryptophycin and cryptophycin), dolastatin, duocarmycin (including the synthetic analogues, KW-2 189 and CBI-TMI), eleutherobin, pancratistatin, a sarcodictyin, spongistatin, cisplatin, nitrogen mustards (e.g., chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard), nitrosoureas (e.g., carmustine, chlorozotocin, foremustine, lomustine, nimustine, ranimustine, folic acid analogues (e.g., demopterin, methotrexate, pteropterin, trimetrexate, purine analogs (e.g., fludarabine, 6-mercaptopurine, thiamiprine, thioguanine, pyrimidine analogues (e.g., ancitabine, azacitidine, 6-azauridine, carmofur, cytarabine, dideoxyuridine, doxifluridine, enocitabine, floxuridine), androgens (e.g., calusterone, dromostanolone propionate, epitiostanol, mepitiostane, testolactone), anti-adrenals (e.g., aminoglutethimide, mitotane, trilostane), folic acid replinisher (e.g., frolinic acid), aceglatone, aldophosphamide glycoside, aminolevulinic acid, eniluracil, amsacrine, hestrabucil, bi santrene, edatrexate, defofamine, demecolcine, diaziquone, elformthine, elliptinium acetate, an epothilone, etoglucid, gallium nitrate, hydroxyurea, lentinan, lonidamine, maytansinoids (e.g., maytansine and ansamitocins), mitoguazone, mitoxantrone, mopidamol, nitracrine, pentostatin, phenamet, pirarubicin, losoxantrone, podophyllinic acid, 2-ethylhydrazide, procarbazine, P SK™, razoxane, rhizoxin, sizofiran, spirogermanium, tenuazonic acid, triaziquone, 2,2,2″-tricUorotriemylamine, trichothecenes, urethane, vindesine, dacarbazine, mannomustine, mitobronitol, mitolactol. In certain embodiments, the chemotherapy or chemotherapeutic agent is Oxaliplatin and/or 5FU.

Methods of Determining the Eligibility for or Likelihood of Responsiveness to Treatment with a CLDN18.2 Antagonist

Based, at least in part, on the discovery of the present disclosure as mentioned above, CLDN18.2 can be considered as a biomarker whose presence is predictive of responsiveness of a subject to a combination therapy of a CLDN18.2 antagonist and a treatment with PD-1/PD-L1 axis inhibitor. In certain embodiments, the subject has a tumor resistant or refractory to the treatment with a PD-1/PD-L1 axis inhibitor, and optionally with a stand care chemotherapy. The resistance can be de novo or acquired.

In another aspect, the present disclosure also provides a method for determining the eligibility of a subject having low or no expression level of PD-L1 for a combination therapy with a CLDN18.2 antagonist and a PD-1/PD-L1 axis inhibitor or the likelihood of responsiveness of the subject to a combination therapy with a CLDN18.2 antagonist and a PD-1/PD-L1 axis inhibitor.

In certain embodiments, the method includes contacting a sample obtained from the subject with a CLDN18.2 diagnostic agent (e.g., an anti-CLDN18.2 diagnostic antibody) under conditions that allow detection of expression of the CLDN18.2. When the sample has positive expression of CLDN18.2 in a cell of the sample, the subject is determined as eligible for or likely to respond to combination therapy with a CLDN18.2 antagonist and a PD-1/PD-L1 axis inhibitor; when the expression of CLDN18.2 on the sample is not detected, the subject is determined as not eligible for or likely to respond to treatment with a CLDN18.2 antagonist.

In certain embodiments, the subject is identified as likely to respond to the combination therapy of CLDN 18.2 antagonist and PD-1/PD-L1 axis inhibitor based on having expression of CLDN18.2 in the diseased tissue, and optionally further having low or no PD-L1 expression in the diseased tissue.

PD-L1 Expression and/or CLDN18.2 Expression Determination

The methods of uses provided herein involves determination of expression of CLDN18.2 and/or PD-L1. In certain embodiments, the subject is determined to have CLDN18.2 expression in a diseased tissue. In certain embodiments, the subject is determined to have both CLDN18.2 expression and PD-1 expression in the diseased tissue. In certain embodiments, the subject is determined to have no or low PD-L1 expression in the diseased tissue.

Suitable methods known in the art can be used and are described in detail below.

i. Sample Preparation

In certain embodiments, the subject is human. In certain embodiments, the method provided herein further comprises providing a biological sample from the subject, wherein the biological sample comprises the diseased tissue (e.g., cancerous tissue or tumor tissue).

Any biological sample suitable for conducting the methods provided herein can be obtained from the subject. As used herein, “biological sample” refers to a biological specimen taken by sampling from a subject, optionally with additional processing. The collection of a sample from a subject is performed in accordance with the standard protocol generally followed by hospital or clinics, such as during a biopsy.

In certain embodiments, the sample can be a biological sample comprising cancer cells, or non-cancer cells. For example, non-cancer cells can be from the same tissue or organ as the cancer cells are also found. In certain embodiments, the biological sample containing or suspected of containing a cancer cell can be obtained from the subject. In some embodiments, the biological sample can be derived from a cancer cell or cancer tissue, or tumor infiltrating immune cells. In certain embodiment, a biological sample is a tumor tissue.

In some embodiments, the biological sample is a fresh or archived sample obtained from a tumor tissue, e.g., by a tumor biopsy or fine needle aspirate. In some embodiments, the sample can be any biological fluid containing cancer cells or non-cancer cells (e.g. peripheral blood mononuclear cells (PBMC)).

Examples of a biological sample include without limitation, bodily fluid, such as blood, plasma, serum, urine, vaginal fluid, uterine or vaginal flushing fluids, pleural fluid, ascetic fluid, cerebrospinal fluid, saliva, sweat, tears, sputum, bronchioalveolar lavage fluid, etc., and tissues, such as biopsy tissue (e.g. biopsied bone tissue, bone marrow, breast tissue, gastrointestinal tract tissue, lung tissue, colon tissue, liver tissue, prostate tissue, brain tissue, nerve tissue, meningeal tissue, colon tissue, renal tissue, endometrial tissue, cervical tissue, lymph node tissue, muscle tissue, or skin tissue), a paraffin embedded tissue. In a further embodiment, a biological sample comprises cells, tissue, blood, plasma, serum, urine, mouthwash, stool, saliva, and any combination thereof.

In certain embodiments, the sample can be further processed by a desirable method for determining expression level of the at least one biomarker, such as CLDN18.2 and/or PD-L1.

ii. Determination of CLDN18.2 Expression

In certain embodiments, CLDN18.2 expression is determined from the diseased tissue (e.g. from the biological sample).

In some embodiments, the CLDN18.2 expression in the diseased tissue (e.g., cancerous tissue or tumor tissue), is higher than or comparable to expression in healthy or noncancerous stomach cells or stomach tissue.

In some embodiments, the CLDN18.2 expression in the diseased tissue (e.g., cancerous tissue or tumor tissue), is lower than expression in healthy or noncancerous stomach cells or stomach tissue but higher than expression in a healthy or noncancerous tissue or organ other than stomach.

In some embodiments, the CLDN18.2 expression in the diseased tissue (e.g., cancerous tissue or tumor tissue), is comparable to expression in a healthy or noncancerous tissue or organ other than stomach and is detectable by an anti-CLDN18.2 diagnostic antibody.

In some embodiments, the expression of CLDN18.2 is on cell surface or is membrane-bound.

The presence and/or expression level of CLDN18.2 in the diseased tissue (e.g., cancerous tissue or tumor tissue), can be determined by various methods known in the art. In certain embodiments, the biological sample may be further processed to, for example, isolate the analyte such as the nucleic acids or proteins. Presence and/or expression level of CLDN18.2 can be determined by, for example, quantitative fluorescence cytometry, immunohistochemistry (IHC), or nucleic acid-based methods. For example, the biological sample from the subject can be exposed to anti-CLDN18.2 antibody or antigen-binding fragment thereof, which binds to and detects the expressed CLDN18.2 protein.

In certain embodiments, the expression of CLDN18.2 in the diseased tissue (e.g., cancerous tissue or tumor tissue), is determined or measured by IHC. In certain embodiments, the expression level of human CLDN18.2 protein on a cancerous tissue or tumor tissue from the subject can be determined in accordance to the methods described in Example 6 provided herein.

In certain embodiments, the subject is or has been determined to have high CLDN18.2 expression in the diseased tissue (e.g., cancerous tissue or tumor tissue), derived from the subject. In certain embodiments, the diseased tissue is or has been determined to have CLDN18.2 expression higher than or comparable to expression in healthy or noncancerous stomach cells or stomach tissue. The high CLDN18.2 expression in a biological sample, such as a diseased tissue (e.g., cancerous tissue or tumor tissue) refers to expression of CLDN18.2 at an intensity of at least 2+ as measured by IHC and at a level where at least 40% (e.g. at least 45%, at least 50%, at least 55%, at least 60%, at least 65%, at least 70%, at least 75%, at least 80%, at least 85%, at least 90%, at least 95%, 40-100%, 50-100%, 60-100%, 70-100%, 80-100%, 90-100%, 40-90%, 50-90%, 60-90%, 70-90%, 80-90%, 40-80%, 40-70%, 40-60%, 40-50%, 50-80%, 50-70%, 50-60%, 60-80%, 60-70%, or 70-80%) of the cells in the biological sample, such as a diseased tissue (e.g., cancerous tissue or tumor tissue), are stained positive in IHC.

In certain embodiments, the subject is or has been determined to have medium CLDN18.2 expression in the diseased tissue (e.g., cancerous tissue or tumor tissue), derived from the subject. In certain embodiments, the diseased tissue is or has been determined to have CLDN18.2 expression lower than expression in healthy or noncancerous stomach cells or stomach tissue but higher than expression in a healthy or noncancerous tissue or organ other than stomach. The medium CLDN18.2 expression in a biological sample, such as a diseased tissue (e.g., cancerous tissue or tumor tissue) refers to expression of CLDN18.2 at an intensity of at least 1+ and below 2+ as measured by IHC and at a level where at least 30% (or at least 35%) but below 40% of the cells are stained positive in IHC.

In certain embodiments, the subject is or has been determined to have low CLDN18.2 expression in the diseased tissue (e.g., cancerous tissue or tumor tissue), derived from the subject. In certain embodiments, the diseased tissue is or has been determined to have CLDN18.2 expression comparable to expression in a healthy or noncancerous tissue or organ other than stomach and is detectable by an anti-CLDN18.2 diagnostic antibody. The low CLDN18.2 expression in a biological sample, such as a diseased tissue (e.g., cancerous tissue or tumor tissue) refers to expression of CLDN18.2 at an intensity of above 0 but below 1+ as measured by IHC and at a level where above 0 but below 30% (e.g. 5%, 10%, 15%, 20%, 25%, 5-25%, 10-25%, 15-25%, 20-25%, 5-20%, 5-15%, 5-10%, 10-20%, or 10-15%) of the cells are stained positive in IHC.

The anti-CLDN18.2 diagnostic antibody can be any anti-CLDN18.2 antibodies that can sensitively detect the expression of CLDN18.2 on a disease cell or a disease tissue. For example, the anti-CLDN18.2 diagnostic antibody can be those described in PCT/CN2021/095411. In certain embodiments, the anti-CLDN18.2 diagnostic antibody is 14G11, which comprises a heavy chain comprising an amino acid sequence of SEQ ID NO: 43, and a light chain comprising an amino acid sequence of SEQ ID NO: 44.

iii. Determination of PD-L1 Expression

In certain embodiments, PD-L1 expression is determined from the diseased tissue (e.g. from the biological sample).

The same biological sample (such as a diseased tissue, e.g., cancerous tissue or tumor tissue) for determination of expression of CLDN18.2, can be further or simultaneously measured to determine if there is expression of PD-L1 on the same sample.

Similar to determining CLDN18.2 expression, the PD-L1 expression in a biological sample, such as a diseased tissue (e.g., cancerous tissue or tumor tissue), can be determined by various methods known in the art. A biological sample containing or suspected of containing a cancer cell can be obtained from the subject. The biological sample can contain cancer cells, cancer tissue, and immune cells (e.g., tumor-associated immune cells, such as tumor infiltrating immune cells). The biological sample may be further processed to, for example, isolate the analyte such as the nucleic acids (e.g., mRNA) or proteins. Presence and/or expression level of PD-L1 can be determined by, for example, quantitative fluorescence cytometry, immunohistochemistry (IHC), or nucleic acid-based methods (e.g., RNA sequencing). For example, the biological sample from the subject can be exposed to anti-PD-L1 antibody or antigen-binding fragment thereof, which binds to and detects the expressed PD-L1 protein. Alternatively, PD-L1 can also be detected at nucleic acid expression level, using methods such as qPCR, reverse transcriptase PCR, microarray, SAGE, FISH, and the like.

In certain embodiments, the PD-L1 expression is determined by IHC. Specific PD-L1 staining by IHC requires appropriate patients' materials, sensitive primary anti-PD-L1 antibodies, and suitable staining protocols. There are mainly three scoring algorithms for PD-L1 staining which are specific to tumor types and clinical decisions: the tumor proportion score (TPS), the immune proportion score (IPS) and the combined positivity score (CPS) (see, Schildhaus et al., Predictive value of PD-L1 diagnostics. Pathologe. 2018 November; 39(6):498-519. doi: 10.1007 s00292-018-0507-x.).

TPS can be defined by the percentage of tumor cells positive for PD-L1 staining in relation to the total tumor cells in a biological sample, detailed description of which can be seen, for example, in Piper et al., Can PD-L1 tumor proportion score be used as the key to unlocking the KEYNOTE studies of pembrolizumab in advanced lung cancer? Transl Lung Cancer Res 2019; 8(5):715-722.

IPS can be defined by the percentage of tumor-associated immune cells positive for PD-L1 expression in relation to the total tumor-associated immune cells, detailed description of which can be seen, for example, in Yang et al., PD-L1 expression on tumor cells and tumor infiltrating immune cells in Chinese colorectal cancer patients. Journal of Clinical Oncology, Volume 38, Issue 15 suppl.

CPS can be defined by the number of PD-L1 staining cells (including, without limitation, tumor cells, lymphocytes, macrophages) divided by the total number of viable tumor cells, multiplied by 100, detailed description can be seen, for example, in Yamashita et al., Prognostic impacts of the combined positive score and the tumor proportion score for programmed death ligand-1 expression by double immunohistochemical staining in patients with advanced gastric cancer. Gastric Cancer. 2020 January; 23(1):95-104; and Dako et al., Development of the combined positive score (CPS)for the evaluation of PD-L1 in solid tumors with the immunohistochemistry assay PD-L1 IHC 22C3 pharmDx. Journal of Clinical Oncology, Volume 35, Issue 15_suppl, 2017. CPS can also be defined by the percentage of PD-L1 staining cells (including, without limitation, tumor cells, lymphocytes, macrophages) in relation to the total number of viable tumor cells, as described in Example 6 of the present disclosure.

CPS for PD-L1 expression on a sample can be obtained using PD-L1 TIC 22C3 pharmDx or PD-L1 TIC 28-8 pharmDx. The PD-L1 IHC 28-8 pharmDx assay (Agilent Technologies, Santa Clara, California, USA), which is a kit for detecting PD-L1 expression, has been approved as a complementary diagnostic for use with nivolumab in cancers, such as non-squamous non-small cell lung cancer (NSQNSCL) and Squamous Cell Carcinoma of the Head and Neck (SCCHN). The PD-L1 IHC 28-8 pharmDx contains optimized reagents and protocol that are required to complete IHC staining of formalin-fixed, paraffin-embedded (FFPE) samples using Autostainer Link 48 and Dako PT Link Pre-treatment Module (Phillips T, Simmons P, Inzunza H D, et al. Development of an automated PD-L1 mmunohistochemistry (IHC) assay for non-small cell lung cancer. Appl Immuno Molec Morph 2015; 23(8):541-9.). In brief, the FFPE samples are first incubated with a primary monoclonal antibody to PD-L1 or the Negative Control Reagent (NCR), followed by incubation with a linker antibody specific to the host species of the primary antibody, and then a ready-to-use visualization reagent including secondary antibody molecules and horseradish peroxidase molecules coupled to a dextran polymer backbone, before being subject to light microscopy for visualization of PD-L1 staining.

The PD-L1 IHC 28-8 pharmDx was used in CheckMate-649, a randomized, multicenter, open-label trial in patients (n=1581) with previously untreated advanced or metastatic gastric cancer, gastroesophageal junction cancer, and esophageal adenocarcinoma. This trial enrolled patients regardless of PD-L1 expression level, and tumor samples from the patients were evaluated using the PD-L1 IHC 28-8 pharmDx assay at a central laboratory. This clinical trial demonstrated statistically significant improvement on overall survival (OS) and progression-free survival (PFS) for patients having tumors with PD-L1 expression of CPS≥5, which was not observed for patients having tumors with PD-L1 expression of CPS lower than 5 (information obtained from https: www.fda.gov media 124784 download).

Different algorithms may be used for different tumor types. For example, TPS can be applied for determining PD-L1 expression in lung cancer, head and neck cancer and melanomas. CPS and IPS are standard measurements of PD-L1 expression in urothelial carcinoma. While for TPS, only PD-L1 staining of tumor cells is regarded as a PD-L1 positive and staining of cells other than tumor cells is not considered as PD-L1 positive; CPS includes PD-L1 expression in tumor-associated immune cells, and IPS is restricted to PD-L1 expression in certain immune cells (e.g., tumor-associated immune cells) (see, Schildhaus et al., Predictive value of PD-L1 diagnostics. Pathologe. 2018 November; 39(6):498-519.).

In certain embodiments, the PD-L1 expression is determined by CPS with the IHC. As described above, CPS can be determined based on the number of PD-L1 positive cells (including, without limitation, tumor, lymphocytes and macrophages) in relation to total tumor cells in a biological sample, and hence allows the capture of PD-L1 expression on tumor and immune cells in a single read. For example, the PD-L1 expression level of 5% (or CPS of 5) in a biological sample, such as a diseased tissue (e.g., cancerous tissue or tumor tissue), means that 5% of the cells (including, without limitation, tumor, lymphocytes and macrophages) in relation to the total tumor cells in the biological sample are positive for PD-L1 staining.

In certain embodiments, in the methods provided herein, the subject is determined to have low or no PD-L1 expression in the cell or in the diseased tissue.

As used herein, the term “low expression of PD-L1” may refer to the PD-L1 expression level that is lower than or no more than a reference level.

In certain embodiments, the term “reference level” with respect to the PD-L1 expression refer to the threshold (e.g., minimal) expression level of PD-L1 in a biological sample, such as a diseased tissue (e.g., cancerous tissue or tumor tissue), derived from a subject who is responsive to the treatment of PD-1/PD-L1 axis inhibitors. Expression of PD-L1 can be measured by methods provided herein. For different tumor types and/or using different PD-L1 detection assays, the threshold expression level of PD-L1 expression could be different. For example, the threshold expression level for gastric cancer can be 5% (or CPS of 5) as measured by the PD-L1 IHC 28-8 pharmDx assay; that is, the methods provided herein are for treating a gastric cancer having PD-L1 expression in the gastric tumor tissue of lower than 5% (or CPS of 5) as measured by PD-L1 IHC 28-8 pharmDx assay and have expression of CLDN18.2 in the gastric tumor tissue. The threshold expression level for gastric cancer may also be 4% (or CPS of 4), 3% (or CPS of 3), 2% (or CPS of 2), or 1% (or CPS of 1) as measured by PD-L1 IHC 28-8 pharmDx assay in some embodiments. In other embodiments, the threshold level for gastric cancer may be 10% (or CPS of 10), 9% (or CPS of 9), 8% (or CPS of 8), 7% (or CPS of 7), 6% (or CPS of 6), 5% (or CPS of 5), 4% (or CPS of 4), 3% (or CPS of 3), 2% (or CPS of 2), or 1% (or CPS of 1), as measured by the PD-L1 IHC 22C3 pharmDx assay, detailed description of which can be seen in, for example, Dako et al., Development of the combined positive score (CPS) for the evaluation of PD-L1 in solid tumors with the immunohistochemistry assay PD-L1 IHC 22C3 pharmDx. Journal of Clinical Oncology, Volume 35, Issue 15_suppl, 2017.

In some embodiments of the methods provided herein, the subject is determined to have PD-L1 expression in the diseased tissue (e.g., cancerous tissue or tumor tissue) that is no more than 20% (or CPS of 20), 15% (or CPS of 15), 10% (or CPS of 10), 9% (or CPS of 9), 8% (or CPS of 8), 7% (or CPS of 7), 6% (or CPS of 6), 5% (or CPS of 5), 4% (or CPS of 4), 3% (or CPS of 3), 2% (or CPS of 2), or 1% (or CPS of 1).

In certain embodiments, the subject is determined to have no more than 20%, no more than 15%, no more than 10%, no more than 9%, no more than 8%, no more than 7%, no more than 6%, no more than 5%, no more than 4%, no more than 3%, no more than 2%, or no more than 1% of the cells of the diseased tissue that are positive for PD-L1 expression, yet still with detectable PD-L1 expression by an anti-PD-L1 diagnostic antibody.

In some embodiments, the subject is determined to have PD-L1 expression in the diseased tissue (e.g., cancerous tissue or tumor tissue) that is comparable to that in a healthy or noncancerous tissue.

As used herein, the term “no expression of PD-L1” means absence of any detectable PD-L1 signal by an anti-PD-L1 diagnostic antibody using various techniques, such as IHC, in a biological sample. In some embodiments, the PD-L1 expression in the diseased tissue is low or non-detectable by an anti-PD-L1 diagnostic antibody.

The reagents used to detect the PD-L1 expression throughout the specification can be an anti-PD-L1 diagnostic antibody, for example, 22C3, as described in US20170285037A1, disclosure of which has been incorporated by reference in its entirety, and a monoclonal rabbit anti-PD-L1, Clone 28-8, which is commercially available.

In some other embodiments of the methods provided herein, the PD-L1 expression in the diseased tissue (e.g., cancerous tissue or tumor tissue), reaches or is comparable or even higher than the reference level.

### Treatment Methods

In certain embodiments, the methods provided herein further comprises administering to the subject a CLDN18.2 antagonist in combination with a PD-1/PD-L1 axis inhibitor. In certain embodiments, the CLDN18.2 antagonist and the PD-1/PD-L1 axis inhibitor are respectively administered at a therapeutically effective amount to the subject. As used herein, the term “therapeutically effective amount” of a CLDN18.2 antagonist or a PD-1/PD-L1 axis inhibitor used in the methods provided herein will depend on various factors known in the art, such as for example body weight, age, past medical history, present medications, state of health of the subject and potential for cross-reaction, allergies, sensitivities and adverse side-effects, as well as the administration route and extent of disease development. Dosages may be proportionally reduced or increased by one of ordinary skill in the art (e.g., physician or veterinarian) as indicated by these and other circumstances or requirements.

In certain embodiments, the CLDN18.2 antagonist and/or PD-1/PD-L1 axis inhibitor may be administered at a therapeutically effective dosage of about 0.01 mg/kg to about 100 mg/kg, about 0.1 mg/kg to about 30 mg/kg, about 1 mg/kg to about 3 mg/kg, about 3 mg/kg to about 30 mg/kg, about 3 mg/kg to about 20 mg/kg, about 6 mg/kg to about 20 mg/kg, about 3 mg/kg to about 10 mg/kg, or about 6 mg/kg to about 10 mg/kg. In certain embodiments, the administration dosage may change over the course of treatment. In certain embodiments, the administration dosage may vary over the course of treatment depending on the reaction of the subject. Dosage regimens may be adjusted to provide the optimum desired response (e.g., a therapeutic response). For example, a single dose may be administered, or several divided doses may be administered over time.

In certain embodiments, the present disclosure provides methods of treating a CLDN18.2-associated disease or condition in a subject who has been identified as likely to respond to the combination therapy treatment of CLDIN 18.2 antagonist and PD-1/PD-L1 axis inhibitor. In certain embodiments, the step of treating comprising administering a therapeutically effective amount of CLDIN 18.2 antagonist and a therapeutically effective amount of PD-1/PD-L1 axis inhibitor to the subject having been identified as likely to respond to the combination therapy treatment of CLDIN 18.2 antagonist and PD-1/PD-L1 axis inhibitor.

The CLDN18.2 antagonist used in the methods provided herein can comprise an anti-CLDN18.2 antibody or antigen-binding fragment thereof. The anti-CLDN18.2 antibody can be a monoclonal antibody, polyclonal antibody, humanized antibody, chimeric antibody, recombinant antibody, bispecific antibody, labeled antibody, bivalent antibody, or anti-idiotypic antibody.

In certain embodiments, the anti-CLDN18.2 antibody has high binding-affinity to a biological sample having high expression of CLDN 18.2.

In certain embodiments, the anti-CLDN18.2 antibody has high binding-affinity to a biological sample having medium expression of CLDN 18.2.

In certain embodiments, the anti-CLDN18.2 antibody has high binding-affinity to a biological sample having low expression of CLDN 18.2.

The high, medium, or low expression of CLDN 18.2 in a biological sample can be defined in the same manner as described above by comparing to a healthy or non-cancerous tissue other than a gastric cancer and a healthy or non-cancerous gastric cancer, or by scoring using results obtained from IHC.

In certain embodiments, the anti-CLDN18.2 antibody comprises heavy chain HCDR1, HCDR2 and HCDR3 and/or light chain LCDR1, LCDR2 and LCDR3 sequences, wherein:
the HCDR1 sequence comprises GYNMN (SEQ ID NO: 1), or a homologue sequence of at least 80% sequence identity thereof;
the HCDR2 sequence comprises NIDPYYGGTSYNQKFKG (SEQ ID NO: 2), or a homologue sequence of at least 80% sequence identity thereof;
the HCDR3 sequence comprises MYHGNAFDY (SEQ ID NO: 3), or a homologue sequence of at least 80% sequence identity thereof,
the LCDR1 sequence comprises KSSQSLLNSGNLKNYLT (SEQ ID NO: 4) or a homologue sequence of at least 80% sequence identity thereof,
the LCDR2 sequence comprises WASTRKS (SEQ ID NO: 5) or a homologue sequence of at least 80% sequence identity thereof;
the LCDR3 sequence comprises QNDYSYPLT (SEQ ID NO: 6) or a homologue sequence of at least 80% sequence identity thereof.

CDRs are known to be responsible for antigen binding, however, it has been found that not all of the 6 CDRs are necessarily indispensable or unchangeable. In other words, it is possible to replace or change or modify 1, 2, or 3 CDRs in the anti-CLDN18.2 antibody, yet substantially retain the specific binding affinity to CLDN18.2.

In certain embodiments, the anti-CLDN18.2 antibody comprises a heavy chain CDR3 sequence of MYHGNAFDY (SEQ ID NO: 21). Heavy chain CDR3 regions are located at the center of the antigen-binding site, and therefore are believed to make the most contact with antigen and provide the most free energy to the affinity of antibody to antigen. It is also believed that the heavy chain CDR3 is by far the most diverse CDR of the antigen-binding site in terms of length, amino acid composition and conformation by multiple diversification mechanisms (Tonegawa S. Nature. 302:575-81). The diversity in the heavy chain CDR3 is sufficient to produce most antibody specificities (Xu J L, Davis M M. Immunity. 13:37-45) as well as desirable antigen-binding affinity (Schier R, etc. J Mol Biol. 263:551-67).

In some embodiments, the anti-CLDN18.2 antibody comprises all or a portion of the heavy chain variable domain and/or all or a portion of the light chain variable domain. In one embodiment, the anti-CLDN18.2 antibody is a single domain antibody which consists of all or a portion of the heavy chain variable domain provided herein. More information of such a single domain antibody is available in the art (see, e.g., U.S. Pat. No. 6,248,516).

In certain embodiments, the anti-CLDN18.2 antibody comprises a heavy chain variable region and a light chain variable region, wherein the heavy chain variable region comprises an amino acid sequence of SEQ ID NO: 7, and the light chain variable region comprises an amino acid sequence of SEQ ID NO: 8.

In certain embodiments, the anti-CLDN18.2 antibody further comprises an immunoglobulin constant region, optionally a constant region of human Ig, or optionally a constant region of human IgG. In certain embodiments, the anti-CLDN18.2 antibody further comprises a constant region of human IgG1, IgG2, IgG3, or IgG4.

In some embodiments, an immunoglobulin constant region comprises a heavy chain and/or a light chain constant region. The heavy chain constant region comprises CH1, hinge, and/or CH2-CH3 regions. In certain embodiments, the heavy chain constant region comprises an Fc region. In certain embodiments, the light chain constant region comprises Cκ or Cλ.

In certain embodiments, the anti-CLDN18.2 antibodies and antigen-binding fragments thereof provided herein comprises a constant region of IgG1 isotype. In certain embodiments, the constant region of human IgG1 comprises SEQ ID NO: 9, or a homologous sequence having at least 80% (e.g. at least 85%, 90%, 95%, 96%, 97%, 98%, or 99%) sequence identity thereof.

Constant region of IgG1 isotype can induce effector functions such as ADCC or CDC. Effector functions of the anti-CLDN18.2 antibody can lead to cytotoxicity to cells expressing CLDN18.2. Effector functions can be evaluated using various assays such as Fc receptor binding assay, C1q binding assay, and cell lysis assay, and any of the assays described above for determining ADCC or CDC.

In certain embodiments, the constant region comprises one or more amino acid residue substitutions or modifications conferring increased CDC or ADCC relative to wild-type constant region.

As used herein, “ADCC” or “antibody-dependent cell-mediated cytotoxicity” refers to a cell-mediated reaction in which nonspecific cytotoxic cells that express Fc receptors (FcRs) (e.g. natural killer (NK) cells, neutrophils, and macrophages) recognize bound antibody on a target cell and subsequently cause lysis of the target cell. Lysis of the target cell is extracellular, requires direct cell-to-cell contact, and does not involve complement. ADCC can be viewed as a mechanism to directly induce a variable degree of immediate tumor destruction that leads to antigen presentation and the induction of tumor-directed T-cell responses. In vivo induction of ADCC is believed to lead to tumor-directed T-cell responses and host-derived antibody responses. In certain embodiments, the constant region comprises one or more amino acid residue substitutions relative to SEQ ID NO: 9, selected from the group consisting of: L235V, F243L, R292P, Y300L, P396L, or any combination thereof. In certain embodiments, the constant region comprises the sequence of SEQ ID NO: 11, and optionally further comprises the sequence of SEQ ID NO: 10.

In certain embodiments, the anti-CLDN18.2 antibody is humanized. A humanized antibody or antigen-binding fragment is desirable in its reduced immunogenicity in human. A humanized antibody is chimeric in its variable regions, as non-human CDR sequences are grafted to human or substantially human FR sequences. Humanization of an antibody or antigen-binding fragment can be essentially performed by substituting the non-human (such as murine) CDR genes for the corresponding human CDR genes in a human immunoglobulin gene (see, for example, Jones et al. (1986) Nature 321:522-525; Riechmann et al. (1988) Nature 332:323-327; Verhoeyen et al. (1988) Science 239:1534-1536). In certain embodiments, the humanized light and heavy chains of the present disclosure are substantially non-immunogenic in humans and retain substantially the same affinity as or even higher affinity than the parent antibody to CLDN18.2.

In certain embodiments, the anti-CLDN18.2 antibody or an antigen-binding fragment thereof provided herein, comprises a heavy chain variable region comprising the sequence selected from the group consisting of SEQ ID NO: 12, SEQ ID NO: 13, SEQ ID NO: 14, and a homologous sequence thereof having at least 80% (e.g. at least 85%, 90%, 95%, 96%, 97%, 98%, or 99%) sequence identity yet retaining specific binding affinity to CLDN18.2, in particular human CLDN18.2.

In certain embodiments, the anti-CLDN18.2 antibody or an antigen-binding fragment thereof provided herein, comprises a light chain variable region comprising the sequence selected from the group consisting of SEQ ID NO: 15, SEQ ID NO: 16, and a homologous sequence thereof having at least 80% (e.g. at least 85%, 90%, 95%, 96%, 97%, 98%, or 99%) sequence identity yet retaining specific binding affinity to CLDN18.2, in particular human CLDN18.2.

In certain embodiments, the anti-CLDN18.2 antibody comprises a heavy chain variable region and a light chain variable region, wherein
the heavy chain variable region comprises an amino acid sequence selected from the group consisting of SEQ ID NO: 12, SEQ ID NO: 13 and SEQ ID NO: 14, and
the light chain variable region comprises an amino acid sequence selected from the group consisting of SEQ ID NO: 15 and SEQ ID NO: 16.

In certain embodiments, the humanized anti-CLDN18.2 antibodies may comprise the heavy chain variable region fused to the constant region of human IgG1 isotype and the light chain variable region fused to the constant region of human kappa chain.

The humanized anti-CLDN18.2 antibodies provided herein retained the specific binding affinity to a biological sample expressing CLDN18.2, and are at least comparable to, or even better than, the parent antibodies in that aspect. The humanized antibodies provided herein can also retain their functionality in that all antibodies can mediate cell killing by ADCC, CDC and induction of apoptosis induced by cross linking of the target at the tumor cell surface and direct inhibition of proliferation.

In certain embodiments, the anti-CLDN18.2 antibody comprises a heavy chain and a light chain, wherein
the heavy chain comprises an amino acid sequence of SEQ ID NO: 39, and
the light chain comprises an amino acid sequence of SEQ ID NO: 40.

In certain embodiments, the anti-CLDN18.2 antibody is capable of inducing the expression of PD-L1 in the diseased tissue of the subject.

The anti-CLDN18.2 antibodies used in the methods provided herein can also encompass various types of variants of the antibody sequences provided herein.

In certain embodiments, the variants comprise one or more modification(s) or substitution(s) in 1, 2, or 3 CDR sequences as mentioned above, in one or more FR sequences, in the heavy or light chain variable region sequences provided herein, and/or in the constant region (e.g., Fc region). Such antibody variants retain specific binding affinity to CLDN 18.2 of their parent antibodies, but have one or more desirable properties conferred by the modification(s) or substitution(s). For example, the antibody variants may have improved antigen-binding affinity, improved glycosylation pattern, reduced risk of glycosylation, reduced deamination, reduced or increased effector function(s), improved FcRn receptor binding, increased pharmacokinetic half-life, pH sensitivity, and/or compatibility to conjugation (e.g., one or more introduced cysteine residues), to name a few. In certain embodiments, the anti-CLDN18.2 antibodies used in the methods provided herein also encompass a glycosylation variant having improved effector functions such as ADCC or CDC.

In certain embodiments, the anti-CLDN18.2 antibodies used in the methods provided herein is afucosylated. The term “afucosylation,” or “afucosylated,” refers to the reduced or eliminated core-fucose on the N-glycan attached to the antibody. The majority glycans of human IgG antibodies are known as G0, G1 and G2, which are complex biantennary molecules with core fucose residue carrying zero, one or two terminal galactose.

Afucosylated antibody variants can be made using methods known in the art, for example, as described in US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004).

In certain embodiments, the antibody glycosylation variant is afucosylated at Asn297 site of CH2 region in Fc of the antibody. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies.

In certain embodiments, the anti-CLDN18.2 antibodies used in the methods provided herein also encompass a cysteine-engineered variant, which comprises one or more introduced free cysteine amino acid residues. A free cysteine residue is one which is not part of a disulfide bridge. A cysteine-engineered variant is useful for conjugation with, for example a cytotoxic and/or imaging compound, a label, or a radioisoptype among others, at the site of the engineered cysteine, through for example a maleimide or haloacetyl. Methods for engineering antibodies or antigen-binding fragments to introduce free cysteine residues are known in the art, see, for example, WO2006/034488.

In certain embodiments, the constant region of the anti-CLDN 18.2 antibodies or antigen-binding fragments thereof used in the methods provided herein comprises one or more amino acid residue substitutions relative to SEQ ID NO: 9 (i.e. the wild-type sequence), selected from the group consisting of: L235V, F243L, R292P, Y300L, P396L, or any combination thereof. In certain embodiments, the constant region comprises the sequence of SEQ ID NO: 11.

In certain embodiments, the anti-CLDN18.2 antibodies used in the methods provided herein also encompass anti-CLDN18.2 antigen-binding fragments, such as a diabody, a Fab, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a multispecific antibody, a camelized single domain antibody, a nanobody, a domain antibody, or a bivalent domain antibody.

In certain embodiments, the anti-CLDN18.2 antibodies used in the methods provided herein are bivalent, tetravalent, hexavalent, or multivalent. The term “valent” as used herein refers to the presence of a specified number of antigen binding sites in a given molecule. As such, the terms “bivalent”, “tetravalent”, and “hexavalent” denote the presence of two binding site, four binding sites, and six binding sites, respectively, in an antigen-binding molecule. Any molecule being more than bivalent is considered multivalent, encompassing for example, trivalent, tetravalent, hexavalent, and so on.

A bivalent molecule can be monospecific if the two binding sites are both specific for binding to the same antigen or the same epitope. This, in certain embodiments, provides for stronger binding to the antigen or the epitope than a monovalent counterpart. Similar, a multivalent molecule may also be monospecific. In certain embodiments, in a bivalent or multivalent antigen-binding moiety, the first valent of binding site and the second valent of binding site are structurally identical (i.e. having the same sequences), or structurally different (i.e. having different sequences albeit with the same specificity).

A bivalent can also be bispecific, if the two binding sites are specific for different antigens or epitopes. This also applies to a multivalent molecule. For example, a trivalent molecule can be bispecific when two binding sites are monospecific for a first antigen (or epitope) and the third binding site is specific for a second antigen (or epitope). The bi-specific antibodies that can be used in the methods provided herein can target both CLDN 18.2 and a checkpoint molecule, such as PD-1, PD-L1, PD-L2, CLTA-4, SIRPα, TIM-3, LAG3, A2AR, CD160, 2B4, TGFβ, VISTA, BTLA, TIGIT, LAIR1, OX40, CD2, CD27, CD28, CD30, CD40, CD122, ICAM-1, IDO, NKG2C, SLAMF7, SIGLEC7, NKp80, CD160, B7-H3, LFA-1, 1COS, 4-1BB, GITR, BAFFR, HVEM, CD7, LIGHT, IL-2, IL-15, CD3, CD16 or CD83. Exemplary bi-specific antibodies that can be used in the methods provided herein include, without limitation, a bi-specific antibody targeting CLDN 18.2 and CD3, a bi-specific antibody targeting CLDN 18.2 and 4-1BB, a bi-specific antibody targeting CLDN and TGFβ, a bi-specific antibody targeting CLDN 18.2 and SIRPα, and a bi-specific antibody targeting CLDN 18.2 and IL-15.

The anti-CLDAN18.2 antibodies used in the methods provided herein can also be antibody-drug conjugates (ADC) comprising any of the anti-CLDN18.2 antibodies mentioned above conjugated to a cytotoxic agent.

In certain embodiments, the cytotoxic agent can be any agent that is detrimental to cells or that can damage or kill cells. In certain embodiments, the cytotoxic agent is optionally a toxin, a chemotherapeutic agent (such as a DNA-alkylators, a topoisomerase inhibitor, a tubulin-binders, a growth inhibitory agent, or other anticancer drugs), or a radioactive isotope.

Examples of toxins include bacterial toxins and plant toxins, such as for example, diphtheria toxin, exotoxin A chain (from Pseudomonas aeruginosa), ricin, abrin, modeccin, alpha-sarcin, Aleurites fordii. proteins, dianthin proteins, Phytolaca americana proteins (PARI, PAPII, and PAP-S), Momordica charantia inhibitor, curcin, crotin, Sapaonaria officinalis inhibitor, gelonin, restrictocin, phenomycin, enomycin, and the tricothecenes (see, e.g., WO 93/21232). Such a large molecule toxin can be conjugated to the antibodies or antigen-binding fragments provided herein using methods known in the art, for example, as described in Vitetta et al (1987) Science, 238:1098.

The cytotoxic agent can also be small molecule toxins and chemotherapeutic agents, such as geldanamycin (Mandler et al (2000) Jour. of the Nat. Cancer Inst. 92(19):1573-1581; Mandler et al (2002) Bioconjugate Chem. 13:786-791), maytansine and maytansinoids (EP 1391213; Liu et al., (1996) Proc. Natl. Acad. Sci. USA 93:8618-8623; U.S. Pat. No. 5,208,020), calicheam icin (Lode et al (1998) Cancer Res. 58:2928; Hinman et al (1993) Cancer Res. 53:3336-3342), taxol, cytochalasin B, gramicidin D, ethidium bromide, emetine, mitomycin, etoposide, tenoposide, vincristine, vinblastine, vindesine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin and analogs thereof, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine), calicheamicin, maytansinoids, dolastatins, auristatins such as MMAE and MMAF (U.S. Pat. Nos. 5,635,483; 5,780,588), dolostatins, a trichothecene, and CC1065, and the derivatives thereof having cytotoxic activity.

The cytotoxic agent can also be a highly radioactive isotope. Examples include At²¹¹, I¹³¹, I¹²⁵, Y⁹⁰, Re¹⁸⁶, Sm¹⁵³, Bi²¹², P³², Pb²¹² and radioactive isotopes of Lu. Methods of conjugation of a radioisotope to an antibody is known in the art, for example, via a suitable ligand reagent (see, e.g., WO94/11026; Current Protocols in Immunology, Volumes 1 and 2, Coligen et al, Ed. Wiley-Interscience, New York, N.Y, Pubs. (1991)). A ligand reagent has a chelating ligand that can bind, chelate or otherwise complex a radioisotope metal, and also has a functional group that is reactive with a thiol of cysteine of an antibody or antigen-binding fragment. Exemplary chelating ligands include DOTA, DOTP, DOTMA, DTPA and TETA (Macrocyclics, Dallas, Tex.).

In certain embodiments, in the ADC used in the methods provided herein, an antibody (or antigen-binding fragment thereof) is conjugated to one or more cytotoxic agents at an antibody: agent ratio of about 1 to about 20, about 1 to about 6, about 2 to about 6, about 3 to about 6, about 2 to about 5, about 2 to about 4, or about 3 to about 4.

The anti-CLDN18.2 antibodies used in the methods provided herein can also be replaced by immune cells expressing chimeric antigen receptors (CARs) or genetically modified TCRs comprising an anti-CLDN18.2 antigen binding domain as mentioned above and a T-cell activation domain. Chimeric antigen receptors (CARs) are engineered chimeric receptors that combine an antigen-binding domain of an antibody with one or more signaling domains for T cell activation. Immune cells such as T cells and Nature Killer (NK) cells can be genetically engineered to express CARs. T cells expressing a CAR are referred to as CAR-T cells. T cells expressing a genetically modified TCR are referred to as TCR-T cells. CAR and genetically modified TCR can mediate antigen-specific cellular immune activity in the T cells, enabling the CAR-T/TCR-T cells to eliminate cells (e.g. tumor cells) expressing the targeted antigen. In one embodiment, binding of the CAR-T/TCR-T cells provided herein to CLDN18.2 expressed on cells such as cancer cells, results in proliferation and/or activation of said CAR-T/TCR-T cells, wherein said activated CAT-T/TCR-T cells can release cytotoxic factors, e.g. perforin, granzymes, and granulysin, and initiate cytolysis and/or apoptosis of the cancer cells.

In some embodiments, the T-cell activation domain of the CAR comprises a co-stimulatory signaling domain and a TCR signaling domain, which can be linked to each other in a random or in a specified order, optionally with a short peptide linker having a length of, for example, between 2 and 10 amino acids (e.g. glycine-serine doublet linker).

In some embodiment, the CAR further comprises a transmembrane domain. When expressed in cells, the anti-CLDN18.2 antigen binding domain is extracellular, and the T-cell activation domain is intracellular.

In certain embodiments, the CAR comprises an anti-CLDN18.2 antigen binding domain, a transmembrane domain, a costimulatory signaling region, and a TCR signaling domain, wherein the antigen binding domain specifically binds to CLDN18.2 and comprises an antigen-binding fragment of the antibodies provided herein.

In certain embodiments, the CAR or genetically modified TCR is bispecific. In certain embodiments, the bispecific CAR or TCR used in the methods provided herein specifically binds to a first and a second epitope of CLDN18.2, or capable of specifically binding to CLDN18.2 and a second antigen, such as CD3, 4-1BB, TGFβ, SIRPα, and IL-15.

The term “PD-1/PD-L1 axis inhibitor” is a molecule (e.g., small molecules, antibodies, etc.) that inhibits the interaction between PD-1/PD-L1 axis binding partners, such as PD-1 and PD-L1, to remove inhibitory effect of T-cell function (e.g., proliferation, cytokine production, and target cell killing) resulting from signaling on the PD-1/PD-L1 signaling axis. The PD-1/PD-L1 axis inhibitor can include a PD-1 inhibitor or PD-L1 inhibitor.

The term “PD-1 inhibitor”, as used herein, refers to a molecule that decreases, abrogates, inhibits, blocks, or interferes with signal transduction resulting from the interaction of PD-1 with one or more of its binding partners, such as PD-L1, PD-L2. In certain embodiments, PD-1 inhibitor is a molecule that blocks the binding of PD-1 to its binding partners, such as PD-L1, PD-L2. For example, a PD-1 inhibitor can be anti-PD-1 antibodies or antigen binding fragments thereof, fusion proteins, oligopeptides, immunoadhesins and other molecules that decrease, abrogate, inhibit, block, or interfere with signal transduction resulting from the interaction of PD-1 with PD-L1 and/or PD-L2. In some embodiments, the PD-1 inhibitor is an anti-PD-1 antibody. In some embodiments, a PD-1 inhibitor is selected from the group consisting of Nivolumab (OPDIVO; BMS-936558), Dostarlimab (TSR-042), Pembrolizumab (KEYTRUDA; MK-3475), MEDI0680 (AMP-514), MEDI4736, BI 754091, Pidilizumab (CT-011), Cemiplimab (LIBTAYO, REGN2810), Spartalizumab (PDR001), Cetrelimab (JNJ 63723283), Toripalimab (JS001), PF-06801591, Tislelizumab (BGB-A317), AMP-224 (GSK-2661380), ABBV-181, Lambrolizumab or Camrelizuma (SHR-1210), Sintilimab (Tyvyt, IBI308), Penpulimab (AK105).

In some embodiments, the PD-1 inhibitor is Nivolumab (OPDIVO; BMS-936558).

In some embodiments, the PD-1 inhibitor used in the methods provided herein comprises an anti-PD-1 antibody comprises heavy chain HCDR1, HCDR2 and HCDR3 and/or light chain LCDR1, LCDR2 and LCDR3 sequences, wherein:
the HCDR1 sequence comprises NSGMH (SEQ ID NO: 55), or a homologue sequence of at least 80% sequence identity thereof;
the HCDR2 sequence comprises VIWYDGSKRYYADSVKG (SEQ ID NO: 56), or a homologue sequence of at least 80% sequence identity thereof,
the HCDR3 sequence comprises NDDY (SEQ ID NO: 57), or a homologue sequence of at least 80% sequence identity thereof;
the LCDR1 sequence comprises RASQSVSSYLA (SEQ ID NO: 58) or a homologue sequence of at least 80% sequence identity thereof,
the LCDR2 sequence comprises DASNRAT (SEQ ID NO: 59) or a homologue sequence of at least 80% sequence identity thereof;
the LCDR3 sequence comprises QQSSNWPRT (SEQ ID NO: 60) or a homologue sequence of at least 80% sequence identity thereof.

In certain embodiments, the PD-1 inhibitor used in the methods provided herein comprises an anti-PD-1 antibody comprising a heavy chain variable region and a light chain variable region, wherein
the heavy chain variable region comprises an amino acid sequence of SEQ ID NO: 61, and
the light chain variable region comprises an amino acid sequence of SEQ ID NO: 62.

In certain embodiments, the PD-1 inhibitor used in the methods provided herein comprises an anti-PD-1 antibody comprising a heavy chain and a light chain, wherein
the heavy chain comprises an amino acid sequence of SEQ ID NO: 63, and
the light chain comprises an amino acid sequence of SEQ ID NO: 64.

The term “PD-L1 inhibitor” is a molecule that decrease, abrogate, inhibit, block, or interfere with signal transduction resulting from the interaction of PD-L1 with one or more of its binding partners, such as PD-1, B7-1. For example, the PD-L1 inhibitor can be anti-PD-L1 antibodies or antigen binding fragments thereof, fusion proteins, immunoadhesins, oligopeptides and other molecules that decrease, abrogate, inhibit, block, or interfere with signal transduction resulting from the interaction of PD-L1 with one or more of its binding partners, such as PD-1, B7-1. In some embodiments, a PD-L1 inhibitor is an anti-PD-L1 antibody. In some embodiments, an anti-PD-L1 antibody is selected from the group consisting of Atezolizumab (TECENTRIQ; R05541267; MPDL3280A; RG7446), BMS-936559, Avelumab (bavencio), lodapolimab (LY3300054), Durvalumab (MEDI4736), CX-072 (Proclaim-CX-072), FAZ053, KN035, MDX-1105, STI-1040, CS1001, Adebrelimab (SHR-1316), and SHR-1701.

In some embodiments, the PD-L1 inhibitor used in the methods provided herein is a bispecific antibody targeting both PD-L1 and another checkpoint molecule, such as, PD-1, PD-L1, PD-L2, CLTA-4, SIRPα, TIM-3, LAG3, A2AR, CD160, 2B4, TGFβ, VISTA, BTLA, TIGIT, LAIR1, OX40, CD2, CD27, CD28, CD30, CD40, CD122, ICAM-1, IDO, NKG2C, SLAMF7, SIGLEC7, NKp80, CD160, B7-H3, LFA-1, 1COS, 4-1BB, GITR, BAFFR, HVEM, CD7, LIGHT, IL-2, IL-15, CD3, CD16 or CD83. In certain embodiments, the PD-L1 inhibitor used in the methods provided herein is a bispecific antibody targeting both PD-L1 and another checkpoint molecule, such as TGFβ, 4-1BB, CTLA4, LAG3 or TIGIT.

In certain embodiments, the PD-L1 inhibitor used in the methods provided herein comprises an anti-PD-L1 antibody comprising heavy chain HCDR1, HCDR2 and HCDR3 and/or light chain LCDR1, LCDR2 and LCDR3 sequences, wherein:
the HCDR1 sequence comprises DYYMN (SEQ ID NO: 22), or a homologue sequence of at least 80% sequence identity thereof;
the HCDR2 sequence comprises DINPNNAETLYNHKFKG (SEQ ID NO: 23), or a homologue sequence of at least 80% sequence identity thereof,
the HCDR3 sequence comprises WGDGPFAY (SEQ ID NO: 24), or a homologue sequence of at least 80% sequence identity thereof,
the LCDR1 sequence comprises KASQNVGAAVA (SEQ ID NO: 25) or a homologue sequence of at least 80% sequence identity thereof,
the LCDR2 sequence comprises SVSDRYT (SEQ ID NO: 26) or a homologue sequence of at least 80% sequence identity thereof;
the LCDR3 sequence comprises QQYSNYPT (SEQ ID NO: 27) or a homologue sequence of at least 80% sequence identity thereof.

In certain embodiments, the PD-L1 inhibitor used in the methods provided herein comprises an anti-PD-L1 antibody comprising a heavy chain variable region and a light chain variable region, wherein
the heavy chain variable region comprises an amino acid sequence of SEQ ID NO: 17, and
the light chain variable region comprises an amino acid sequence of SEQ ID NO: 18.

In certain embodiments, the PD-L1 inhibitor used in the methods provided herein comprises an anti-PD-L1 antibody comprising a heavy chain and a light chain, wherein
the heavy chain comprises an amino acid sequence of SEQ ID NO: 19 or SEQ ID NO: 20, and
the light chain comprises an amino acid sequence of SEQ ID NO: 21.

In certain embodiments, the PD-L1 inhibitor used in the methods provided herein comprises an anti-PD-L1 antibody comprising heavy chain HCDR1, HCDR2 and HCDR3 and/or light chain LCDR1, LCDR2 and LCDR3 sequences, wherein:
the HCDR1 sequence comprises TYWMH (SEQ ID NO: 32), or a homologue sequence of at least 80% sequence identity thereof;
the HCDR2 sequence comprises MIQPNSGGTKYNEKFKK (SEQ ID NO: 33), or a homologue sequence of at least 80% sequence identity thereof,
the HCDR3 sequence comprises GAGTVDYFDY (SEQ ID NO: 34), or a homologue sequence of at least 80% sequence identity thereof,
the LCDR1 sequence comprises RASESVDIYGNSFMH (SEQ ID NO: 35) or a homologue sequence of at least 80% sequence identity thereof;
the LCDR2 sequence comprises RASNLES (SEQ ID NO: 36) or a homologue sequence of at least 80% sequence identity thereof;
the LCDR3 sequence comprises QQSTEDPYT (SEQ ID NO: 37) or a homologue sequence of at least 80% sequence identity thereof.

In certain embodiments, the PD-L1 inhibitor used in the methods provided herein comprises an anti-PD-L1 antibody comprising a heavy chain variable region and a light chain variable region, wherein
the heavy chain variable region comprises an amino acid sequence of SEQ ID NO: 28, and
the light chain variable region comprises an amino acid sequence of SEQ ID NO: 29.

In certain embodiments, the PD-L1 inhibitor used in the methods provided herein comprises an anti-PD-L1 antibody comprising a heavy chain and a light chain, wherein
the heavy chain comprises an amino acid sequence of SEQ ID NO: 30, and
the light chain comprises an amino acid sequence of SEQ ID NO: 31.

In certain embodiments, the PD-L1 inhibitor used in the methods provided herein comprises an anti-PD-L1 antibody comprising heavy chain HCDR1, HCDR2 and HCDR3 and/or light chain LCDR1, LCDR2 and LCDR3 sequences, wherein:
the HCDR1 sequence comprises DSWIH (SEQ ID NO: 45), or a homologue sequence of at least 80% sequence identity thereof;
the HCDR2 sequence comprises WISPYGGSTYYADSVKG (SEQ ID NO: 46), or a homologue sequence of at least 80% sequence identity thereof,
the HCDR3 sequence comprises RHWPGGFDY (SEQ ID NO: 47), or a homologue sequence of at least 80% sequence identity thereof,
the LCDR1 sequence comprises RASQDVSTAVA (SEQ ID NO: 48) or a homologue sequence of at least 80% sequence identity thereof,
the LCDR2 sequence comprises SASFLYS (SEQ ID NO: 49) or a homologue sequence of at least 80% sequence identity thereof;
the LCDR3 sequence comprises QQYLYHPAT (SEQ ID NO: 50) or a homologue sequence of at least 80% sequence identity thereof.

In certain embodiments, the PD-L1 inhibitor used in the methods provided herein comprises an anti-PD-L1 antibody comprising a heavy chain variable region and a light chain variable region, wherein
the heavy chain variable region comprises an amino acid sequence of SEQ ID NO: 51, and
the light chain variable region comprises an amino acid sequence of SEQ ID NO: 52.

In certain embodiments, the PD-L1 inhibitor used in the methods provided herein comprises an anti-PD-L1 antibody comprising a heavy chain and a light chain, wherein
the heavy chain comprises an amino acid sequence of SEQ ID NO: 53, and
the light chain comprises an amino acid sequence of SEQ ID NO: 54.

The CLDN18.2 antagonist and PD-1/PD-L1 axis inhibitor used in the methods provided herein may be administered by any route known in the art, such as for example parenteral (e.g., subcutaneous, intraperitoneal, intravenous, including intravenous infusion, intramuscular, or intradermal injection) or non-parenteral (e.g., oral, intranasal, intraocular, sublingual, rectal, or topical) routes. In certain embodiments, the administration is via oral, nasal, intravenous, subcutaneous, sublingual, or intramuscular administration.

In certain embodiments, the administration of the composition comprising anti-CLDN18.2 antibody is prior to, simultaneously with, or after the administration of the composition comprising PD-1/PD-L1 axis inhibitor.

In certain of these embodiments, a CLDN18.2 antagonist that is administered in combination with one or more PD-1/PD-L1 axis inhibitors in the methods provided herein may be administered simultaneously with the one or more PD-1/PD-L1 axis inhibitors, and in certain of these embodiments the CLDN18.2 antagonist and the one or more PD-1/PD-L1 axis inhibitors may be administered as part of the same pharmaceutical composition. However, a CLDN18.2 antagonist administered “in combination” with a PD-1/PD-L1 axis inhibitor does not have to be administered simultaneously with or in the same composition as the agent. A CLDN18.2 antagonist administered prior to or after a PD-1/PD-L1 axis inhibitor is considered to be administered “in combination” with the PD-1/PD-L1 axis inhibitor as the phrase is used herein, even if the CLDN18.2 antagonist and the PD-1/PD-L1 axis inhibitor are administered via different routes. Where possible, the PD-1/PD-L1 axis inhibitor administered in combination with the CLDN18.2 antagonist are administered according to the schedule listed in the product information sheet of the PD-1/PD-L1 axis inhibitor, or according to the Physicians' Desk Reference 2003 (Physicians' Desk Reference, 57th Ed; Medical Economics Company; ISBN: 1563634457; 57th edition (November 2002)) or protocols well known in the art.

In another aspect, the present disclosure provides use of a pharmaceutical composition, comprising a therapeutically effective amount of a) a CLDN18.2 antagonist, b) a PD-1/PD-L1 axis inhibitor, or c) both, and one or more pharmaceutically acceptable carriers, in the manufacture of a medicament for treating a disease or condition in a subject in need thereof. In some embodiments, the disease or condition is characterized in having: a) CLDN18.2 expression in a diseased tissue, and b) low or no expression of PD-L1 in the diseased tissue.

### II. Kit

In another aspect, the present disclosure provides kits useful in treating a disease or condition in a subject in need thereof, comprising a first container that comprises a CLDN18.2 antagonist and a second container that comprises a PD-1/PD-L1 axis inhibitor, and optionally instructions for use of the kit. In some embodiments, the disease or condition is characterized in having: a) CLDN18.2 expression in a diseased tissue, and b) low or no expression of PD-L1 in the diseased tissue. Suitable containers include, for example, vials (e.g., dual chamber vials), bottles, syringes (such as single or dual chamber syringes) and test tubes. The container may be formed from various materials such as glass or plastic.

In another aspect, the present disclosure provides kits comprising a CLDN18.2 antagonist and a package insert comprising instructions for using the CLDN18.2 antagonist in combination with a PD-1/PD-L1 axis inhibitor to treat a disease or condition in a subject in need thereof. In some embodiments, the disease or condition is characterized in having: a) CLDN18.2 expression in a diseased tissue, and b) low or no expression of PD-L1 in the diseased tissue.

In another aspect, the present disclosure provides kits comprising a PD-1/PD-L1 axis inhibitor and a package insert comprising instructions for using the PD-1/PD-L1 axis inhibitor in combination with a CLDN18.2 antagonist to treat a disease or condition in a subject in need thereof. In some embodiments, the disease or condition is characterized in having: a) CLDN18.2 expression in a diseased tissue, and b) low or no expression of PD-L1 in the diseased tissue.

In another aspect, the present disclosure provides a kit comprising an anti-CLDN18.2 antibody or antigen-binding fragment thereof and a package insert comprising instructions for using the anti-CLDN18.2 antibody or antigen-binding fragment thereof in combination with a PD-1/PD-L1 axis inhibitor to treat a disease or condition in a subject having low expression of CLDN18.2 and/or PD-L1. Any of the PD-1/PD-L1 axis inhibitor described herein or to be shown effective in inhibiting the PD-1/PD-L1 axis signaling may be included in the kit.

As used herein, the term “package insert” refers to instructions included in a commercial package of medicines that contain information about, for example, indications, dosage, usage, administration, contraindications, other medicines to be combined with the packaged product, and/or warnings concerning the use of such medicines.

In another aspect, the present disclosure also provides a kit for predicting responsiveness of a subject to treatment with a CLDN 18.2 antagonist in combination with PD-1/PD-L1 axis inhibitor, comprising: one or more reagents for detecting presence of CLDN 18.2 and/or PD-L1 in a biological sample obtained from the subject; or one or more reagents for measuring expression level of CLDN 18.2 and/or PD-L1 in a biological sample obtained from the subject. In certain embodiments, the biological sample is a tumor tissue. In certain embodiments, the kit further comprises instructions on predicting responsiveness of a subject to the treatment with a CLDN 18.2 antagonist in combination with PD-1/PD-L1 axis inhibitor. The instruction may comprise the contents as described above.

The kit may further comprise other materials desirable from a commercial and user standpoint, including other buffers, diluents, filters, needles, and syringes.

### III. Disease or Conditions

The disease or condition to be diagnosed, sensitized, or treated by the methods or kits provided herein can be cancer. In certain embodiments, the cancer is selected from the group consisting of gastric cancer, lung cancer, bronchial cancer, bone cancer, liver and bile duct cancer, pancreatic cancer, breast cancer, liver cancer, ovarian cancer, testicle cancer, kidney cancer, bladder cancer, head and neck cancer, spine cancer, brain cancer, cervix cancer, uterine cancer, endometrial cancer, colon cancer, colorectal cancer, rectal cancer, anal cancer, esophageal cancer, gastrointestinal cancer, skin cancer, prostate cancer, pituitary cancer, stomach cancer, vagina cancer, thyroid cancer, glioblastoma, astrocytoma, melanoma, myelodysplastic syndrome, sarcoma, teratoma, and adenocarcinoma.

Examples of cancers include but are not limited to, non-small cell lung cancer (squamous/nonsquamous), small cell lung cancer, renal cell cancer, colorectal cancer, colon cancer, ovarian cancer, breast cancer (including basal breast carcinoma, ductal carcinoma and lobular breast carcinoma), pancreatic cancer, gastric carcinoma, bladder cancer, esophageal cancer, mesothelioma, melanoma, head and neck cancer, thyroid cancer, sarcoma, prostate cancer, glioblastoma, cervical cancer, thymic carcinoma, melanoma, myelomas, mycoses fungoids, merkel cell cancer, hepatocellular carcinoma (HCC), fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, lymphoid malignancy, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, hepatoma, bile duct carcinoma, choriocarcinoma, Wilms' tumor, cervical cancer, testicular tumor, seminoma, classical Hodgkin lymphoma (CHL), primary mediastinal large B-cell lymphoma, T-cell/histiocyte-rich B-cell lymphoma, acute lymphocytic leukemia, acute myelocytic leukemia, acute myelogenous leukemia, chronic myelocytic (granulocytic) leukemia, chronic myelogenous leukemia, chronic lymphocytic leukemia, polycythemia vera, mast cell derived tumors, EBV-positive and -negative PTLD, and diffuse large B-cell lymphoma (DLBCL), plasmablastic lymphoma, extranodal NK/T-cell lymphoma, nasopharyngeal carcinoma, HHV8-associated primary effusion lymphoma, non-Hodgkin's lymphoma, multiple myeloma, Waldenstrom's macroglobulinemia, heavy chain disease, myelodysplastic syndrome, hairy cell leukemia and myelodysplasia, primary CNS lymphoma, spinal axis tumor, brain stem glioma, astrocytoma, medulloblastoma, craniopharyogioma, ependymoma, pinealoma, hemangioblastoma, acoustic neuroma, oligodendroglioma, menangioma, melanoma, neuroblastoma and retinoblastoma.

In certain embodiments, the cancer is gastric cancer, lung cancer, colon cancer, or combination thereof.

In certain embodiments, the cancer is a CLDN18.2-expressing cancer. “CLDN18.2-expressing cancer” as used herein refers to any cancer or tumor involving cancer cells expressing CLDN18.2.

Examples of CLDN18.2-expressing cancer include, without limitation, gastric cancer, esophageal cancer, pancreatic cancer, lung cancer such as non-small cell lung cancer (NSCLC) and small cell lung cancer (SCLC), ovarian cancer, colon cancer, colorectal cancer, gastrointestinal stromal tumors (GIST), gastrointestinal carcinoid tumors, rectal cancer, anal cancer, bile duct cancer, small intestine cancer, appendix cancer; prostate cancer, renal cancer (e.g., renal cell carcinoma), hepatic cancer, head-neck cancer, and cancer of the gallbladder and metastases thereof, for example, gastric cancer metastasis such as Krukenberg tumors, peritoneal metastasis and lymph node metastasis.

In certain embodiments, the CLDN18.2-expressing cancer can be an adenocarcinoma, for example, an advanced adenocarcinoma. In certain embodiments, the cancer is selected from adenocarcinomas of the stomach, the esophagus, the pancreatic duct, the bile ducts, the lung and the ovary. In certain embodiments, the CLDN18.2-expressing cancer comprises a cancer of the stomach, a cancer of the esophagus, in particular the lower esophagus, a cancer of the eso-gastric junction and gastroesophageal cancer.

### EXAMPLES

While the disclosure has been particularly shown and described with reference to specific embodiments (some of which are preferred embodiments), it should be understood by those having skill in the art that various changes in form and detail may be made therein without departing from the spirit and scope of the present disclosure as disclosed herein.

### Example 1: Upregulation of PD-L1 Expression on Tumor Cells by Anti-Claudin18.2 (CLDN18.2) Antibody in the Presence of PBMC

In this assay, three different CLDN18.2-expressing gastric tumor cell lines, NUGC-4 (JCRB, Cat #JCRBB0834), KATOIII (ATCC, Cat #HTB-103) and SNU-620 (Cobioer, Cat #CBP60508), were used as target cell and added to the cell culture plate. No antibody, 30 μg/ml of anti-CLDN18.2 antibody 18B10, 30 μg/ml of isotype control hIgG1 or 5 ng/ml IFN-γ were added into the corresponding wells with the target cell. After the plate was co-cultured at 37° C. for 30 mins, human PBMC as the effector cell was added with a E/T ratio of 40:1. The plate was incubated at 37° C. for 72 hours. At the end of incubation, 100 μl of cell culture supernatant of each well was transferred to another cell culture plate with the same target cell planted. After the second plate was incubated at 37° C. for 72 hours, the target cells were collected and analyzed for PD-L1 expression by flowcytometry using mouse anti-human PD-L1 antibody (BD Pharmingen, Cat #557924). S1 stood for the supernatant of the target cell alone; S2 stood for the supernatant of the target cell and human PBMC with isotype control hIgG1; S3 stood for the supernatant of the target cell and human PBMC with anti-CLDN18.2 antibody 18B10; S4 stood for the supernatant of the target cell with IFN-γ.

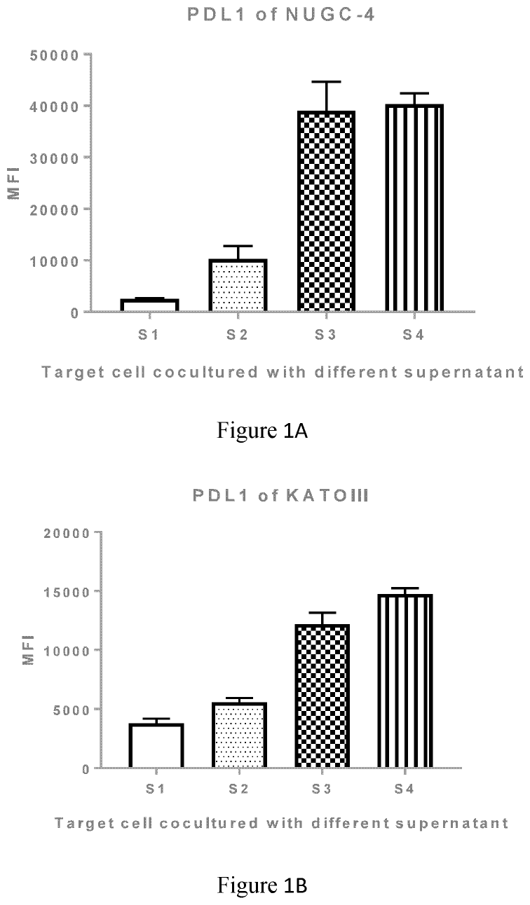

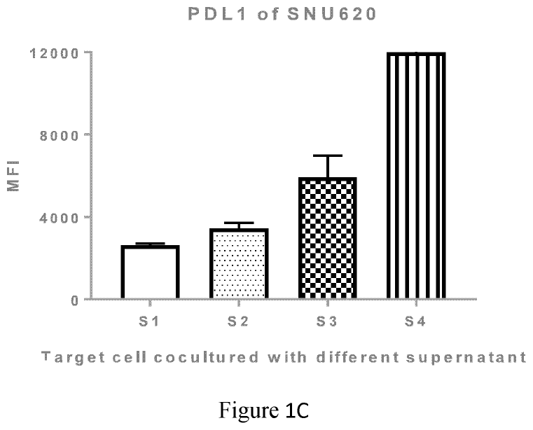

As shown in FIGS. 1A-1C, when compared to S1 group that expressed a base level of PD-L1 in the target cell, S3 and S4 groups showed increased PD-L1 expression while S2 group had only a marginal change. This result indicated that anti-CLDN18.2 antibody could upregulate PD-L1 expression of the target tumor cells. This raises the possibility of potential synergy when both anti-CLDN 18.2 antibody and PD-1/PD-L1 axis inhibitors are combined for the cancer treatment.

### Example 2: Combination of Anti-CLDN18.2 Antibody and Anti-PD-L1 Antibody AM4B6 in Tumor Models

To assess the potential synergy of combining claudin 18.2 antibody and checkpoint inhibitor such as anti-PDL1 antibody, the following experiments were conducted using both NCI-H460-CLDN18.2 xenograft model (as lung cancer model), CT-26-CLDN18.2 syngeneic model (as colorectal cancer model) and MC38-CLDN18.2 syngeneic model (as colon adenocarcinoma model).

### 1. The Anti-Tumor Efficacy of Anti-CLDN18.2 Antibody and Anti-PD-L1 Antibody in NCI-H460-CLDN18.2 Xenograft Tumor Model

NCI-H460-CLDN18.2 was purchased from Kyinno (Cat #, KC-1450), which was stablely transfected with human CLDN18.2 expression. The tumor cells were routinely sub-cultured twice weekly by trypsin-EDTA treatment (Hyclone). The cells growing in an exponential growth phase were harvested and counted for tumor inoculation. Female SPF grade NOD-SCID mice were inoculated with 3×10\hat{ }6 NCI-H460-CLDN18.2 cells and 1.5×10\hat{ }6 human PBMC along with 50% matrigel. Animal were selected and randomized to 4 groups (n=10), approximately 4 hours post inoculation. Animals were then treated with 10 mg/kg Isotype control, 10 mg/kg anti-CLDN18.2 antibody 18B10, 10 mg/kg anti-PD-L1 antibody AM4B6, 10 mg/kg anti-CLDN18.2 antibody 18B10 and 10 mg/kg anti-PD-L1 antibody AM4B6 twice a week for 5 weeks by i.p. injection. Tumor size was measured twice or triple times a week in two dimensions using a caliper (INSIZE) and the volume was expressed in mm\hat{ }3 using the formula: V=0.5 a×b\hat{ }2 where a and b are the long and short diameters of the tumor, respectively. Results were analyzed using Prism GraphPad and expressed as mean±S.E.M. Comparisons between two groups were made by T-test, and the difference is considered significant if p is *<0.05 and **<0.01.

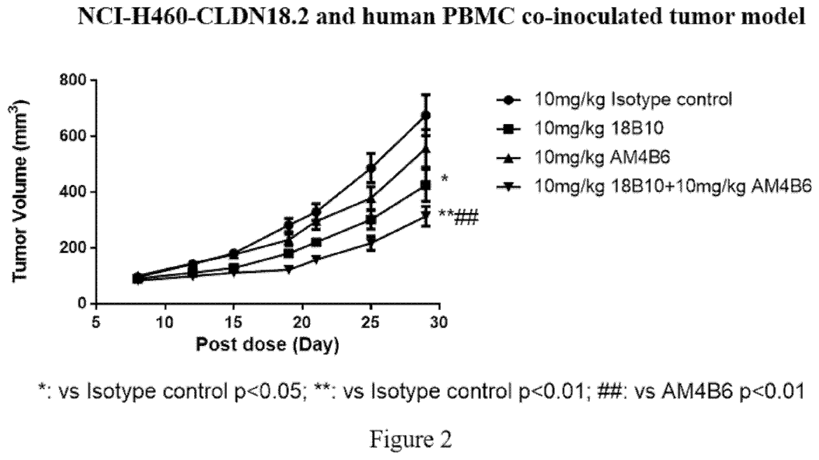

As shown in FIG. 2, compared with the isotype control, anti-CLDN18.2 antibody 18B10 could slightly (yet statistically significant) inhibit tumor growth, whereas anti-PD-L1 antibody AM4B6 alone had no significant impact on tumor growth. Specifically, the AM4B6 group only had very slight inhibition with TGI of 17.6% (Table 1). In contrast, combination of the two antibodies had a significant improved effect on tumor growth and the TGI was increased to 53.6% (Table 1).

TABLE 1
Tumor Growth Inhibition of combination therapy
on NCI-H460-CLDN18.2 and hPBMC Co-inoculation
xenograft tumor model on Day 29

| Treatment | Tumor size | TGI | P value vs |
|---|---|---|---|
| (n = 10) | mm³ | (%) | Isotype control |
| Isotype control 10 mg/kg | 675.86 ± 73.50 | / | / |
| 18B10 10 mg/kg | 425.01 ± 58.32 | 37.1 | 0.0162 |
| AM4B6 10 mg/kg | 557.06 ± 67.47 | 17.6 | 0.2514 |
| 18B10 + AM4B6 10 + 10 mg/kg | 313.57 ± 35.36 | 53.6 | 0.0004 |

### 2. The Anti-Tumor Efficacy of Anti-CLDN18.2 Antibody and Anti-PD-L1 Antibody in CT26-CLDN18.2 Syngeneic Tumor Model

CT26 is synergic mouse tumor model. CT26-CLDN18.2 was purchased from Kyinno (Cat #, KC-1195), which was stalely transfected with human CLDN18.2 expression. The tumor cells were routinely sub-cultured twice weekly by trypsin-EDTA treatment (Hyclone). The cells growing in an exponential growth phase were harvested and counted for tumor inoculation. Female SPF grade BALB/c mice were inoculated with 2×10\hat{ }6 CT26-CLDN18.2 cells. Animal were selected and randomized to 4 groups (n=8) when tumors grow to 80-120 mm³. Animals were then treated with either 10 mg/kg Isotype control, 10 mg/kg anti-CLDN18.2 antibody 18B10, 3 mg/kg anti-PD-L1 antibody Atezolizumab, or 10 mg/kg anti-CLDN18.2 antibody 18B10 and 3 mg/kg anti-PD-L1 antibody Atezolizumab twice a week for 3 weeks by i.p. injection. Tumor size was measured twice a week in two dimensions using a caliper (INSIZE) and the volume was expressed in mm\hat{ }3 using the formula: V=0.5 a×b\hat{ }2 where a and b are the long and short diameters of the tumor, respectively. Results were analyzed using Prism GraphPad and expressed as mean±S.E.M. Comparisons between two groups were made by T-test, and the difference is considered significant if p is *<0.05 and **<0.01.

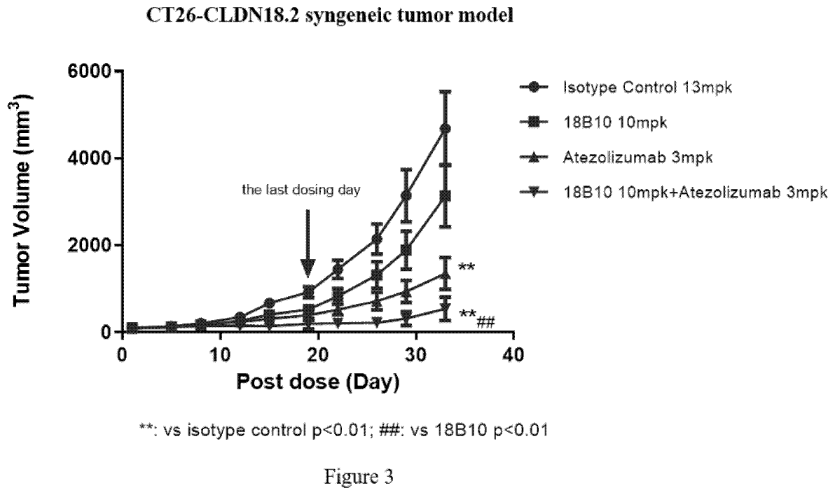

As shown in FIG. 3, after administration, the tumor volume of mice in 10 mg/kg isotype control group continued to increase. The treatment with 10 mg/kg 18B10, 3 mg/kg Atezolizumab, and combination of 10 mg/kg 18B10 and 3 mg/kg Atezolizumab all showed better antitumor activity compared with treatment with isotype control, shown in FIG. 3. Furthermore, the tumor inhibition activity of the combination group lasted for about 2 weeks after the last dosing day (Day 19), indicating there may be some immune memory effect of the combination treatment. And the TGI of combination of the two antibodies was increased to 88.44% (Table 2).

TABLE 2
Tumor Growth Inhibition of combination therapy on
CT26-CLDN18.2 syngeneic tumor model on Day 33

| | | | | P value vs |
|---|---|---|---|---|
| Treatment | Tumor size | TGI | | Isotype |
| (n = 10) | mm³ | (%) | CR | control |
| Isotype control 10 mg/kg | 4681.63 ± 847.75 | / | 0/8 | / |
| 18B10 10 mg/kg | 3130.96 ± 714.01 | 33.12 | 0/8 | >0.05 |
| Atezolizumab 3 mg/kg | 1350.18 ± 366.69 | 71.16 | 2/8 | <0.01 |
| 18B10 + Atezolizumab | 541.23 ± 269.30 | 88.44 | 5/8 | <0.01 |
| 10 + 3 mg/kg | | | | |

### 3. The Anti-Tumor Efficacy of Anti-CLDN18.2 Antibody and Anti-PD-L1 Antibody in MC38-CLDN18.2 Syngeneic Tumor Model

MC38-CLDN18.2 was purchase from Kyinno (Cat #, KC-1449), which was stable transfected with human CLDN18.2 expression. The tumor cells were routinely sub-culture twice weekly by trypsin-EDTA treatment (Hyclone). The cells growing in an exponential growth phase were harvested and counted for tumor inoculation. Female SPF grade C57BL/6 mice were inoculated with 2×10\hat{ }6 MC38-CLDN18.2 cells. Animal were selected and randomized to 4 groups (n=6) when tumors grow to 70-100 mm³. Animals were then treated with 13 mg/kg Isotype control, 10 mg/kg anti-CLDN18.2 antibody 18B10, 3 mg/kg anti-PD-L1 antibody Atezolizumab, 10 mg/kg anti-CLDN18.2 antibody 18B10 and 3 mg/kg anti-PD-L1 antibody Atezolizumab twice a week for 4 weeks by i.p. injection. Tumor size was measured twice a week in two dimensions using a caliper (INSIZE) and the volume was expressed in mm\hat{ }3 using the formula: V=0.5 a×b\hat{ }2 where a and b are the long and short diameters of the tumor, respectively. Results were analyzed using Prism GraphPad and expressed as mean±S.E.M. Comparisons between two groups were made by T-test, and the difference is considered significant if p is *<0.05 and **<0.01.

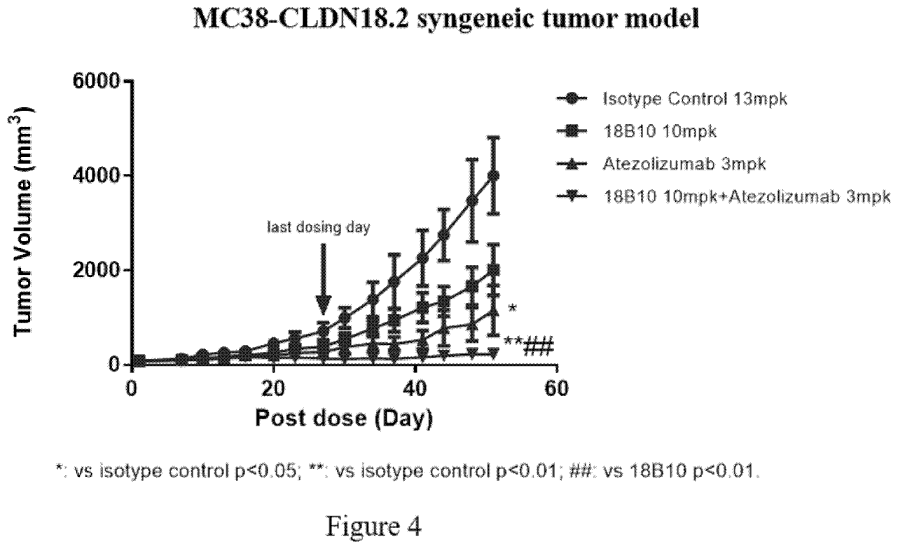

As shown in FIG. 4, after administration, the treatment with either 10 mg/kg 18B10, 3 mg/kg Atezolizumab, or combination of 10 mg/kg 18B10 and 3 mg/kg Atezolizumab all showed better antitumor activity compared with treatment with isotype control, shown in FIG. 4. Furthermore, the tumor inhibition activity of the combination group lasted for about 3 weeks after the last dosing day (Day 28), which is similar to the efficacy data of CT26-CLDN18.2 model. The combination of 18B10 with the PD-L1 Ab may have added some immune memory effect to keep the anti-tumor activity for a long time. And the TGI of combination of the two antibodies was increased to 94.34% (Table 3).

TABLE 3
Tumor Growth Inhibition of combination therapy on
MC38-CLDN18.2 syngeneic tumor model on Day 51

| | | | | P value vs |
|---|---|---|---|---|
| Treatment | Tumor size | TGI | | Isotype |
| (n = 10) | mm³ | (%) | CR | control |
| Isotype control 13 mg/kg | 4000.89 ± 806.98 | / | 0/6 | / |
| 18B10 10 mg/kg | 2008.68 ± 536.32 | 49.79 | 0/6 | >0.05 |
| Atezolizumab 3 mg/kg | 1154.61 ± 526.35 | 71.14 | 0/6 | <0.05 |
| 18B10 + Atezolizumab | 226.42 ± 99.82 | 94.34 | 1/6 | <0.01 |
| 10 + 3 mg/kg | | | | |

### Example 3: Combination of Anti-CLDN18.2 Antibody and Anti-PD-1 Antibody RMP1-14 in Tumor Models

The anti-tumor efficacy of anti-CLDN18.2 antibody and anti-PD-1 antibody was also evaluated in CT26-CLDN18.2 syngeneic tumor model

CT26-CLDN18.2 cells were routinely sub-culture twice weekly by trypsin-EDTA treatment (Hyclone). The cells growing in an exponential growth phase were harvested and counted for tumor inoculation. Female SPF grade BALB/c mice were inoculated with 2×10\hat{ }6 CT26-CLDN18.2 cells. Animal were selected and randomized to 4 groups (n=10) when tumors grow to around 80 mm³. Animals were then treated with 10 mg/kg hIgG1 control and 5 mg/kg 2A3 (rat IgG2a control), 10 mg/kg anti-CLDN18.2 antibody 18B10 and 5 mg/kg 2A3, 5 mg/kg rat anti-mouse PD-1 antibody RMP1-14 (Purchased from BioXcell, US) and 10 mg/kg hIgG1 control, and 10 mg/kg anti-CLDN18.2 antibody 18B10 combined with 5 mg/kg RMP1-14 twice a week for 4 weeks by i.p. injection. Tumor size was measured twice a week in two dimensions using a caliper (INSIZE) and the volume was expressed in mm\hat{ }3 using the formula: V=0.5 a×b\hat{ }2 where a and b are the long and short diameters of the tumor, respectively. Results were analyzed using Prism GraphPad and expressed as mean±S.E.M. Comparisons between two groups were made by T-test, and the difference is considered significant if p is *<0.05 and **<0.01.

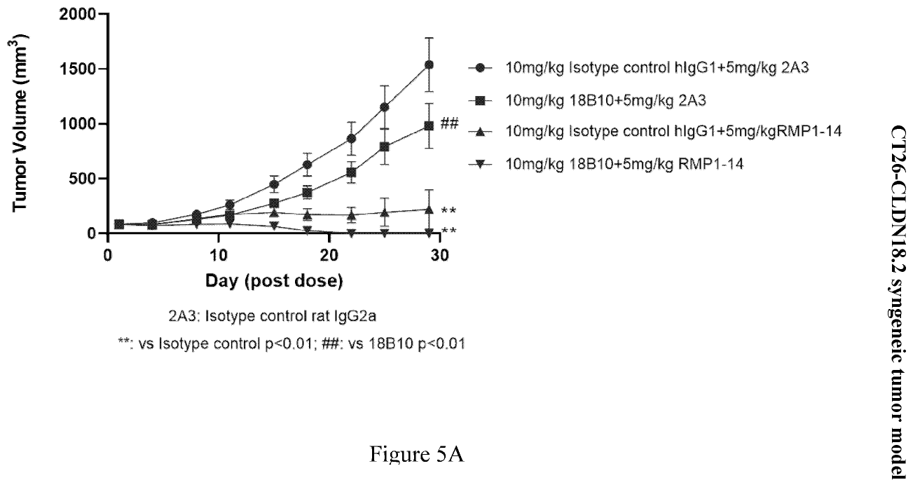

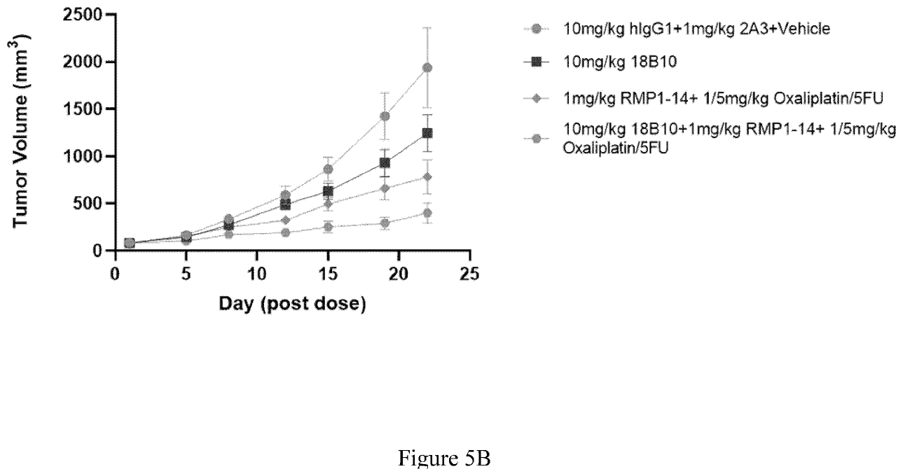

As shown in FIG. 5A, after administration, the tumor volume of mice in 10 mg/kg isotype control group continued to increase. Treatment with either 10 mg/kg 18B10+5 mg/kg 2A3, 10 mg/kg isotype control hIgG1+5 mg/kg RMP1-14, or combination of 10 mg/kg 18B10 and 5 mg/kg RMP1-14 groups all showed better antitumor activity compared with treatment with isotype control group, shown in FIG. 5A. Furthermore, the tumors of the combination group (18B10+RMP1-14) all regressed (TGI=100%) on Day 29, while there was no tumor regression in 18B10+2A3 group and only half of tumors regressed in isotype control+RMP1-14 group (Table 4).

TABLE 4
Tumor Growth Inhibition of combination therapy on
CT26-CLDN18.2 syngeneic tumor model on Day 29

| | | | | P value vs |
|---|---|---|---|---|
| Treatment | Tumor size | TGI | | Isotype |
| (n = 10) | mm³ | (%) | CR | control |
| 10 mg/kg Isotype control | 1536.9 ± 243.37 | / | 0/10 | / |
| hIgG1 + 5 mg/kg 2A3 | | | | |
| 10 mg/kg 18B10 + 5 | 980.36 ± 203.07 | 36.21 | 0/10 | >0.05 |
| mg/kg 2A3 | | | | |
| 10 mg/kg Isotype control | 220.88 ± 176.85 | 85.63 | 5/10 | <0.01 |
| hIgG1 + 5 mg/kg RMP1- | | | | |
| 14 | | | | |
| 10 mg/kg 18B10 + 5 | 0 ± 0 | 100.00 | 10/10 | <0.01 |
| mg/kg RMP1-14 | | | | |

### Example 4: Efficacy of 18B10 Combination with Anti-PD-1 and Chemo on CT26-hCLDN18.2 Tumor Model

Mouse colon cancer cell line CT26 was transfected with CLDN18.2 gene screened stable expressing CLDN18.2 is named CT26-hCLDN18.2. CT26-hCLDN18.2 cells were maintained in vitro as a monolayer culture in RPMI1640 medium (Thermo Fisher) supplemented with 10% heat inactivated fetal bovine serum (ExCell Biology), 100 U/ml penicillin, 100 ug/ml streptomycin (Hyclone) and 1 ug/mL puromycin (Gibco) at 37° C. in an atmosphere with 5% CO₂ in air. The tumor cells were routinely sub-culture twice weekly by trypsin-EDTA treatment (Hyclone). The cells growing in an exponential growth phase were harvested and counted for tumor inoculation. Female SPF grade BABL/c mice were inoculated with mixed 2*10\hat{ }6 CT26-hCLDN18.2 cells with 50% matri-gel. When the tumor size around 80 mm\hat{ }3, tumor bearing mice were selected and randomized to 4 groups (n=10). Animals were treated with 10 mg/kg hIgG1 control and 1 mg/kg 2A3 (anti-rat IgG2a control), 10 mg/kg 18B10, 1 mg/kg RMP1-14 (anti-mouse PD1) and 1 mg/kg Oxaliplatin and 5 mg/kg 5FU, and 10 mg/kg 18B10 combined with 1 mg/kg RMP1-14 (anti-mouse PD1) and 1 mg/kg Oxaliplatin and 5 mg/kg 5FU, twice a week for 3 weeks by i.p. injection for antibody, once weekly for 3 weeks by i.v. injection for chemo. Tumor size was measured twice or triple times a week in two dimensions using a caliper (INSIZE) and the volume was expressed in mm\hat{ }3 using the formula: V=0.5 a*b\hat{ }2 where a and b are the long and short diameters of the tumor, respectively. Results were analyzed using Prism GraphPad and expressed as mean±S.E.M. Comparisons between two groups were made by T-test, and the difference was considered significant if p was *<0.05 and **<0.01. Three-agent combination therapy had enhanced antitumor activity than 18B10 alone and anti-PD-1 plus chemo combination, as shown in Table 5 and FIG. 5B.

TABLE 5
Efficacy of 18B10 combination with anti-PD-1 and
Chemo on CT26-hCLDN18.2 tumor model on Day 22

| | Tumor size | | p value vs | p value vs |
|---|---|---|---|---|
| Treatment | (mm\hat{ }3, | TGI | Isotype | 3 agents |
| (n = 10) | mean ± S.E.M.) | (%) | control | combination |
| hIgG1 + 2A3 + | 1939.17 ± 423.36 | / | / | / |
| Vehicle (10 + 1) | | | | |
| mg/kg | | | | |
| 10 mg/kg 18B10 | 1244.02 ± 196.27 | 35.85 | 0.15362 | 0.00144 |
| RMP1-14 + | 783.29 ± 180.11 | 59.61 | 0.02174 | 0.08615 |
| Oxaliplatin/5FU | | | | |
| (1 + 1/5) mg/kg | | | | |
| 18B10 + RMP1- | 401.77 ± 108.26 | 79.28 | 0.00246 | / |
| 14 + Oxaliplatin/ | | | | |
| 5FU (10 + 1 + | | | | |
| 1/5) mg/kg | | | | |

### Example 5: IHC Based Evaluation of CLDN18.2 and PD-L1 Expression in Clinical and Patient-Derived Xenograft (PDX) Samples Across Various Tumor Types

### CLDN18.2 and PD-L1 Co-Expression Status in Clinical Cancer Samples

To investigate the expression level and overlapping status of CLDN18.2 and PD-L1 in gastric cancer and cholangiocarcinoma, Immunohistochemistry (IHC) was performed on these 4% neutral buffered formalin fixed paraffin-embedded (FFPE) tumor sections using in-house developed and validated recombinant anti-CLDN18.2(14G11) and commercially available anti-PD-L1(22C3) monoclonal antibodies. After deparaffinization and rehydration, all sections were proceeded to antigen retrieval by boiling in EnVision™ FLEX Target Retrieval Solution (Dako, K8002) for 25 minutes at 97-99° C., subsequently quenched, blocked with EnVision™ FLEX Peroxidase-Blocking Reagent (Dako, K8002) and incubated with appropriately diluted 14G11(0.6 ug/mL) and 22C3(1 ug/mL) antibodies, respectively. Antibody binding was visualized with EnVision™ FLEX+, Mouse (LINKER), followed by EnVision™ FLEX/HRP and EnVision™ FLEX Substrate Working Solution (Dako, K8002). Sections were finally counterstained with Hematoxylin and mounted with permanent mounting medium.

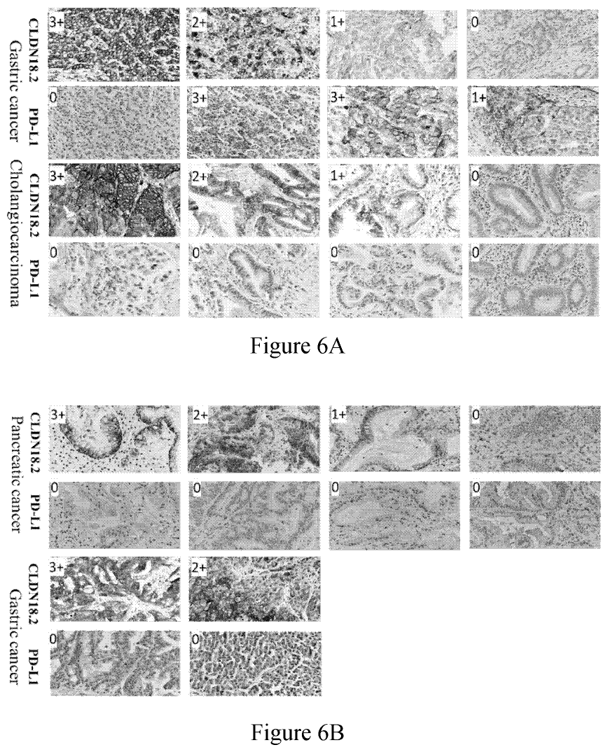

All samples were scored by the relative proportion of positive stained tumor cell relative to all visible tumor cell for CLDN18.2 or combined positive score (CPS) of total stained immune and tumor cell relative to all visible tumor cell for PD-L1 with membrane staining of different intensity (neg (0), weak (1+), moderate (2+), strong (3+)) as depicted in Table 6. Only membrane staining with ≥2+ intensity was considered as positive. Weak to strong membrane signals of CLDN18.2 and PD-L1 were generated by 14G11 and 22C3 in gastric and cholangiocarcinoma cancer tissues respectively (see FIG. 6A). The CLDN18.2 and PD-L1 expression levels and overlapping across gastric and cholangiocarcinoma cancer tissues were summarized in Table 7. The result shows that most samples have CLDN18.2-expressing in gastric and cholangiocarcinoma cancer. Furthermore, there is 93.75% of PD-L1 no or low expression rate among CLDN18.2-expressing (IHC Scoring ≥1+) tumor samples in gastric and 100% of PD-L1 no or low expression rate among CLDN18.2-expressing (IHC Scoring ≥1+) tumor samples in cholangiocarcinoma cancer.

TABLE 6
IHC result interpretation

| | | Positive | Positive |
|---|---|---|---|
| IHC | | percentage | percentage |
| Scoring | Staining intensity | (CLDN18.2) | (PD-L1) |
| 0 | Negative to faint | TC < 1% | CPS < 1% |
| 1+ | Faint to weak | 1% ≤ TC < 40% | 1% ≤ CPS < 5% |
| 2+ | Weak to moderate | 40% ≤ TC < 75% | CPS ≥ 5% |
| 3+ | Moderate to strong | TC > 75% | CPS ≥ 5% |

Note.
TC: tumor cell;
CPS: combined positive score

TABLE 7
CLDN18.2 and PD-L1 Expression Overlapping Status
in Clinical Samples across Different Cancer Type

| | | CLDN18.2 | | | | | | | | |
|---|---|---|---|---|---|---|---|---|---|---|
| Tumor | Sample | (14G11) IHC | PD-L1(22C3) IHC score (%) | | | | | | | |
| type | case | score | 0 | | 1+ | | 2+ | | 3+ | |
| Gastric | 73 | 0 | 23 | (92.00%) | 2 | (8.00%) | 0 | (0%) | 0 | (0%) |
| cancer | | 1+ | 11 | (84.62%) | 0 | (0%) | 1 | (7.69%) | 1 | (7.69%) |
| | | 2+ | 19 | (90.48%) | 1 | (4.76%) | 0 | (0%) | 1 | (4.76%) |
| | | 3+ | 12 | (85.71%) | 2 | (14.29%) | 0 | (0%) | 0 | (0%) |
| Cholangiocarcinoma | 44 | 0 | 16 | (100.00%) | 0 | (0%) | 0 | (0%) | 0 | (0%) |
| | | 1+ | 11 | (100.00%) | 0 | (0%) | 0 | (0%) | 0 | (0%) |
| | | 2+ | 8 | (100.00%) | 0 | (0%) | 0 | (0%) | 0 | (0%) |
| | | 3+ | 9 | (100.00%) | 0 | (0%) | 0 | (0%) | 0 | (0%) |

### CLDN18.2 and PD-L1 Co-Expression Status in PDX Samples

Biotinylated 14G11 (14G11 Biotin) and 22C3(22C3 Biotin) was prepared. Briefly, Biotinamidocaproate NHS ester (Sigma, B2643-10MG) was prepared in anhydrous DMF at 20 mg/mL as stock solution. 10 μl stock solution was added for each 1 mg 14G11 antibody to be labelled and mix gently for 1 hr at room temperature. Reaction products of low molecular weight were removed by desalting the product on Zeba™ Spin Desalting Columns (ThermoFisher, 89890) according to the manufacturer's instruction.

Immunohistochemistry (IHC) was performed on slides of 4% neutral buffered formalin fixed paraffin embedded PDX samples including gastric and pancreatic cancers. After deparaffinization and rehydration, all slides were proceeded to antigen retrieval by boiling in EnVision™ FLEX Target Retrieval Solution (Dako, K8002) for 25 minutes at 97-99° C., subsequently quenched, blocked with IHC Biotin Block Kit (MaiXin, BLK-0001) following instruction and incubated with 10 μg/mL in-house biotinylated monoclonal mouse anti-claudin 18.2 (14G11 Biotin) and anti-PD-L1(22C3 Biotin) antibody for 30 min at 37° C., respectively. Antibody binding was visualized with horseradish peroxidase labeled streptavidin (MaiXin, SP KIT-D1) and EnVision™ FLEX Substrate Working Solution (Dako, K8002). Sections were finally counterstained with Hematoxylin and mounted with permanent mounting medium. IHC result was scored regarding the staining intensity, pattern and positive proportion (see Table 6).

The CLDN18.2 and PD-L1 expression levels and overlapping across gastric and pancreatic PDX cancer tissues were summarized in Table 8. The result shows that most samples have CLDN18.2-expressing in gastric and pancreatic cancer. Furthermore, there is 100% of PD-L1 no or low expression among CLDN18.2-expressing (IHC Scoring ≥1+, see FIG. 6B)) tumor samples both in gastric and pancreatic cancer.

TABLE 8
CLDN18.2 and PD-L1 Expression Overlapping Status
in PDX Samples including Gastric and Pancreatic

| | Sample | CLDN18.2 (14G11) | PD-L1(22C3) IHC score | | | | |
|---|---|---|---|---|---|---|---|
| PDX type | case | IHC score | 0 | | 1+ | 2+ | 3+ |
| Pancreatic | 12 | 0 | 6 | (50.00%) | 0 (0%) | 0 (0%) | 0 (0%) |
| cancer | | 1+ | 2 | (16.67%) | 0 (0%) | 0 (0%) | 0 (0%) |
| | | 2+ | 2 | (16.67%) | 0 (0%) | 0 (0%) | 0 (0%) |
| | | 3+ | 2 | (16.67%) | 0 (0%) | 0 (0%) | 0 (0%) |
| Gastric | 4 | 0 | 0 | (0%) | 0 (0%) | 0 (0%) | 0 (0%) |
| cancer | | 1+ | 0 | (0%) | 0 (0%) | 0 (0%) | 0 (0%) |
| | | 2+ | 1 | (25%) | 0 (0%) | 0 (0%) | 0 (0%) |
| | | 3+ | 3 | (75%) | 0 (0%) | 0 (0%) | 0 (0%) |

### Example 6: Efficacy of 18B10 Combination with PD-1 Antibody on GC-02-0007 PDX Tumor Model on HSC-NSG-hIL-15 Mice

GC-02-0007 gastric tumor tissue was obtained from Beijing Cancer Hospital passage in nude mice and established PDX bank. The expression level of CLDN18.2 and PD-L1 on the GC-02-0007 PDX tumor model was measured as described in the section “CLDN18.2 and PD-L1 Co-expression Status in PDX Samples” of Example 5, and the results were shown in Table 8 “Gastric cancer”. HSC-NSG-hIL-15 mice were human HSC reconstructed on human IL-15 mice that could maintain certain human NK cell ratio purchased from JAX lab. Each mouse was subcutaneously inoculated with a small tumor tissue block approximately 3 mm in diameter which sheared from integrated tumor decollement from a tumor bearing moue. Several days after inoculation animals with tumor size at about 50 mm\hat{ }3 were selected and randomly divided into 4 groups, each group consisting of 5 mice. Then the mice were treated with 30 mg/kg isotype control, 20 mg/kg 18B10, 10 mg/kg Nivolumab, and combination of 18B10 and Nivolumab twice a week for 4 weeks by i.p. injection. Animals were sacrificed at the end of the study with CO2 inhalation. Tumor size was measured twice or triple times a week in two dimensions using a caliper (INSIZE) and the volume was expressed in mm\hat{ }3 using the formula: V=0.5 a*b\hat{ }2 where a and b are the long and short diameters of the tumor, respectively. Results were analyzed using Prism GraphPad and expressed as mean±S.E.M. Comparisons between two groups were made by T-test, and the difference is considered significant if p is *<0.05 and **<0.01.

Treatment with combination of 18B10 and Nivolumab reduces tumor volume more effectively than treatment with Nivolumab alone.

### Example 7: Efficacy of 18B10-HaLa in Combination with Anti-PD-1 and Chemo on NUGC4-hCLDN18.2 and Human PBMC Co-Inoculation Tumor Model

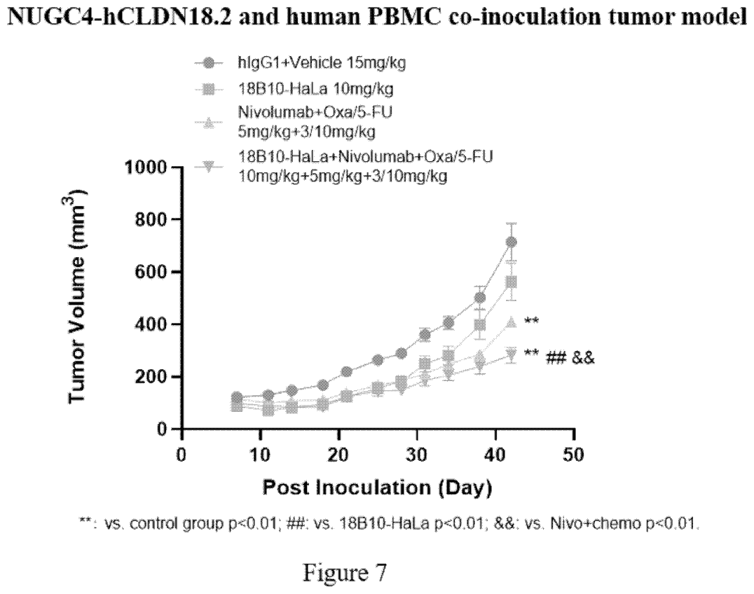

Human gastric cancer cell line NUGC4 was transfected with CLDN18.2 gene screened stable expressing CLDN18.2 was named NUGC4-hCLDN18.2. NUGC4-hCLDN18.2 cells were maintained in vitro as a monolayer culture in RPMI1640 medium (Thermo Fisher) supplemented with 10% heat inactivated fetal bovine serum (ExCell Biology), 100 U/ml penicillin, 100 ug/ml streptomycin (Hyclone) and 1 ug/mL puromycin (Gibco) at 37° C. in an atmosphere with 5% CO₂ in air. The tumor cells were routinely sub-cultured twice weekly by trypsin-EDTA treatment (Hyclone). The cells growing in an exponential growth phase were harvested and counted for tumor inoculation. Female SPF grade NOD-SCID mice were inoculated with mixed 5*10\hat{ }6 NUGC4-hCLDN18.2 cells and 5*10\hat{ }6 human PBMC with 50% matrigel. Approximately 4 hours after inoculation, mice were selected and randomized to 4 groups (n=10). Animals were treated with isotype control and vehicle, 10 mg/kg 18B10-HaLa, 5 mg/kg Nivolumab plus 3 mg/kg Oxaliplatin/10 mg/kg 5-FU and combination of 10 mg/kg 18B10-HaLa and 5 mg/kg Nivolumab plus 3 mg/kg Oxaliplatin/10 mg/kg 5-FU, twice a week for 6 weeks by i.p. injection for antibody, once weekly for 6 weeks by i.v. injection for chemo. Tumor size was measured twice or triple times a week in two dimensions using a caliper (INSIZE) and the volume was expressed in mm\hat{ }3 using the formula: V=0.5 a*b\hat{ }2 where a and b are the long and short diameters of the tumor, respectively. Results were analyzed using Prism GraphPad and expressed as mean±S.E.M. Comparisons between two groups were made by T-test, and the difference was considered significant if p was *<0.05 and **<0.01. Three-agent combination therapy had significantly enhanced antitumor activity than 18B10-HaLa alone (p<0.01) and anti-PD-1 plus chemo combination (p<0.01), as shown in Table 9 and FIG. 7.

TABLE 9
Efficacy of 18B10- HaLa in combination with anti-
PD-1 and Chemo on NUGC4-hCLDN18.2 and human PBMC
co-inoculation tumor model on Day 42

| | Tumor size | | p value vs | p value vs |
|---|---|---|---|---|
| Treatment | (mm\hat{ }3, | TGI | Isotype | 3 agents |
| (n = 9) | mean ± S.E.M.) | (%) | control | combination |
| Isotype control + | 715.54 ± 70.88 | / | / | / |
| Vehicle | | | | |
| 10 mg/kg 18B10- | 564.09 ± 71.77 | 21.17 | 0.15059 | 0.00211 |
| HaLa | | | | |
| Nivolumab + | 412.14 ± 17.96 | 42.40 | 0.00060 | 0.00215 |
| Oxaliplatin/5FU | | | | |
| (5 + 3/10) mg/kg | | | | |
| 18B10-HaLa + | 283.44 ± 31.17 | 60.39 | 0.00003 | / |
| Nivolumab + | | | | |
| Oxaliplatin/5FU | | | | |
| (10 + 5 + 3/10) | | | | |
| mg/kg | | | | |

### Example 8: Efficacy of 18B10 in Combination with Anti-PD-1 and Chemo on MFC/hCLDN18.2 Tumor Model

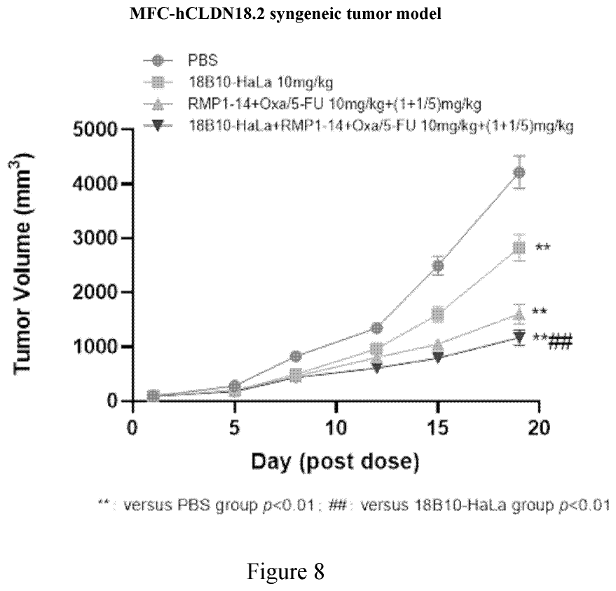

Mouse gastric cancer cell line MFC was transfected with CLDN18.2 gene screened stable expressing CLDN18.2 was named MFC/CLDN18.2. MFC/CLDN18.2 cells were maintained in vitro as a monolayer culture in DMEM medium (Thermo Fisher) supplemented with 10% heat inactivated fetal bovine serum (ExCell Biology), 100 U/ml penicillin, 100 ug/ml streptomycin (Hyclone) and 1 ug/mL puromycin (Gibco) at 37° C. in an atmosphere with 5% CO₂ in air. The tumor cells were routinely sub-cultured twice weekly by trypsin-EDTA treatment (Hyclone). The cells growing in an exponential growth phase were harvested and counted for tumor inoculation. Female SPF grade 615 mice were inoculated with 2*10\hat{ }6 MFC/CLDN18.2 cells. Approximately 5 days after inoculation and the tumor size of around 90 mm\hat{ }3, tumor bearing mice were selected and randomized to 4 groups (n=9). Animals were treated with PBS, 10 mg/kg 18B10-HaLa, 1 mg/kg RMP1-14 plus 1 mg/kg Oxaliplatin/5 mg/kg 5-FU and combination of 10 mg/kg 18B10-HaLa and 1 mg/kg RMP1-14 plus 1 mg/kg Oxaliplatin/5 mg/kg 5-FU, twice a week for 3 weeks by i.p. injection for antibody, once weekly for 3 weeks by i.v. injection for chemo. Tumor size was measured twice or triple times a week in two dimensions using a caliper (INSIZE) and the volume was expressed in mm\hat{ }3 using the formula: V=0.5 a*b\hat{ }2 where a and b are the long and short diameters of the tumor, respectively. Results were analyzed using Prism GraphPad and expressed as mean±S.E.M. Comparisons between two groups were made by T-test, and the difference was considered significant if p was *<0.05 and **<0.01. Three-agent combination therapy had enhanced antitumor activity than 18B10-HaLa alone (p<0.01) and anti-PD-1 plus chemo combination (TGI on Day 19: 72.22% versus 62.02%), as shown in Table 10 and FIG. 8.

TABLE 10
Efficacy of 18B10 combination with anti-PD-1 and
Chemo on MFC/CLDN18.2 tumor model on Day 19

| | Tumor size | | p value vs | p value vs |
|---|---|---|---|---|
| Treatment | (mm\hat{ }3, | TGI | Isotype | 3 agents |
| (n = 9) | mean ± S.E.M.) | (%) | control | combination |
| PBS | 4215.07 ± 302.18 | / | / | / |
| 10 mg/kg 18B10- | 2821.81 ± 245.98 | 33.05 | 0.0025252 | 0.00003 |
| HaLa | | | | |
| RMP1-14 + | 1600.84 ± 182.01 | 62.02 | 0.0000015 | 0.08479 |
| Oxaliplatin/5FU | | | | |
| (1 + 1/5) mg/kg | | | | |
| 18B10-HaLa + | 1170.83 ± 147.11 | 72.22 | 0.0000001 | / |
| RMP1-14 + | | | | |
| Oxaliplatin/5FU | | | | |
| (10 + 1 + 1/5) | | | | |
| mg/kg | | | | |

TABLE 11
Amino acid sequences mentioned in the present disclosure

| SED ID NO. | Sequences | Region |
|---|---|---|
| 1 | GYNMN | 18B10_HCDR1 |
| 2 | NIDPYYGGTSYNQKFKG | 18B10_HCDR2 |
| 3 | MYHGNAFDY | 18B10_HCDR3 |
| 4 | KSSQSLLNSGNLKNYLT | 18B10_LCDR1 |
| 5 | WASTRKS | 18B10_LCDR2 |
| 6 | QNDYSYPLT | 18B10_LCDR3 |
| 7 | EFQLQQSGPELEKPGASVRISCKTSGYSFTGYN | 18B10-VH |
| | MNWVKQSNGESLEWIGNIDPYYGGTSYNQKFK | |
| | GKATLTVDKSSSTAYMQLKSLTSEDSAVYYCAR | |
| | MYHGNAFDYWGQGTTLTVSS | |
| 8 | DIVMTQSPSSLTVTAGEKVTMSCKSSQSLLNSGN | 18B10-VL |
| | LKNYLTWYQQKPGQPPKLLIYWASTRKSGVPDR | |
| | FTGSGSGTDFTLTLSSVQAEDLAVYYCQNDYSY | |
| | PLTFGAGTKLELK | |
| 9 | ASTKGPSVFPLAPSSKSTSGGTAALGCLVKDYFP | Human IgG1 |
| | EPVTVSWNSGALTSGVHTFPAVLQSSGLYSLSSV | heavy chain |
| | VTVPSSSLGTQTYICNVNHKPSNTKVDKKVEPK | constant region |
| | SCDKTHTCPPCPAPELLGGPSVFLFPPKPKDTLMI | |
| | SRTPEVTCVVVDVSHEDPEVKFNWYVDGVEVH | |
| | NAKTKPREEQYNSTYRVVSVLTVLHQDWLNGK | |
| | EYKCKVSNKALPAPIEKTISKAKGQPREPQVYTL | |
| | PPSRDELTKNQVSLTCLVKGFYPSDIAVEWESNG | |
| | QPENNYKTTPPVLDSDGSFFLYSKLTVDKSRWQ | |
| | QGNVFSCSVMHEALHNHYTQKSLSLSPGK | |
| 10 | RTVAAPSVFIFPPSDEQLKSGTASVVCLLNNFYP | Human Kappa |
| | REAKVQWKVDNALQSGNSQESVTEQDSKDSTY | light chain constant |
| | SLSSTLTLSKADYEKHKVYACEVTHQGLSSPVT | region |
| | KSFNRGEC | |
| 11 | ASTKGPSVFPLAPSSKSTSGGTAALGCLVKDYFP | Sequences of |
| | EPVTVSWNSGALTSGVHTFPAVLQSSGLYSLSSV | engineered Fc |
| | VTVPSSSLGTQTYICNVNHKPSNTKVDKKVEPK | L235V/F243L/R292P/ |
| | SCDKTHTCPPCPAPELVGGPSVFLLPPKPKDTLM | Y300L/P396L |
| | ISRTPEVTCVVVDVSHEDPEVKFNWYVDGVEV | |
| | HNAKTKPPEEQYNSTLRVVSVLTVLHQDWLNG | |
| | KEYKCKVSNKALPAPIEKTISKAKGQPREPQVYT | |
| | LPPSRDELTKNQVSLTCLVKGFYPSDIAVEWESN | |
| | GQPENNYKTTPLVLDSDGSFFLYSKLTVDKSRW | |
| | QQGNVFSCSVMHEALHNHYTQKSLSLSPGK | |
| 12 | QVQLVQSGAEVKKPGASVKVSCKASGYTFTGY | Hu18B10-Ha |
| | NMNWVRQAPGQGLEWMGNIDPYYGGTSYNQK | |
| | FKGRVTMTIDKSTSTVYMELSSLRSEDTAVYYC | |
| | ARMYHGNAFDYWGQGTTVTVSS | |
| 13 | QVQLVQSGAEVKKPGASVKVSCKASGYSFTGY | Hu18B10-Hb |
| | NMNWVRQAPGQGLEWMGNIDPYYGGTSYNQK | |
| | FKGRVTLTIDKSTSTVYMELSSLRSEDTAVYYCA | |
| | RMYHGNAFDYWGQGTTVTVSS | |
| 14 | QVQLVQSGAEVKKPGASVKVSCKASGYSFTGY | Hu18B10-Hc |
| | NMNWVKQAPGQGLEWIGNIDPYYGGTSYNQK | |
| | FKGRVTLTIDKSTSTVYMELSSLRSEDTAVYYCA | |
| | RMYHGNAFDYWGQGTTVTVSS | |
| 15 | DIVMTQSPDSLAVSLGERATINCKSSQSLLNSGN | Hu18B10_La |
| | LKNYLTWYQQKPGQPPKLLIYWASTRKSGVPDR | |
| | FSGSGSGTDFTLTISSLQAEDVAVYYCQNDYSYP | |
| | LTFGGGTKVEIK | |
| 16 | DIVMTQSPDSLAVSLGERATMNCKSSQSLLNSG | Hu18B10_Lb |
| | NLKNYLTWYQQKPGQPPKLLIYWASTRKSGVP | |
| | DRFTGSGSGTDFTLTISSLQAEDVAVYYCQNDYS | |
| | YPLTFGGGTKVEIK | |
| 17 | QVQLVQSGAEVKKPGASVKVSCKASGYVFTDY | AM4B6_VH |
| | YMNWVRQAPGQSLEWMGDINPNNAETLYNHK | |
| | FKGRVTVTVDKSISTAYMELSRLRSDDTAVYYC | |
| | VKWGDGPFAYWGQGTLVTVSS | |
| 18 | DIQMTQSPSSLSASVGDRVTITCKASQNVGAAV | AM4B6_VL |
| | AWYQQKPGKAPKLLIYSVSDRYTGVPSRFSGSG | |
| | SGTDFTLTISSLQPEDIATYYCQQYSNYPTFGQG | |
| | TKLEIK | |
| 19 | QVQLVQSGAEVKKPGASVKVSCKASGYVFTDY | AM4B6_HC_1 |
| | YMNWVRQAPGQSLEWMGDINPNNAETLYNHK | |
| | FKGRVTVTVDKSISTAYMELSRLRSDDTAVYYC | |
| | VKWGDGPFAYWGQGTLVTVSSASTKGPSVFPL | |
| | APSSKSTSGGTAALGCLVKDYFPEPVTVSWNSG | |
| | ALTSGVHTFPAVLQSSGLYSLSSVVTVPSSSLGT | |
| | QTYICNVNHKPSNTKVDKKVEPKSCDKTHTCPP | |
| | CPAPEFEGGPSVFLFPPKPKDTLMISRTPEVTCV | |
| | VVDVSHEDPEVKFNWYVDGVEVHNAKTKPRE | |
| | EQYNSTYRVVSVLTVLHQDWLNGKEYKCKVS | |
| | NKALPASIEKTISKAKGQPREPQVYTLPPSRDEL | |
| | TKNQVSLTCLVKGFYPSDIAVEWESNGQPENNY | |
| | KTTPPVLDSDGSFFLYSKLTVDKSRWQQGNVFS | |
| | CSVMHEALHNHYTQKSLSLSPGA | |
| 20 | QVQLVQSGAEVKKPGASVKVSCKASGYVFTDY | AM4B6_HC_2 |
| | YMNWVRQAPGQSLEWMGDINPNNAETLYNHK | |
| | FKGRVTVTVDKSISTAYMELSRLRSDDTAVYYC | |
| | VKWGDGPFAYWGQGTLVTVSSASTKGPSVFPL | |
| | APSSKSTSGGTAALGCLVKDYFPEPVTVSWNSG | |
| | ALTSGVHTFPAVLQSSGLYSLSSVVTVPSSSLGT | |
| | QTYICNVNHKPSNTKVDKKVEPKSCDKTHTCPP | |
| | CPAPELLGGPSVFLFPPKPKDTLMISRTPEVTCV | |
| | VVDVSHEDPEVKFNWYVDGVEVHNAKTKPRE | |
| | EQYNSTYRVVSVLTVLHQDWLNGKEYKCKVS | |
| | NKALPAPIEKTISKAKGQPREPQVYTLPPSRDEL | |
| | TKNQVSLTCLVKGFYPSDIAVEWESNGQPENNY | |
| | KTTPPVLDSDGSFFLYSKLTVDKSRWQQGNVFS | |
| | CSVMHEALHNHYTQKSLSLSPGK | |
| 21 | DIQMTQSPSSLSASVGDRVTITCKASQNVGAAV | AM4B6_LC |
| | AWYQQKPGKAPKLLIYSVSDRYTGVPSRFSGSG | |
| | SGTDFTLTISSLQPEDIATYYCQQYSNYPTFGQG | |
| | TKLEIKRTVAAPSVFIFPPSDEQLKSGTASVVCLL | |
| | NNFYPREAKVQWKVDNALQSGNSQESVTEQDS | |
| | KDSTYSLSSTLTLSKADYEKHKVYACEVTHQGL | |
| | SSPVTKSFNRGEC | |
| 22 | DYYMN | AM4B6_HCDR1 |
| 23 | DINPNNAETLYNHKFKG | AM4B6_HCDR2 |
| 24 | WGDGPFAY | AM4B6_HCDR3 |
| 25 | KASQNVGAAVA | AM4B6_LCDR1 |
| 26 | SVSDRYT | AM4B6_LCDR2 |
| 27 | QQYSNYPT | AM4B6_LCDR3 |
| 28 | QVQLVQSGAEVKKPGASVKLSCKASGYIFTTY | 23F11_VH |
| | WMHWVKQRPGQGLEWIGMIQPNSGGTKYNEK | |
| | FKKKATLTVDKSISTAYMELSRLTSDDTAVYYCA | |
| | RGAGTVDYFDYWGQGSTLTVSS | |
| 29 | DIVLTQSPASLAVSVGQRATITCRASESVDIYGNS | 23F11_VL |
| | FMHWYQQKPGQPPKLLIYRASNLESGIPARFSGS | |
| | GSRTDFTLTINPVEAQDTATYYCQQSTEDPYTFG | |
| | GGTKLEIK | |
| 30 | QVQLVQSGAEVKKPGASVKLSCKASGYIFTTY | 23F11_HC |
| | WMHWVKQRPGQGLEWIGMIQPNSGGTKYNEK | |
| | FKKKATLTVDKSISTAYMELSRLTSDDTAVYYCA | |
| | RGAGTVDYFDYWGQGSTLTVSSASTKGPSVFPL | |
| | APSSKSTSGGTAALGCLVKDYFPEPVTVSWNSG | |
| | ALTSGVHTFPAVLQSSGLYSLSSVVTVPSSSLGTQ | |
| | TYICNVNHKPSNTKVDKKVEPKSCDKTHTCPPC | |
| | PAPELLGGPSVFLFPPKPKDTLMISRTPEVTCVV | |
| | VDVSHEDPEVKFNWYVDGVEVHNAKTKPREE | |
| | QYASTYRVVSVLTVLHQDWLNGKEYKCKVSNK | |
| | ALPAPIEKTISKAKGQPREPQVYTLPPSRDELTKN | |
| | QVSLTCLVKGFYPSDIAVEWESNGQPENNYKTT | |
| | PPVLDSDGSFFLYSKLTVDKSRWQQGNVFSCSV | |
| | MHEALHNHYTQKSLSLSPGK | |
| 31 | DIVLTQSPASLAVSVGQRATITCRASESVDIYGNS | 23F11_LC |
| | FMHWYQQKPGQPPKLLIYRASNLESGIPARFSGS | |
| | GSRTDFTLTINPVEAQDTATYYCQQSTEDPYTFG | |
| | GGTKLEIKRTVAAPSVFIFPPSDEQLKSGTASVVC | |
| | LLNNFYPREAKVQWKVDNALQSGNSQESVTEQ | |
| | DSKDSTYSLSSTLTLSKADYEKHKVYACEVTHQ | |
| | GLSSPVTKSFNRGEC | |
| 32 | TYWMH | 23F11_HCDR1 |
| 33 | MIQPNSGGTKYNEKFKK | 23F11_HCDR2 |
| 34 | GAGTVDYFDY | 23F11_HCDR3 |
| 35 | RASESVDIYGNSFMH | 23F11_LCDR1 |
| 36 | RASNLES | 23F11_LCDR2 |
| 37 | QQSTEDPYT | 23F11_LCDR3 |
| 38 | MAVTACQGLGFVVSLIGIAGIIAATCMDQWSTQ | Amino acid |
| | DLYNNPVTAVFNYQGLWRSCVRESSGFTECRGY | sequence of human |
| | FTLLGLPAMLQAVRALMIVGIVLGAIGLLVSIFAL | CLDN18.2 |
| | KCIRIGSMEDSAKANMTLTSGIMFIVSGLCAIAG | |
| | VSVFANMLVTNFWMSTANMYTGMGGMVQTV | |
| | QTRYTFGAALFVGWVAGGLTLIGGVMMCIACR | |
| | GLAPEETNYKAVSYHASGHSVAYKPGGFKASTG | |
| | FGSNTKNKKIYDGGARTEDEVQSYPSKHDYV | |
| 39 | QVQLVQSGAEVKKPGASVKVSCKASGYTFTGY | 18B10 Heavy |
| | NMNWVRQAPGQGLEWMGNIDPYYGGTSYNQK | chain |
| | FKGRVTMTIDKSTSTVYMELSSLRSEDTAVYYC | |
| | ARMYHGNAFDYWGQGTTVTVSSASTKGPSVFP | |
| | LAPSSKSTSGGTAALGCLVKDYFPEPVTVSWNS | |
| | GALTSGVHTFPAVLQSSGLYSLSSVVTVPSSSLGT | |
| | QTYICNVNHKPSNTKVDKKVEPKSCDKTHTCPP | |
| | CPAPELLGGPSVFLFPPKPKDTLMISRTPEVTCVV | |
| | VDVSHEDPEVKFNWYVDGVEVHNAKTKPREE | |
| | QYNSTYRVVSVLTVLHQDWLNGKEYKCKVSNK | |
| | ALPAPIEKTISKAKGQPREPQVYTLPPSRDELTKN | |
| | QVSLTCLVKGFYPSDIAVEWESNGQPENNYKTT | |
| | PPVLDSDGSFFLYSKLTVDKSRWQQGNVFSCSV | |
| | MHEALHNHYTQKSLSLSPGK | |
| 40 | DIVMTQSPDSLAVSLGERATINCKSSQSLLNSGN | 18B10 Light chain |
| | LKNYLTWYQQKPGQPPKLLIYWASTRKSGVPDR | |
| | FSGSGSGTDFTLTISSLQAEDVAVYYCQNDYSYP | |
| | LTFGGGTKVEIK | |
| | RTVAAPSVFIFPPSDEQLKSGTASVVCLLNNFYP | |
| | REAKVQWKVDNALQSGNSQESVTEQDSKDSTY | |
| | SLSSTLTLSKADYEKHKVYACEVTHQGLSSPVT | |
| | KSFNRGEC | |
| 41 | QVQLQQSGPELVRPGASVKISCKASGYRFTRNY | Heavy chain of |
| | FHWVKQRPGQGLEWIGWIYPGGFDIEYSEKFKG | recombinant anti- |
| | KATLTTDTSSSTAYMLLTSLTSEDSAVYYCAINY | CLDN18.2(14G11) |
| | GSTFGYWGQGTLVTVSVAKTTPPSVYPLAPGSA | |
| | AQTNSMVTLGCLVKGYFPEPVTVTWNSGSLSSG | |
| | VHTFPAVLQSDLYTLSSSVTVPSSTWPSQTVTCN | |
| | VAHPASSTKVDKKIVPRDCGCKPCICTVPEVSSV | |
| | FIFPPKPKDVLTITLTPKVTCVVVDISKDDPEVQF | |
| | SWFVDDVEVHTAQTKPREEQINSTFRSVSELPIM | |
| | HQDWLNGKEFKCRVNSAAFPAPIEKTISKTKGR | |
| | PKAPQVYTIPPPKEQMAKDKVSLTCMITNFFPED | |
| | ITVEWQWNGQPAENYKNTQPIMDTDGSYFVYS | |
| | KLNVQKSNWEAGNTFTCSVLHEGLHNHHTEKS | |
| | LSHSPGK | |
| 42 | DVLMTQTPLSLPVSLGDQASISCRSSQNIVHSNG | Light chain of |
| | NTYLEWYLQRPGQSPKLLIYKVSNRFSGVPDRF | recombinant anti- |
| | SGSGSGTDFTLKINRVEAEDLGVYYCFQGSHVP | CLDN18.2(14G11) |
| | FTFGSGTKLEIKRADAAPTVSIFPPSSEQLTSGGA | |
| | SVVCFLNNFYPKDINVKWKIDGSERQNGVLNS | |
| | WTDQDSKDSTYSMSSTLTLTKDEYERHNSYTCE | |
| | ATHKTSTSPIVKSFNRNEC | |
| 43 | QVQLQQSGPELVRPGASVKISCKASGYRFTRNY | Heavy chain |
| | FHWVKQRPGQGLEWIGWIYPGGFDIEYSEKFKG | variable region of |
| | KATLTTDTSSSTAYMLLTSLTSEDSAVYYCAINY | recombinant anti- |
| | GSTFGYWGQGTLVTVSV | CLDN18.2(14G11) |
| 44 | DVLMTQTPLSLPVSLGDQASISCRSSQNIVHSNG | Light chain |
| | NTYLEWYLQRPGQSPKLLIYKVSNRFSGVPDRF | variable region of |
| | SGSGSGTDFTLKINRVEAEDLGVYYCFQGSHVP | recombinant anti- |
| | FTFGSGTKLEIK | CLDN18.2(14G11) |
| 45 | DSWIH | Atezolizumab_HCDR1 |
| 46 | WISPYGGSTYYADSVKG | Atezolizumab_HCDR2 |
| 47 | RHWPGGFDY | Atezolizumab_HCDR3 |
| 48 | RASQDVSTAVA | Atezolizumab_LCDR1 |
| 49 | SASFLYS | Atezolizumab_LCDR2 |
| 50 | QQYLYHPAT | Atezolizumab_LCDR3 |
| 51 | EVQLVESGGGLVQPGGSLRLSCAASGFTFSDSWI | Atezolizumab_VH |
| | HWVRQAPGKGLEWVAWISPYGGSTYYADSVKG | |
| | RFTISADTSKNTAYLQMNSLRAEDTAVYYCARR | |
| | HWPGGFDYWGQGTLVTVSS | |
| 52 | DIQMTQSPSSLSASVGDRVTITCRASQDVSTAVA | Atezolizumab_VL |
| | WYQQKPGKAPKLLIYSASFLYSGVPSRFSGSGSG | |
| | TDFTLTISSLQPEDFATYYCQQYLYHPATFGQGT | |
| | KVEIK | |
| 53 | EVQLVESGGGLVQPGGSLRLSCAASGFTFSDSWI | Atezolizumab_HC |
| | HWVRQAPGKGLEWVAWISPYGGSTYYADSVKG | |
| | RFTISADTSKNTAYLQMNSLRAEDTAVYYCARR | |
| | HWPGGFDYWGQGTLVTVSSASTKGPSVFPLAPS | |
| | SKSTSGGTAALGCLVKDYFPEPVTVSWNSGALT | |
| | SGVHTFPAVLQSSGLYSLSSVVTVPSSSLGTQTYI | |
| | CNVNHKPSNTKVDKKVEPKSCDKTHTCPPCPAP | |
| | ELLGGPSVFLFPPKPKDTLMISRTPEVTCVVVDV | |
| | SHEDPEVKFNWYVDGVEVHNAKTKPREEQYAS | |
| | TYRVVSVLTVLHQDWLNGKEYKCKVSNKALPA | |
| | PIEKTISKAKGQPREPQVYTLPPSREEMTKNQVS | |
| | LTCLVKGFYPSDIAVEWESNGQPENNYKTTPPVL | |
| | DSDGSFFLYSKLTVDKSRWQQGNVFSCSVMHE | |
| | ALHNHYTQKSLSLSPGK | |
| 54 | DIQMTQSPSSLSASVGDRVTITCRASQDVSTAVA | Atezolizumab_LC |
| | WYQQKPGKAPKLLIYSASFLYSGVPSRFSGSGSG | |
| | TDFTLTISSLQPEDFATYYCQQYLYHPATFGQGT | |
| | KVEIKRTVAAPSVFIFPPSDEQLKSGTASVVCLLN | |
| | NFYPREAKVQWKVDNALQSGNSQESVTEQDSK | |
| | DSTYSLSSTLTLSKADYEKHKVYACEVTHQGLS | |
| | SPVTKSFNRGEC | |
| 55 | NSGMH | Nivolumab_HCDR1 |
| 56 | VIWYDGSKRYYADSVKG | Nivolumab_HCDR2 |
| 57 | NDDY | Nivolumab_HCDR3 |
| 58 | RASQSVSSYLA | Nivolumab_LCDR1 |
| 59 | DASNRAT | Nivolumab_LCDR2 |
| 60 | QQSSNWPRT | Nivolumab_LCDR3 |
| 61 | QVQLVESGGGVVQPGRSLRLDCKASGITFSNSG | Nivolumab_VH |
| | MHWVRQAPGKGLEWVAVIWYDGSKRYYADSV | |
| | KGRFTISRDNSKNTLFLQMNSLRAEDTAVYYCA | |
| | TNDDYWGQGTLVTVSS | |
| 62 | EIVLTQSPATLSLSPGERATLSCRASQSVSSYLAW | Nivolumab_VL |
| | YQQKPGQAPRLLIYDASNRATGIPARFSGSGSGT | |
| | DFTLTISSLEPEDFAVYYCQQSSNWPRTFGQGTK | |
| | VEIK | |
| 63 | QVQLVESGGGVVQPGRSLRLDCKASGITFSNSG | Nivolumab_HC |
| | MHWVRQAPGKGLEWVAVIWYDGSKRYYADSV | |
| | KGRFTISRDNSKNTLFLQMNSLRAEDTAVYYCA | |
| | TNDDYWGQGTLVTVSSASTKGPSVFPLAPCSRS | |
| | TSESTAALGCLVKDYFPEPVTVSWNSGALTSGV | |
| | HTFPAVLQSSGLYSLSSVVTVPSSSLGTKTYTCN | |
| | VDHKPSNTKVDKRVESKYGPPCPPCPAPEFLGG | |
| | PSVFLFPPKPKDTLMISRTPEVTCVVVDVSQEDP | |
| | EVQFNWYVDGVEVHNAKTKPREEQFNSTYRVV | |
| | SVLTVLHQDWLNGKEYKCKVSNKGLPSSIEKTI | |
| | SKAKGQPREPQVYTLPPSQEEMTKNQVSLTCLV | |
| | KGFYPSDIAVEWESNGQPENNYKTTPPVLDSDG | |
| | SFFLYSRLTVDKSRWQEGNVFSCSVMHEALHNH | |
| | YTQKSLSLSLGK | |
| 64 | EIVLTQSPATLSLSPGERATLSCRASQSVSSYLAW | Nivolumab_LC |
| | YQQKPGQAPRLLIYDASNRATGIPARFSGSGSGT | |
| | DFTLTISSLEPEDFAVYYCQQSSNWPRTFGQGTK | |
| | VEIKRTVAAPSVFIFPPSDEQLKSGTASVVCLLNN | |
| | FYPREAKVQWKVDNALQSGNSQESVTEQDSKD | |
| | STYSLSSTLTLSKADYEKHKVYACEVTHQGLSSP | |
| | VTKSFNRGEC | |

1. A method of treating a CLDN18.2-associated disease or condition in a subject in need thereof, comprising:
administering to the subject a therapeutically effective amount of a CLDN18.2 antagonist in combination with a therapeutically effective amount of PD-1/PD-L1 axis inhibitor,
wherein the subject is determined to have CLDN18.2 expression in a diseased tissue.

2. The method of claim 1, further comprising administering to the subject a therapeutically effective amount of a chemotherapeutic agent.

3. The method of claim 1, wherein the CLDN18.2 expression in the diseased tissue is higher than or comparable to expression in healthy or noncancerous stomach cells or stomach tissue.

4. The method of claim 1, wherein the CLDN18.2 expression in the diseased tissue is lower than expression in healthy or noncancerous stomach cells or stomach tissue but higher than expression in a healthy or noncancerous tissue or organ other than stomach.

5. The method of claim 1, wherein the CLDN18.2 expression in the diseased tissue is comparable to expression in a healthy or noncancerous tissue or organ other than stomach, and is detectable by an anti-CLDN18.2 diagnostic antibody.

6. (canceled)

7. The method of claim 1, wherein the subject is determined to have PD-L1 expression in the diseased tissue.

8. The method of claim 1, wherein the subject is determined to have low or no PD-L1 expression in the diseased tissue.

9. The method of claim 8, wherein the PD-L1 expression in the diseased tissue is lower than a reference level.

10. The method of claim 8, wherein no more than 20% of the cells of the diseased tissue are positive for PD-L1 expression.

11. The method of claim 10, wherein the cells of a diseased tissue comprise disease cells and immune cells in the diseased tissue.

12. The method of claim 8, wherein the PD-L1 expression in the diseased tissue is comparable to that in a healthy or noncancerous tissue, or the PD-L1 expression in the diseased tissue is low or non-detectable by an anti-PD-L1 diagnostic antibody.

13. (canceled)

14. A method of sensitizing a disease or condition to a treatment with PD-1/PD-L1 axis inhibitor in a subject in need thereof, wherein the subject is determined to have low or no expression of PD-L1 in a diseased tissue, the method comprising:
a) determining presence or expression level of CLDN18.2 in the diseased tissue obtained from the subject; and
b) when the presence of CLDN18.2 is determined in step a) or when the expression level of CLDN18.2 reaches a threshold level in step a), administering to the subject a therapeutically effective amount of a CLDN18.2 antagonist, optionally in combination with a therapeutically effective amount of a PD-1/PD-L1 axis inhibitor,
thereby sensitizing the disease or condition to a treatment with PD-1/PD-L1 axis inhibitor.

15. The method of claim 14, wherein the step b) further comprises administering to the subject a chemotherapeutic agent.

16-21. (canceled)

22. A method for increasing responsiveness of a tumor to a treatment with PD-1/PD-L1 axis inhibitor in a subject, wherein the subject is determined to have a tumor resistant or refractory to the treatment with a PD-1/PD-L1 axis inhibitor, comprising:
a) determining presence or expression level of CLDN18.2 in a tumor sample obtained from the subject; and
b) when the presence of CLDN18.2 is determined in step a) or when the expression level of CLDN18.2 reaches a threshold level in step a), administering to the subject a therapeutically effective amount of a CLDN18.2 antagonist, and optionally in combination with a therapeutically effective amount of a PD-1/PD-L1 axis inhibitor,
thereby increasing responsiveness of the tumor to the treatment with PD-1/PD-L1 axis inhibitor in the subject.

23. The method of claim 22, wherein the step b) further comprises administering to the subject a chemotherapeutic agent.

24-31. (canceled)

32. The method of claim 1, wherein the CLDN18.2 antagonist comprises an anti-CLDN18.2 antibody, such as a monoclonal anti-CLDN18.2 antibody, a bi-specific antibody targeting CLDN18.2 and a second antigen (e.g., CD3, 4-1BB, TGFβ, SIRPα, and IL15), or immune cells expressing chimeric antigen receptors (CARs) or genetically modified TCRs comprising an anti-CLDN18.2 antigen binding domain.

33. The method of claim 32, wherein the anti-CLDN18.2 antibody comprises heavy chain HCDR1, HCDR2 and HCDR3 and/or light chain LCDR1, LCDR2 and LCDR3 sequences, wherein:
the HCDR1 sequence comprises GYNMN (SEQ ID NO: 1), or a homologue sequence of at least 80% sequence identity thereof;
the HCDR2 sequence comprises NIDPYYGGTSYNQKFKG (SEQ ID NO: 2), or a homologue sequence of at least 80% sequence identity thereof;
the HCDR3 sequence comprises MYHGNAFDY (SEQ ID NO: 3), or a homologue sequence of at least 80% sequence identity thereof;
the LCDR1 sequence comprises KSSQSLLNSGNLKNYLT (SEQ ID NO: 4) or a homologue sequence of at least 80% sequence identity thereof;
the LCDR2 sequence comprises WASTRKS (SEQ ID NO: 5) or a homologue sequence of at least 80% sequence identity thereof;
the LCDR3 sequence comprises QNDYSYPLT (SEQ ID NO: 6) or a homologue sequence of at least 80% sequence identity thereof.

34. The method of claim 33, wherein the anti-CLDN18.2 antibody comprises a heavy chain variable region and a light chain variable region, wherein
the heavy chain variable region comprises an amino acid sequence of SEQ ID NO: 7, and the light chain variable region comprises an amino acid sequence of SEQ ID NO: 8.

35-38. (canceled)

39. The method of claim 32, wherein the constant region comprises one or more amino acid residue substitutions relative to SEQ ID NO: 9, selected from the group consisting of: L235V, F243L, R292P, Y300L, P396L, or any combination thereof.

40. The method of claim 39, wherein the constant region comprises the sequence of SEQ ID NO: 11, optionally the constant region further comprises the sequence of SEQ ID NO: 10.

41-42. (canceled)

43. The method of claim 39, wherein the anti-CLDN18.2 antibody comprises a heavy chain variable region and a light chain variable region, wherein
the heavy chain variable region comprises an amino acid sequence selected from the group consisting of SEQ ID NO: 12, SEQ ID NO: 13 and SEQ ID NO: 14, and
the light chain variable region comprises an amino acid sequence selected from the group consisting of SEQ ID NO: 15 and SEQ ID NO: 16.

44. The method of claim 32, wherein the anti-CLDN18.2 antibody comprises a heavy chain and a light chain, wherein
the heavy chain comprises an amino acid sequence of SEQ ID NO: 39, and
the light chain comprises an amino acid sequence of SEQ ID NO: 40.

45. The method of claim 32, wherein the anti-CLDN18.2 antibody is capable of inducing the expression of PD-L1 in the diseased tissue of the subject.

46-47. (canceled)

48. The method of claim 1, wherein the PD-1/PD-L1 axis inhibitor comprises PD-1 inhibitor selected from the group consisting of antibody, small molecule, and combination thereof, or the PD-1/PD-L1 axis inhibitor comprises PD-L1 inhibitor selected from the group consisting of antibody, small molecule, and combination thereof.

49. The method of claim 48, wherein
(i) the PD-1 inhibitor comprises an anti-PD-1 antibody selected from the group consisting of: Nivolumab (OPDIVO; BMS-936558), Dostarlimab (TSR-042), Pembrolizumab (KEYTRUDA; MK-3475), MEDI0680 (AMP-514), MEDI4736, BI 754091, Pidilizumab (CT-011), Cemiplimab (LIBTAYO, REGN2810), Spartalizumab (PDR001), Cetrelimab (JNJ 63723283), Toripalimab (JS001), PF-06801591, Tislelizumab (BGB-A317), AMP-224 (GSK-2661380), ABBV-181, Lambrolizumab, Camrelizumab (SHR-1210), Sintilimab (Tyvyt, IBI308), Penpulimab(AK105), Zimberelimab, Retifanlimab, Serplulimab, Balstilimab, Geptanolimab, Prolgolimab, Ezabenlimab, Sasanlimab, Pimivalimab, Budigalimab, Nofazinlimab, Sindelizumab, MGA404, Sym021, BAT1306, HX008; or
(ii) the PD-L1 inhibitor comprises an anti-PD-L1 antibody selected from the group consisting of: Atezolizumab (TECENTRIQ; R05541267; MPDL3280A; RG7446), BMS-936559, Avelumab (bavencio), lodapolimab (LY3300054), Durvalumab (MEDI4736), CX-072 (Proclaim-CX-072), FAZ053, Envafolimab(KN035), MDX-1105, STI-1040, CS1001, Adebrelimab (SHR-1316), SHR-1701, TOB2450, Bintrafusp, LP002, STI-3031, Cosibelimab, Pacmilimab, NM01, LDP, AMP-224, Garivulimab(BGB-A333), A167,SCD-135, Opucolimab, GR1405; optionally the PD-L1 inhibitor is Atezolizumab (TECENTRIQ; R05541267; MPDL3280A; RG7446); or
the PD-L1 inhibitor comprises a bispecific antibody targeting both PD-L1 and another checkpoint molecule selected from the group consisting of PD-1, PD-L1, PD-L2, CLTA-4, SIRP, TIM-3, LAG3, A2AR, CD160, 2B4, TGFβ, VISTA, BTLA, TIGIT, LAIR1, OX40, CD2, CD27, CD28, CD30, CD40, CD122, ICAM-1, IDO, NKG2C, SLAMF7, SIGLEC7, NKp80, CD160, B7-H3, LFA-1, 1COS, 4-1BB, GITR, BAFFR, HVEM, CD7, LIGHT, IL-2, IL-15, CD3, CD16 or CD83.

50-55. (canceled)

56. The method of claim 1, wherein the PD-L1 inhibitor comprises an anti-PD-L1 antibody comprising heavy chain HCDR1, HCDR2 and HCDR3 and/or light chain LCDR1, LCDR2 and LCDR3 sequences, wherein:
(a) the HCDR1 sequence comprises DYYMN (SEQ ID NO: 22), or a homologue sequence of at least 80% sequence identity thereof;
the HCDR2 sequence comprises DINPNNAETLYNHKFKG (SEQ ID NO: 23), or a homologue sequence of at least 80% sequence identity thereof;
the HCDR3 sequence comprises WGDGPFAY (SEQ ID NO: 24), or a homologue sequence of at least 80% sequence identity thereof;
the LCDR1 sequence comprises KASQNVGAAVA (SEQ ID NO: 25) or a homologue sequence of at least 80% sequence identity thereof;
the LCDR2 sequence comprises SVSDRYT (SEQ ID NO: 26) or a homologue sequence of at least 80% sequence identity thereof;
the LCDR3 sequence comprises QQYSNYPT (SEQ ID NO: 27) or a homologue sequence of at least 80% sequence identity thereof; or
(b) the HCDR1 sequence comprises TYWMH (SEQ ID NO: 32), or a homologue sequence of at least 80% sequence identity thereof;
the HCDR2 sequence comprises MIQPNSGGTKYNEKFKK (SEQ ID NO: 33), or a homologue sequence of at least 80% sequence identity thereof;
the HCDR3 sequence comprises GAGTVDYFDY (SEQ ID NO: 34), or a homologue sequence of at least 80% sequence identity thereof;
the LCDR1 sequence comprises RASESVDIYGNSFMH (SEQ ID NO: 35) or a homologue sequence of at least 80% sequence identity thereof;
the LCDR2 sequence comprises RASNLES (SEQ ID NO: 36) or a homologue sequence of at least 80% sequence identity thereof;
the LCDR3 sequence comprises QQSTEDPYT (SEQ ID NO: 37) or a homologue sequence of at least 80% sequence identity thereof.

57-59. (canceled)

60. The method of claim 56, wherein the PD-L1 inhibitor comprises an anti-PD-L1 antibody comprising a heavy chain variable region and a light chain variable region, wherein
(a) the heavy chain variable region comprises an amino acid sequence of SEQ ID NO: 17, and
the light chain variable region comprises an amino acid sequence of SEQ ID NO: 18; or
(b) the heavy chain variable region comprises an amino acid sequence of SEQ ID NO: 28, and
the light chain variable region comprises an amino acid sequence of SEQ ID NO: 29.

61. The method of claim 60, wherein the PD-L1 inhibitor comprises an anti-PD-L1 antibody comprising a heavy chain and a light chain, wherein
(a) the heavy chain comprises an amino acid sequence of SEQ ID NO: 19 or SEQ ID NO: 20, and
the light chain comprises an amino acid sequence of SEQ ID NO: 21; or
(b) the heavy chain comprises an amino acid sequence of SEQ ID NO: 30, and
the light chain comprises an amino acid sequence of SEQ ID NO: 31.

62-64. (canceled)

65. The method of claim 1, wherein the disease or condition is cancer, or the diseased tissue comprises a cancer cell.

66. (canceled)

67. The method of claim 65, wherein the cancer is selected from the group consisting of gastric cancer, lung cancer, bronchial cancer, bone cancer, liver and bile duct cancer, pancreatic cancer, breast cancer, liver cancer, ovarian cancer, testicle cancer, kidney cancer, bladder cancer, head and neck cancer, spine cancer, brain cancer, cervix cancer, uterine cancer, endometrial cancer, colon cancer, colorectal cancer, rectal cancer, anal cancer, esophageal cancer, gastrointestinal cancer, skin cancer, prostate cancer, pituitary cancer, stomach cancer, vagina cancer, thyroid cancer, glioblastoma, astrocytoma, melanoma, myelodysplastic syndrome, sarcoma, teratoma, and adenocarcinoma.

68. (canceled)

69. The method of claim 1, wherein the disease or condition is resistant or refractory to a treatment with a PD-1/PD-L1 axis inhibitor, optionally the resistance is de novo or acquired.

70-71. (canceled)

72. The method of claim 69, wherein the disease or condition is resistant or refractory to a combinatory therapy with a PD-1/PD-L1 axis inhibitor and a chemotherapeutic agent.

73. The method of claim 72, wherein the chemotherapeutic agent is selected from the group consisting of: antimetabolites such as methotrexate and 5-fluorouracil (5-FU), Oxaliplatin, alkylating agents (e.g., thiotepa and cyclophosphamide (Cytoxan™), alkyl sulfonates (e.g., busulfan, improsulfan and piposulfan), aziridines (e.g., benzodopa, carboquone, meturedopa, and uredopa), emylerumines and memylamelamines (e.g., altretamine, triemylenemelamine, triethylenephosphoramide, triethylenethiophosphoramide, and trimemylolomelamine), acetogenins, a camptothecin (e.g., synthetic analogue topotecan), bryostatin, callystatin, CC-1065 (including its adozelesin, carzelesin and bizelesin synthetic analogues), cryptophycins (articularly cryptophycin and cryptophycin), dolastatin, duocarmycin (including the synthetic analogues, KW-2 189 and CBI-TMI), eleutherobin, pancratistatin, a sarcodictyin, spongistatin, cisplatin, nitrogen mustards (e.g., chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard), nitrosoureas (e.g., carmustine, chlorozotocin, foremustine, lomustine, nimustine, ranimustine, folic acid analogues (e.g., demopterin, methotrexate, pteropterin, trimetrexate, purine analogs (e.g., fludarabine, 6-mercaptopurine, thiamiprine, thioguanine, pyrimidine analogues (e.g., ancitabine, azacitidine, 6-azauridine, carmofur, cytarabine, dideoxyuridine, doxifluridine, enocitabine, floxuridine), androgens (e.g., calusterone, dromostanolone propionate, epitiostanol, mepitiostane, testolactone), anti-adrenals (e.g., aminoglutethimide, mitotane, trilostane), folic acid replinisher (e.g., frolinic acid), aceglatone, aldophosphamide glycoside, aminolevulinic acid, eniluracil, amsacrine, hestrabucil, bi santrene, edatrexate, defofamine, demecolcine, diaziquone, elformthine, elliptinium acetate, an epothilone, etoglucid, gallium nitrate, hydroxyurea, lentinan, lonidamine, maytansinoids (e.g., maytansine and ansamitocins), mitoguazone, mitoxantrone, mopidamol, nitracrine, pentostatin, phenamet, pirarubicin, losoxantrone, podophyllinic acid, 2-ethylhydrazide, procarbazine, P SK™, razoxane, rhizoxin, sizofiran, spirogermanium, tenuazonic acid, triaziquone, 2,2,2″-tricUorotriemylamine, trichothecenes, urethane, vindesine, dacarbazine, mannomustine, mitobronitol, mitolactol.

74. A kit useful in treating a disease or condition in a subject in need thereof, comprising a first container that comprises a CLDN18.2 antagonist and a second container that comprises a PD-1/PD-L1 axis inhibitor, and optionally instructions for use of the kit,
wherein the disease or condition is characterized in having: a) CLDN18.2 expression in a diseased tissue, and/or b) low or no expression of PD-L1 in the diseased tissue.

75. A kit, comprising a CLDN18.2 antagonist and a package insert comprising instructions for using the CLDN18.2 antagonist in combination with a PD-1/PD-L1 axis inhibitor to treat a disease or condition in a subject in need thereof,
wherein the disease or condition is characterized in having: a) CLDN18.2 expression in a diseased tissue, and/or b) low or no expression of PD-L1 in the diseased tissue.

76-78. (canceled)
